(19) United States
(12) Patent Application Publication
(10) Pub. No.: US 2004/0110825 A1
(43) Pub. Date: Jun. 10, 2004

(54) Method for treating sepsis

(76) Inventors: Andrew Loh, Carmel, IN (US); William Louis Macias, Indianapolis, IN (US); Simona Skerjanec, Pittstown, NJ (US)

Correspondence Address: ELI LILLY AND COMPANY, PATENT DIVISION, P.O. BOX 6288, INDIANAPOLIS, IN, 46206-6288, US

(21) Appl. No.: 10/332,178

(22) PCT Filed: Jun. 29, 2001
(86) PCT No.: PCT/US01/16509

(51) Int. Cl. A61K 31/405

(52) U.S. Cl. 514/419

(57) ABSTRACT

A novel method of treating and/or preventing sepsis.

### FIELD OF THE INVENTION

This invention relates to a method for the treatment and/or prevention of sepsis or septic shock.

### BACKGROUND OF THE INVENTION

The structure and physical properties of human non-pancreatic secretory phospholipase A₂ (hereinafter called, “sPLA₂”) has been thoroughly described in two articles, namely, “Cloning and Recombinant Expression of Phospholipase A₂ Present in Rheumatoid Arthritic Synovial Fluid” by Seilhamer, Jeffrey J.; Pruzanski, Waldemar; Vadas Peter; Plant, Shelley; Miller, Judy A.; Kloss, Jean; and Johnson, Lorin K.; The Journal of Biological Chemistry, Vol. 264, No. 10, Issue of April 5, pp. 5335-5338, 1989; and “Structure and Properties of a Human Non-pancreatic Phospholipase A₂” by Kramer, Ruth M.; Hession, Catherine; Johansen, Berit; Hayes, Gretchen; McGray, Paula; Chow, E. Pingchang; Tizard, Richard; and Pepinsky, R. Blake; The Journal of Biological Chemistry, Vol. 264, No. 10, Issue of April 5, pp. 5768-5775, 1989; the disclosures of which are incorporated herein by reference.

It is believed that sPLA₂ is a rate limiting enzyme in the arachidonic acid cascade which hydrolyzes membrane phospholipids. Membrane phospholipids have in turn been implicated in inflammatory diseases and diseases caused by systemic response to injury and/or inflammation (see Uhl, et al., J. Am. Coll. Surg. 331, 180, 3, 323-331, 1993)). Thus, it is important to develop compounds and methods of treatment which inhibit sPLA₂ mediated release of fatty acids (e.g., arachidonic acid) and which are highly bioavailable in mammals, especially humans. Such compounds and methods are of value in the general treatment of conditions induced and/or maintained by overproduction of sPLA₂; such as septic shock, adult respiratory distress syndrome, pancreatitis, trauma, bronchial asthma, allergic rhinitis, rheumatoid arthritis, etc.

Sepsis and its symptoms afflict a number variously estimated at between about 500,000 and 2 million people worldwide a year, with a mortality rate of about 30-50%. It is associated with an overwhelming systemic response to viral, fungal or bacterial infection, and is characterized often by sudden chills, pneumonia, trauma, and infections during surgery and infections to burn victims. It is frequent for example, among patients with cancer, and AIDS, and more so when such patients are confined in hospital or clinical settings. There is as yet no approved treatment for sepsis. Approximately 100,000 people die from sepsis each year. The human and economic costs of affliction with sepsis and symptoms thereof, are enormous.

U.S. Pat. No. 5,654,326 incorporated by reference describes certain indole type sPLA₂ inhibitors and related ester prodrugs useful for the treatment of sepsis. In particular, this patent exemplifies the sodium salt, and methyl ester of ((3-(2-amino-1,2-dioxoethyl)-2-methyl-1-(phenylmethyl)-1H-indol-4-yl)oxy)acetic acid. U.S. Patent provisional application number S No. 60/063,646 filed Oct. 27, 1997 describes a highly bioavailable indole type sPLA₂ inhibitor for the treatment of sepsis. U.S. patent application Ser. No. 09/063,066 filed Apr. 21, 1998 (titled, “Substituted Carbazoles and 1,2,3,4-Tetrahydrocarbazoles”), discloses carbazole type compounds useful for the treatment of sepsis, the entire disclosure of which is incorporated herein by reference. U.S. patent application Ser. No. 09/260,490 filed Mar. 3, 1998 discloses certain lyophilized formulations of sPLA₂ inhibitor compounds, the entire disclosure of which is incorporated herein by reference.

It is desirable to develop specialized methods for treating and/or preventing sepsis using highly bioavailable sPLA₂ inhibitors, particularly those suitable for oral or intravenous administration in combination with novel methods of intervention and/or prevention by medical professionals or competent caregivers.

### SUMMARY OF THE INVENTION

The present invention relates to a method for the use of sPLA₂ inhibitor compounds for the treatment of sepsis involving administration of a sPLA₂ inhibitor compound within a specific time interval.

The present invention is method of treating or preventing sepsis in a patient having sepsis or susceptible to sepsis respectively, by administering a sPLA₂ inhibitor compound prior to the onset of sepsis or up to 24 hours after first organ failure and continuing as medically or clinically necessary.

The present invention relates to a method for the use of a sPLA₂ inhibitor compound for the treatment of sepsis wherein the active ingredient is administered within 0 to 24 hours after first organ failure and continued for about 1 to 7 days or until a medically determined stopping point.

The present invention relates to a method for the use of a sPLA₂ inhibitor compound for the treatment of sepsis wherein the active ingredient is administered within 0 to 18 hours after first organ failure and continued for about 1 to 7 days or until a medically determined stopping point.

The present invention also provides a method for preventing or treating sepsis comprising the steps of:

a. selecting patient susceptible to sepsis;

b. monitoring sPLA₂ activity levels in patient;

administering effective amount of a compound of formula I or II if sPLA₂ activity levels are high or on the rise.

The present invention relates to a method for the use of a sPLA₂ inhibitor compound for the treatment of sepsis wherein the active ingredient is administered within 0 to 12 hours after first organ failure and continued for about 1 to 7 days or until a medically determined stopping point.

The present invention relates to a method for the use of a sPLA₂ inhibitor compound for the treatment of sepsis wherein the active ingredient is administered within 0 to 6 hours after first organ failure and continued for about 1 to 7 days or until a medically determined stopping point.

The present invention relates to a method for the use of a sPLA₂ inhibitor compound for the treatment of sepsis wherein the active ingredient is administered within 0 to 2 hours after first organ failure and continued for about 1 to 7 days or until a medically determined stopping point.

The present invention also relates to a method of preventing sepsis in a mammal including a human, susceptible to sepsis wherein administration of a sPLA₂ inhibitor compound is initiated prior to the onset of elevated sPLA₂ levels.

The present invention provides sPLA₂ inhibitor compounds to a patient prior to invasive procedure(s), which may lead to sepsis or are capable of causing the injurious conditions that lead to sepsis.

The present invention is also a method of treating or preventing sepsis by administering a therapeutically effective amount of a sPLA₂ compound in combination with other effective therapy for sepsis within 24 hours after first organ failure and continuing administration for about 1 to 7 days or as medically necessary.

The present invention is a method of treating or preventing sepsis comprising initiating administration of a pharmaceutical formulation comprising a sPLA₂ inhibitor to a patient susceptible to sepsis prior to organ failure or within about 24 hours after organ failure to a patient afflicted with sepsis.

The present invention is a method for the use of a compound of formula I for the treatment or prevention of sepsis by the method of the invention, wherein the compound of formula I is

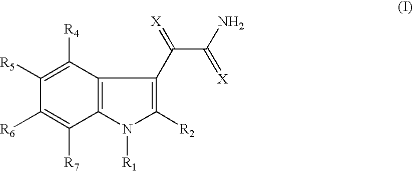

wherein R₁ thru R₇ and X are defined infra.

The present invention is also method for the use of the sPLA₂ inhibitor compound of formula II for the treatment or prevention of sepsis by the method of the invention, wherein the compound of formula II is

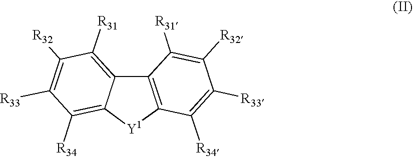

wherein R₃₁ thru R₃₄, R₃₁′ thru R₃₄′ and yl are defined infra.

The present invention is also a method for the treatment or prevention of sepsis comprising initiating administration of a pharmaceutical formulation comprising a compound of formula I or II in combination with a carrier or diluent according to the method of the invention.

The present invention is also a method for the treatment or prevention of sepsis in a patient afflicted with sepsis or susceptible to sepsis comprising initiating administration of a pharmaceutical formulation comprising a compound of formula I or II in combination with other effective therapy or co-agent for sepsis within 24 hours after first organ failure or prior to a rise in sPLA₂ levels.

The present invention relates to a method of preventing sepsis in a mammal including a human, said method comprising initiating administration to a patient susceptible to sepsis a pharmaceutically effective amount of a sPLA₂ inhibitor compound prior to occurrence of injury causing conditions.

The present invention relates to a method of treating sepsis wherein treatment of a patient with a pharmaceutically effective amount of a sPLA₂ inhibitor compound of formula I or II is initiated within a time interval from first organ failure or onset of rise in sPLA₂ activity levels.

The present invention relates to a method of treating sepsis wherein treatment of a patient with a pharmaceutically effective amount of a sPLA₂ inhibitor compound of formula I or II or a pharmaceutically acceptable salt, solvate or prodrug derivative thereof, is initiated within a time interval from first organ failure or onset of elevated sPLA₂ levels.

The present invention relates to the use of a sPLA₂ inhibitor compound of formula I or II or a pharmaceutically acceptable salt, solvate or prodrug derivative thereof, for the manufacture of a medicament for the treatment of sepsis wherein administration of the pharmaceutically effective amount of a sPLA₂ inhibitor compound of formula I or II or a pharmaceutically acceptable salt, solvate or prodrug derivative thereof, is initiated within a time interval from first organ failure or onset of elevated sPLA₂ levels.

The present invention is the use of a compound of formula (I) or (II) in the manufacture of a medicament for the treatment or prevention of sepsis in a patient afflicted with sepsis or susceptible to sepsis comprising initiating administration of a pharmaceutical formulation comprising a compound of formula I or II within 24 hours after first organ failure or prior to a rise in sPLA₂ levels.

The present invention is also use of a compound of formula (I) or (II) in the manufacture of a medicament for the treatment or prevention of sepsis in a patient afflicted with sepsis or susceptible to sepsis comprising initiating administration of a pharmaceutical formulation comprising a compound of formula I or II in combination with other effective therapy or co-agent for sepsis within 24 hours after first organ failure or prior to a rise in sPLA₂ levels.

### THE DRAWINGS

Three figures (FIG. 1, FIG. 2, and FIG. 3) are presented.

FIG. 1 represents the relationship between 28 day mortality by treatment (Y-axis) and the sPLA₂ activity quartile or range (from less than 82 to greater than 511 ng/mL) (X-axis). Bars 1, 4, 7, and 10 represent the mortality rate when placebo was administered at the applicable sPLA₂ activity quartile. Bars 2, 5, 8, and 11 represent mortality rates for the administration of “low dose” of sPLA₂ inhibitor compound at the applicable level of sPLA₂ activity (quartile). Bars 3, 6, 9, and 12 represent treatment of patients with a “high dose” of a compound of formula (I).

FIG. 2 mirrors the experiments for which results are presented in FIG. 1, except that the 28 day mortality by treatment (Y-axis) observed in FIG. 2 is plotted against IL-6 activity quartile (activity level of between less than 107 and greater than 1506 ng/mL))(X-axis). Again, placebo administration at the different IL-6 quartiles are represented by bars 1, 4, 7, and 10. Low dose administration at the different IL-6 quartiles are represented by bars 2, 5, 8, and 11. While the results of high dose administration at the different IL-6 activity quartiles are represented by bars 3, 6, 9 and 12.

FIG. 3 represents a plot of 28-day mortality by treatment (Y-axis) against time after first organ failure at which a sPLA₂ inhibitor compound of formula I was administered (X-axis). FIG. 3 shows that at less than 12 hours after first organ failure, administration, preferably by infusion, of a sPLA₂ inhibitor compound of formula 1, preferably compound (Vb), was associated with 19.2% mortality at the “low dose” (bar 2) compared with administration of placebo having mortality of 42.9% (bar 1). Administration of a “high dose” of sPLA₂ inhibitor compound of formula (I), at the same time frame (i.e., less than 12 hours after first organ failure) was associated with a mortality of 5.4% (bar 3). The apparent reduction in mortality associated with both low dose and high dose administered at 0-12 hours after first organ failure are statistically significant and unexpected. FIG. 3, further shows that between 12 and 24 hours after first organ failure, administration of placebo was associated with a mortality of 35.1% (bar 4), compared with a “low dose” administration of a sPLA₂ compound having a mortality of 38.2% (bar 5), and also compared with administration of a “high dose” sPLA₂ compound having a mortality of 31.5% (bar 6). These data points show that administration of a sPLA₂ inhibitor compound, preferably a compound of formula 1, more preferably, compound (Vb), at 0-24 hours after first organ failure, and preferably at 0-12 hours after first organ failure, results in a significant reduction in mortality associated with sepsis when compared to placebo. The efficacy of a sPLA₂ inhibitor administered at 0-24 hours, preferably 0-12 hours, after first organ failure is particularly significant when a “high dose” sPLA₂ inhibitor compound is administered.

The data were analyzed using the Cochran-Mantel-Haenszel test adjusted for the trial primary stratification factors at a two-sided 5% significance level. The overall treatment effect as well as for pair-wise comparisons were analyzed.

### DETAILED DESCRIPTION OF THE INVENTION DEFINITIONS

As used herein the phrase “susceptible to sepsis” means a patient who independently exhibits symptom(s) associated with sepsis or in characterized by behaviors or conditions which predispose to the contraction of infections (i.e., viral, fungal or bacterial), that are capable of leading to sepsis. Examples of such behaviors and/or condition include but are not limited to acute or chronic alcoholism, AIDS, respiratory diseases, gastrointestinal disease, burns, trauma and especially when accompanied by admission to a hospital, clinical or care-giving setting(s) for related or unrelated conditions. The determination of susceptibility to sepsis is made by a competent care giver, i.e., a doctor in view of prevailing medical knowledge of symptoms and/or extent or severity of risk factors exhibited by patient as described for example in Harrison's Principles of Internal Medicine supra. Thus, not all patients are within the definition of susceptibility to sepsis, only those determined as described above.

An used herein a combination therapy of an sPLA₂ inhibitor compound and other effective medication, drug or treatment procedure according to the method of the invention, denotes the use of an sPLA₂ compound and other effective therapy in a single unit dose, separate doses given simultaneously or sequentially but within the treatment regimen of the invention, i.e., initiation of administration of sPLA₂ inhibitor compound(s) prior to or within 24 hours after first organ failure.

As used herein the terms “LY315920”, “LY315920 sodium” are synonymous with the compound of formula (Vb) infra and is also chemically known as ((2-Methyl-1-(phenylethyl)-1H-indol-4-yl)oxy)acetic acid sodium salt or other chemically equivalent name.

As used herein the prefix ‘CT” stands for clinical trial material and identifies a compound, drug, or placebo as indicated when accompanied by a number.

As used herein “sepsis” encompasses all stages of the disease or condition as characterized by standard medical reference texts and/or known to one of skill in the art. For example sepsis includes severe sepsis, septic shock, etc.

The terms “sPLA₂ inhibitor”, “sPLA₂ compound” and “sPLA₂ inhibitor compound” as used herein are synonymous.

The term “therapeutically effective amount” is a quantity of sPLA₂ inhibitor sufficient to ameliorate the symptoms secondary to sepsis in an animal.

The term “therapeutically effective interval” is a period of time beginning when one of either the sPLA₂ inhibitor or the co-agent or combination therapy or drug is administered or practiced on the patient in need thereof, and ending at the limit of the therapeutic effectiveness of either or both.

The terms “parenteral” or “parenteral administration” mean administration by a route such as subcutaneous, intramuscular, intraorbital, intracapsular, intraspinal, intrasternal, transdermal, transmucosal, transbuccal, transrectal, transvaginal, transnasal or intravenous.

The term “animal” means any member of the animal kingdom including mammals, reptiles, fishes and fowls.

As used herein, the terms “lyophilized compositions”, “pharmaceutical compositions” and “pharmaceutical preparations” refer to all preparations described in “General rules for preparation” in the Japanese Pharmacopoeia, preferably those that are solutions and injection preparations, more preferably solutions for injection and lyophilized preparations for injection.

As used herein the terms “low dose” and “low-LY” are synonymous and represent a treatment group administered a sPLA₂ inhibitor compound to achieve a target sPLA₂ plasma concentration of 200 ng/mL.

As used herein the terms “high dose” and “high-LY” are synonymous and represent a treatment group administered a sPLA₂ inhibitor compound to achieve a target sPLA₂ plasma concentration of 800 ng/mL.

The term, “lyophilized composition(s)” refers to the solid freeze-dried composition of matter prepared by the process of this invention and comprising as essential ingredients: (1) compound(s) useful for the practice of the invention i.e. sodium [[3-(2-amino-1,2-dioxoethyl)-2-ethyl-1-(phenylmethyl)-1H-indol-4-yl]oxy]acetate; (2) a Solubilizer.

The term “active compound” or “active ingredient(s) mean one or more sPLA₂ inhibitors and/or other co-agent(s) used in combination with sPAL₂ compound(s) used in the method of the invention.; (2) a solubilizer and (3) a Stabilizer.

The term, “Active Ingredient” (also called compounds useful for the practice of the invention i.e sPLA₂ inhibitor compounds including the compound, sodium [[3-(2-amino-1,2-dioxoethyl)-2-ethyl-1-(phenylmethyl)-1H-indol-4-yl]oxy]acetate, a compound represented by the formula:

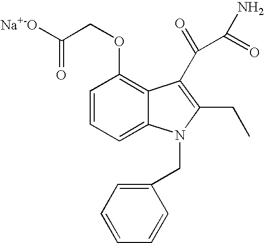

The term, “collapse temperature,” describes the glass transition temperature for amorphous solids or eutectic temperature for crystalline solids. Collapse temperature is that temperature above which the product is not completely frozen. Freeze dry microscoscropy enables measurement of the temperature at which frozen solutions begin to lose their rigid structure during a sublimation process. For sodium [[3-(2-amino-1,2-dioxoethyl)-2-ethyl-1-(phenylmethyl)-1H-indol-4-yl]oxy]acetate frozen solutions, the collapse temperature before annealing has been measured at about −33° C. while the collapse temperature after annealing is about −13° C.

The term, “Solubilizer” refers to a chelating agent. An “effective amount of Solubilizer” is a quantity of Solubilizer that permits the Active Ingredient to form stable aqueous solutions suitable for medical use.

The term, “Stabilizer” refers to a solid sugar or sugar-alcohol. An “effective amount of Stabilizer” is a quantity of Stabilizer that permits the lyophilized composition to be readily dissolved to form aqueous solutions suitable for medical use.

The term, “the equivalent acid of Active Ingredient” or “the equivalent acid of Compound (I)” means [[3-(2-Amino-1,2-dioxoethyl)-2-ethyl-1-(phenylmethyl)-1H-indol-4-yl]oxy]acetic acid. In this specification, weight of Active Ingredient is shown by the actual weight of sPLA₂ inhibitor i.e. sodium [[3-(2-amino-1,2-dioxoethyl)-2-ethyl-1-(phenylmethyl)-1H-indol-4-yl]oxy]acetate or combination therapy. In showing proportion (weight %) of Active ingredient, however, it is calculated based on the equivalent acid of Active Ingredient. Thus weights of Active Ingredient must be multiplied by the factor 100/105.8 to calculate the equivalent weight of the equivalent acid when for example active ingredient is sodium [[3-(2-amino-1,2-dioxoethyl)-2-ethyl-1-(phenylmethyl)-1H-indol-4-yl]oxy]acetate.

The term “in combination with” denotes the co-administration of a sPLA₂ inhibitor and a co-agent therapy or procedure. The term further means simultaneous co-administration either as a single formulation or as separate formulations or sequential administration of a sPLA₂ inhibitor and co-agent or other therapeutically effective procedure for the treatment or prevention of sepsis.

The term “co-agent” is a therapeutically effective medication or procedure administered in combination with a sPLA₂ inhibitor either as a single dose unit or as separate dose units, simultaneously or sequentially within a therapeutic interval.

The phrase “injury causing condition(s)” as used herein means conditions which are known to predispose one to sepsis or cause sepsis, i.e., risk factors for sepsis. These conditions have been described in the literature and are known to one of skill in the art. Descriptions of risk factors for sepsis can be found in general reference texts and literature including Harrison's Principles of Internal Medicine, thirteenth ed., 1994, pages 511-515, by McGraw-Hill, Inc., ISBN 0-07-032370-4), and in Sorensen, et al. Platelet Activating Factor and phospholipase A₂ in patients with septic shock and trauma, Intensive Care Medicine (1994) 20, 555-561.

As used herein the term “sPLA₂ activity levels” and “sPLA₂ levels” are synonymous and indicate the level of systemic, serum, or plasma sPLA₂ levels as determined by tests known to one of skill in the art and wherein a rise in these (SPLA₂) levels above about 300 units/mL (or other generally known or set normal levels depending on method of analysis) have been determined to signify a systemic response to injury correlating to different levels of sepsis depending on the severity. See Sorensen, et al., Intensive Care Medicine, 20, 555-561, (1994), for the proposition that normal plasma PLA₂ activity for healthy individuals is less than 300 units/mL.

As used herein the terms “high sPLA₂ levels,” “elevated sPLA₂ levels,” “rise in sPLA₂ levels” are synonymous and mean either single point result of sPLA₂ activity level analysis above a predetermined or generally agreed normal or average level or range, or a multi-point result of sPLA₂ activity level analysis which shows an increase in level from an initial data point.

I. sPLA₂ Inhibitors Useful in the Method of the Invention:

Secretary phopholipase A₂ (sPLA₂) inhibitors in general are useful in the practice of the method of this invention. Exemplary of classes of suitable sPLA₂ inhibitors useful in the method of the invention for treatment and/or prevention of sepsis or septic shock includes members selected from the group comprising: 1H-indole-3-glyoxylamide, 1H-indole-3-hydrazide, 1H-indole-3-acetamide, 1H-indole-1-glyoxylamide, 1H-indole-1-hydrazide, 1H-indole-1-acetamide, indolizine-1-acetamide, indolizine-1-acetic acid hydrazide, indolizine-1-glyoxylamide, indene-1-acetamide, indene-1-acetic acid hydrazide, indene-1-glyoxylamide, carbazole, tetrahydrocarbazole, pyrazole, phenyl glyoxamide, pyrrole, naphthyl glyoxamide, naphthyl acetamide, phenyl acetamide, pyrrolo[1,2-a]pyrazine, 9H-carbazole, 9-benzylcarbazole and mixtures thereof.

The 1H-indole-3-glyoxylamide Inhibitors

The 1H-indole-3-glyoxylamide sPLA₂ inhibitors and method of making them are described in U.S. Pat. No. 5,654,326, the disclosure of which is incorporated herein by reference. These 1H-indole-3-glyoxylamide compounds are also described in European Patent Application No. 95302166.4, Publication No.0675110 (publ., 4 Oct. 1995).

Definitions for 1H-indole-3-glyoxylamide Compounds:

The words, “acid linker” refers to a divalent linking group of the 1H-indole-3-glyoxylamide compounds is symbolized as, -(Lₐ)-, which has the function of joining the 4 or 5 position of the indole nucleus to an acidic group in the general relationship:

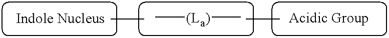

The words, “acid linker length”, refer to the number of atoms (excluding hydrogen) in the shortest chain of the linking group -(Lₐ)- that connects the 4 or 5 position of the indole nucleus with the acidic group.

The method of the invention includes a method for treatment of sepsis in an animal including a human, within 24 hours after first organ failure due to sepsis, or prevention of sepsis in an animal including a human, susceptible to sepsis prior to organ failure or rise in sPLA₂ levels.

This invention includes administering to said animal a therapeutically effective amount of a 1H-indole-3-glyoxylamide represented by the formula (I), or a pharmaceutically acceptable salt or aliphatic ester prodrug derivative thereof within the time frame (interval) of the method of the invention;

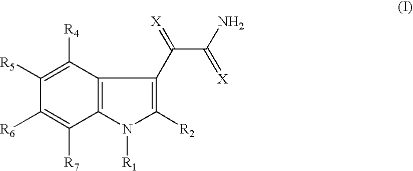

where;

X is oxygen,

R¹ is selected from the group consisting of —C₇-C₂₀ alkyl,

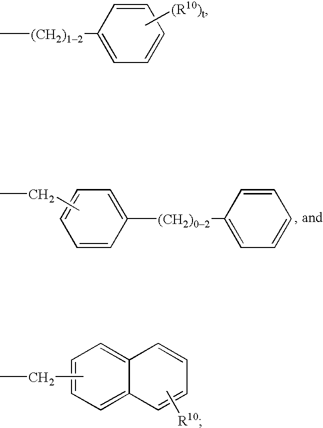

where

R¹⁰ is selected from the group consisting of halo, (C₁-C₁₀)alkyl, (C₁-C₁₀)alkoxy, —S—(C₁-C₁₀ alkyl) and halo(C₁-C₁₀)alkyl, and t is an integer from 0 to 5 both inclusive;

R₂ is selected from the group consisting of hydrogen, halo, cyclopropyl, methyl, ethyl, and propyl;

R₄ and R₅ are independently selected from the group consisting of hydrogen, a non-interfering substituent and the group, -(Lₐ)-(acidic group); where,

at least one of R₄ and R₅ is the group, -(Lₐ)- (acidic group) and wherein the (acidic group) is selected from the group consisting of —CO₂H, —SO₃H, or —P(O)(OH)₂; where,

-(Lₐ)- is an acid linker with the proviso that;

the acid linker group, -(Lₐ)-, for R₄ is selected from the group consisting of

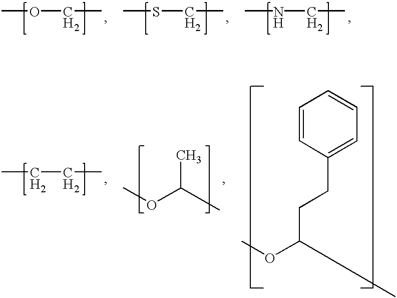

and

where R¹⁰³ is a non-interfering substituent, and where,

the acid linker, -(Lₐ), for R⁵ is selected from the group consisting of

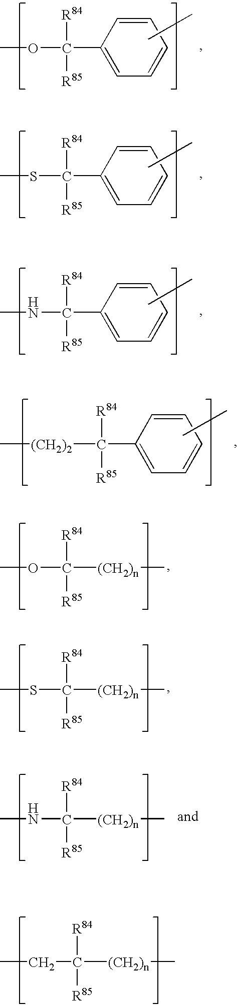

where R⁸⁴ and R⁸⁵ are each independently selected from hydrogen, C₁-C₁₀ alkyl, aryl, C₁-C₁₀ alkaryl, C₁-C₁₀ arylkyl, carboxy, carbalkoxy, and halo and,

R₆ and R₇ are each independently selected from hydrogen and non-interfering substituents, where non-interfering substituents are selected from the group consisting of C₁-C₆ alkyl, C₂-C₆ alkenyl, C₂-C₆ alkynyl, C₇-C₁₂ arylenalkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between 1 and 8, and R is hydrogen, alkyl, aryl or arylakyl.

The method of the invention also includes prevention of sepsis in an animal, including human, susceptible to sepsis by initiating administration of an sPLA₂ inhibitor compound prior to a rise in sPLA₂ levels, or treatment of an animal afflicted with sepsis by initiating administration of a sPLA₂ inhibitor compound within 24 hours after first organ failure. This method includes administering to said animal a therapeutically effective amount of a 9H-carbazole compound represented by the formula (II), or a pharmaceutically acceptable salt or aliphatic ester prodrug derivative thereof;

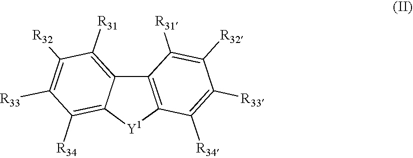

where Y¹ is selected from the group consisting of O, NH, NR¹ and S;

R¹ is selected from the group consisting of —(C₇-C₂₀)alkyl,

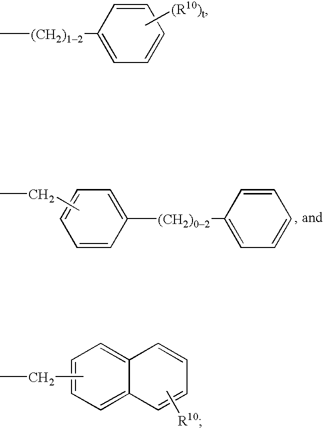

where

R¹⁰ is selected from the group consisting of halo, (C₁-C₁₀)alkyl, (C₁-C₁)alkoxy, —S—(C₁-C₁₀ alkyl) and halo(C₁-C₁₀)alkyl, and t is an integer from 0 to 5 both inclusive;

where R₃₁, R₃₂, R₃₃, R_{31′}, R_{32′}, R_{33′}, R₃₄ and R_{34′} are independently selected from the group consisting of hydrogen, CONR¹⁰¹R¹⁰², alkyl, alkylaryl, aryl, alkylheteroaryl, haloalkyl, alkylCONR¹⁰¹R¹⁰², a non-interfering substituent, and the group, -(Lₐ)-(acidic group);

where -(Lₐ)- is an acid linker selected from the group consisting of

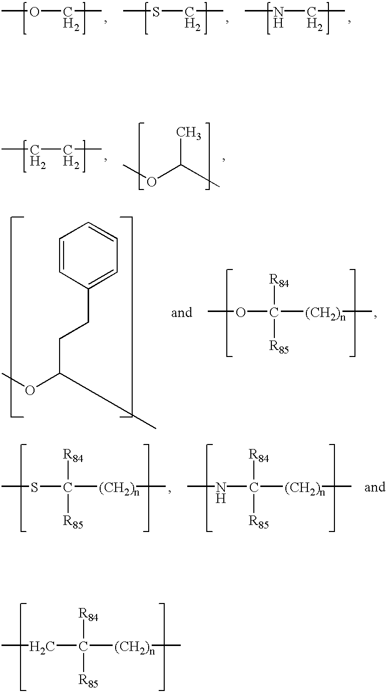

where R⁸⁴ and R⁸⁵ are each independently selected from the group consisting of hydrogen, C₁-C₁₀ alkyl, aryl, C₁-C₁₀ alkaryl, C₁-C₁₀ aralkyl, carboxy, carbalkoxy, and halo; and n is 1 or 2 and,

where the (acidic group) is selected from the group consisting of —CO₂H, —SO₃H, —CO₂NR¹⁰¹R¹⁰² and —P(O)(OH)₂ and,

where R¹⁰¹ and R¹⁰² are independently selected from the group consisting of hydrogen, alkyl, aryl, heteroaryl and haloalkyl and,

where non-interfering substituents are selected from the group consisting of C₁-C₆ alkyl, C₂-C₆ alkenyl, C₂-C₆ alkynyl, C₇-C₁₂ arylalkyl, C₇-C₁₂ alkylaryl, C₃-C₉ cycloalkyl, C₃-C₈ cycloalkyl, phenyl, tolulyl, xylyl, biphenyl, C₁-C₆ alkoxy, C₂-C₆ alkyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, C(O)O(C₁-C₆ alkyl), —(CH₂)ₙ—O—(C₁-C₆ alkyl), benzyloxy, phenoxy, phenylthio, —(CONHSO₂(R)), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between about 1 and 8 and,

R is selected from the group consisting of hydrogen and alkyl and,

where at least one of R₃₁, R₃₂, R₃₃ or R₃₄ is the group -(Lₐ)-(acidic group)

The method of the invention also includes treatment of an animal including human, afflicted with sepsis within 24 hours after first organ failure, or prevention of sepsis in an animal including human, susceptible to sepsis by administering a sPLA₂ inhibitor compound prior to organ failure or rise in sPLA₂ levels. This method includes administering to said animal as described above, a therapeutically effective amount of a 1H-indole-3-glyoxylamidecompound or a 9H-carbazole or a pharmaceutically acceptable salt, solvate, or a prodrug derivative thereof selected from the group consisting of compounds (A) through (AL):

(A) [[3-(2-amino-1,2-dioxoethyl)-2-methyl-l(phenylmethyl)-1H indol-4-yl]oxy]acetic acid,

(B) dl-2-[[3-(2-amino-1,2-dioxoethyl)-2-methyl-1-(phenylmethyl)-1H indol-4-yl]oxy]propanoic acid,

(C) [[3-(2-amino-1,2-dioxoethyl)-1-([1,1′-biphenyl]-2-ylmethyl)-2 methyl-1H-indol-4-yl]oxy]acetic acid,

(D) [[3-(2-amino-1,2-dioxoethyl)-1-([1,1′-biphenyl]-3-ylmethyl)-2-methyl-1H-indol-4-yl]oxy]acetic acid,

(E) [[3-(2-amino-1,2-dioxoethyl)-1-([1,1′-biphenyl]-4-ylmethyl)-2-methyl-1H-indol-4-yl]oxy]acetic acid,

(F) [[3-(2-amino-1,2-dioxoethyl)-1-[(2,6-dichlorophenyl)methyl]-2-methyl-1H-indol-4-yl]oxy]acetic acid

(G) [[3-(2-amino-1,2-dioxoethyl)-1-[4-(fluorophenyl)methyl]-2-methyl-1H-indol-4-yl]oxy]acetic acid,

(H) [[3-(2-amino-1,2-dioxoethyl)-2-methyl-1-[(1-naphthalenyl)methyl]-1H-indol-4-yl]oxy]acetic acid,

(I) [[3-(2-amino-1,2-dioxoethyl)-2-ethyl-1-(phenylmethyl)-1H-indol-4-yl]oxy]acetic acid,

(J) [[3-(2-amino-1,2-dioxoethyl)-1-[(3-chlorophenyl)methyl]-2-ethyl-1H-indol-4-yl]oxy]acetic acid,

(K) [[3-(2-amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-amino-1,2-dioxoethyl)-2-cyclopropyl-1-(phenylmethyl)-1H-indol-4-yl]oxy]acetic acid,

(N) [[3-(2-amino-1,2-dioxoethyl)-1-([1,1′-biphenyl]-2-ylmethyl)-2-cyclopropyl-1H-indol-4-yl]oxy]acetic acid,

(O) 4-[[3-(2-amino-1,2-dioxoethyl)-2-ethyl-1-(phenylmethyl)-1H-indol-5-yl]oxy]butanoic acid,

(P) 9H-carbazole,

(Q) 9-benzylcarbazole, (AG) 1-(9H-benzylcarbazol-1-halo-4-yloxy-5-alkylamido)alkylacetate,

(AH) 1-(9H-benzylcarbazol-4-yloxy-5-alkylamido) alkylacetate,

(AI) 1-(9H-benzylcarbazol-1-halo-4-yloxy-5-alkylamido) acetic acid,

(AJ) 1-(9H-benzylcarbazol-4-yloxy-5-alkylamido) acetic acid and

(AK) mixtures of (AG) through (AJ) and

(AL) mixtures of (A) through (AK) combined with an additional treatment composition.

Particularly useful prodrugs of the compounds of formula (II) and named compounds (A) thru (AL) are the simple aromatic and aliphatic esters, such as the methyl ester.

The method of the invention also includes treatment of an animal including human, afflicted with sepsis, with a therapeutically effective amount of a sPLA₂ inhibitor within 24 hours after first organ failure, or prevention of sepsis in an animal including human, susceptible to sepsis by administering a sPLA₂ inhibitor compound prior to organ failure or rise in sPLA₂ levels. This method includes administering to said animal in need of such treatment or prevention as described above, a therapeutically effective amount of a composition selected from the group comprising:

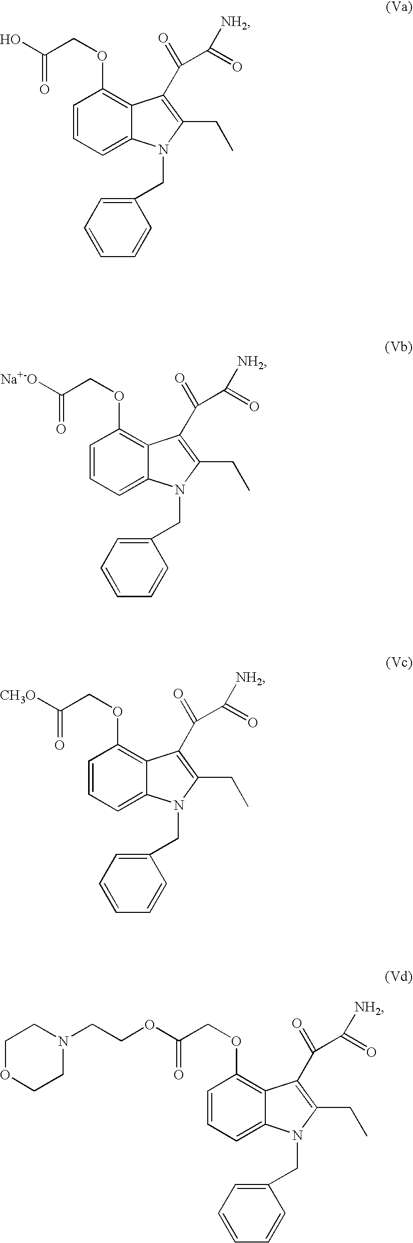

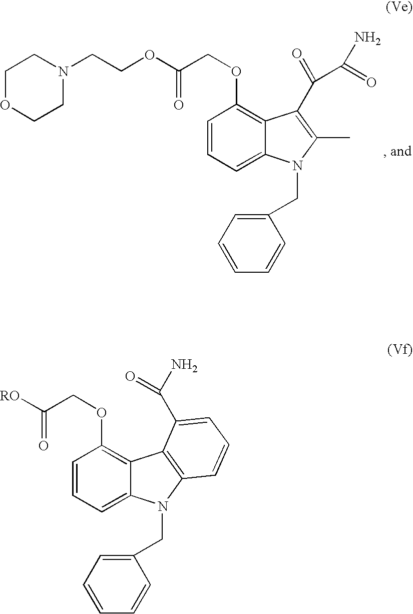

wherein R is methyl, ethyl, sodium ion, or N-morpholinoethyl group.

Preparation of sPLA₂ Inhibitors

The 1H-indole-3-glyoxylamide sPLA₂ inhibitors and method of making them are described in U.S. Pat. No. 5,654,326, the entire disclosure of which is incorporated herein by reference. Another method of making 1H-indole-3-glyoxylamide sPLA₂ inhibitors is described in U.S. patent application Ser. No. 09/105,381, filed Jun. 26, 1998 and titled, “Process for Preparing 4-substituted 1-H-Indole-3-glyoxyamides” the entire disclosure of which is incorporated herein by reference. U.S. patent application Ser. No. 09/105,381 discloses the following process having steps (a) thru (i):

preparing a compound of the formula I or a pharmaceutically acceptable salt or prodrug derivative thereof

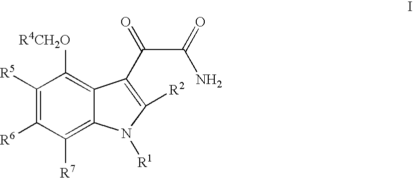

wherein:

R¹ is selected from the group consisting of —C₇-C₂₀ alkyl,

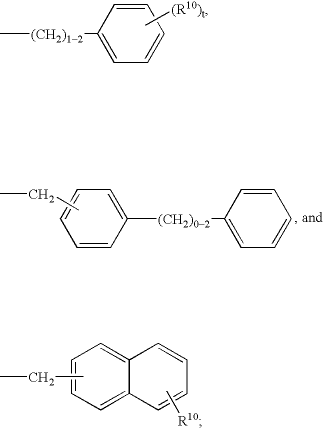

where

R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;

R² is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), aryl, aryloxy and HET;

R⁴ is selected from the group consisting of —CO₂H, SO₃H and —P(O)(OH)₂ or salt and prodrug derivatives thereof; and

R⁵, R⁶ and R⁷ are each independently selected from the group consisting of hydrogen, (C₁-C₆)alkyl, (C₁-C₆)alkoxy, halo(C₁-C₆)alkoxy, halo(C₂-C₆)alkyl, bromo, chloro, fluoro, iodo and aryl;

which process comprises the steps of:

a) halogenating a compound of formula X

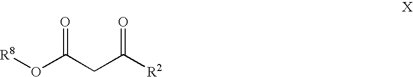

where R⁸ is (C₁-C₆)alkyl, aryl or HET; with SO₂Cl₂ to form a compound of formula IX

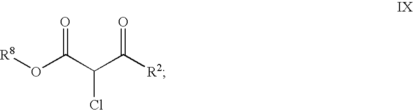

b) Hydrolyzing and decarboxylating a compound of formula IX

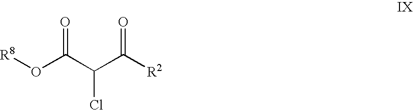

to form a compound of formula VIII

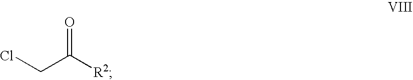

c) alkylating a compound of formula VII

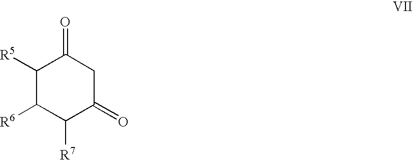

with a compound of formula VIII

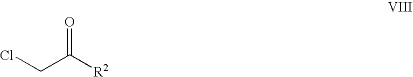

to form a compound of formula VI

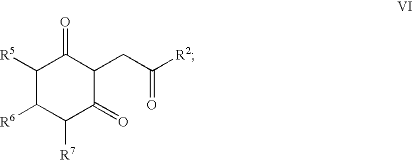

d) aminating and dehydrating a compound of formula VI

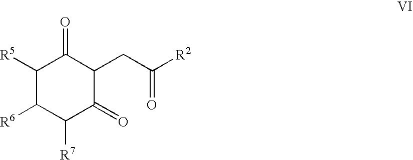

with an amine of the formula R¹NH₂ in the presence of a solvent that forms and azeotrope with water to form a compound of formula V;

e) oxidizing a compound of formula V

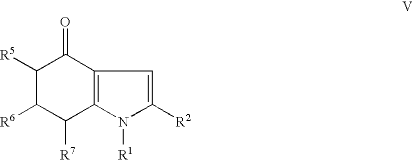

by refluxing in a polar hydrocarbon solvent having a boiling point of at least 150° C. and a dielectric constant of at least 10 in the presence of a catalyst to form a compound of formula IV

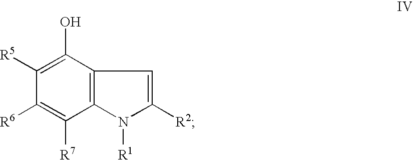

f) alkylating a compound of the formula IV

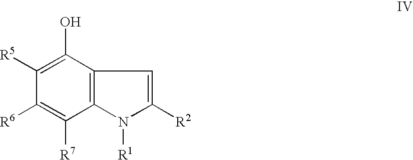

with an alkylating agent of the formula XCH₂R^{4a} where X is a leaving group and R^{4a} is —CO₂R^{4b}, —SO₃R^{4b}, —P(O)(OR^{4b})₂, or —P(O)(OR^{4b})H, where R^{4b} is an acid protecting group to form a compound of formula III

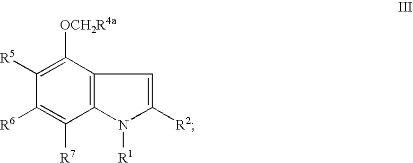

g) reacting a compound of formula III

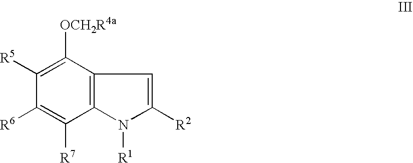

with oxalyl chloride and ammonia to form a compound of formula IIa

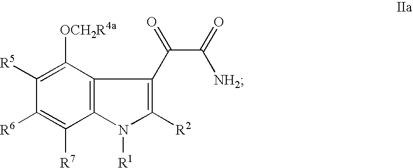

and

h) optionally hydrolyzing a compound of formula IIa

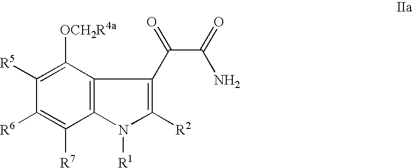

to form a compound of formula I; and

i) optionally salifying a compound of formula I.

The synthesis methodology for making the 1H-indole-3-glyoxylamide sPLA₂ inhibitor starting material may be by any suitable means available to one skilled in the chemical arts. However, such methodology is not part of the present invention which is a method of use, specifically, a method of treating mammal afflicted with or susceptible to sepsis.

The method of the invention is for the prevention of sepsis in a mammal, including a human susceptible to sepsis by clinical determination, or treatment of a mammal, including a human afflicted with sepsis. Said method comprises initiating administration to said mammal, a therapeutically effective amount of the compound represented by formula (Ia), or a pharmaceutically acceptable salt or prodrug derivative thereof, prior to or during an event characterizing sepsis and up to 24 hours after first organ failure;

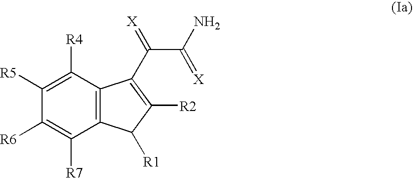

wherein;

Both X are oxygen;

R₁ is selected from the group consisting of

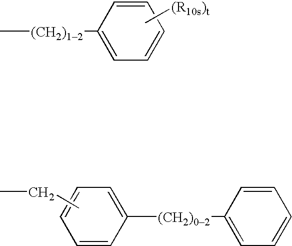

Where R¹⁰ is a radical independently selected from halo, C₁-C₁₀ alkyl, C₁-C₁₀ alkoxy, —S—(C₁-C₁₀ alkyl), and C₁-C₁₀ haloalkyl and t is a number from 0 to 5;

R₂ is selected from the group; halo, cyclopropyl, methyl, ethyl, and propyl;

R₄ and R₅ are independently selected from hydrogen, a non-interfering substituent, or the group, (Lₐ) (acidic group); wherein (Lₐ) is an acid linker; provided, the acid linker group, (Lₐ), for R₄ is selected from the group consisting of;

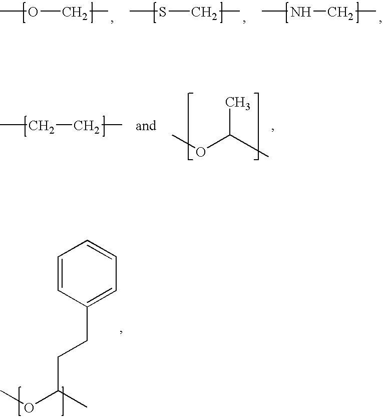

and provided, the acid linker, (Lₐ), for R⁵ is selected from group consisting of;

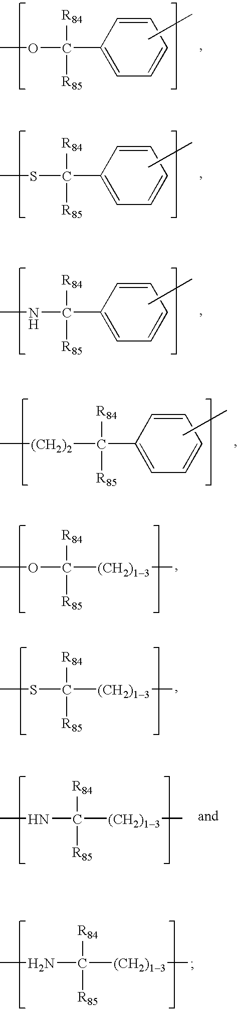

wherein R₈₄ and R₈₅ are each independently selected from hydrogen, C₁-C₁₀-alkyl, aryl, C₁-C₁₀ alkaryl, C₁-C₁₀ aralkyl, carboxy, carbalkoxy, and halo; and

provided, that at least one of R₄ and R₅ must be the group, -(Lₐ)-(acidic group) and wherein the (acidic group) on the group -(Lₐ)-(acidic group) of R₄ or R₅ is selected from —CO₂H, —SO₃H, or —P(O)(OH)₂;

R₆ and R₇ are each independently selected from hydrogen and non-interfering substituents, with the non-interfering substituents being selected from the group consisting of the following: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O(C₁-C₆ alkyl), benzyloxy, phenoxy, phenylthio, —(CONHSO₂R), —CHO, amino, amidino, bromo, carbamyl, carboxyl, carbalkoxy, (CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

Preferred for practicing the method of the invention are 1H-indole-3-glyoxylamide compounds and all corresponding pharmaceutically acceptable salts, solvates and prodrug derivatives thereof which are useful in the method of the invention and include the following:

(A) [[3-(2-Amino-1,2-dioxoethyl)-2-methyl-[(phenylmethyl)-1H-indol-4-yl]oxy]acetic acid,

(B) dl-2-[[3-(2-Amino-1,2-dioxoethyl)-2-methyl-1-(phenylmethyl)-1H-indol-4-yl]oxy]propanoic acid,

(C) [[3-(2-Amino-1,2-dioxoethyl)-1-([1,1′-biphenyl]-2-ylmethyl)-2-methyl-1H-indol-4-yl]oxy]acetic acid,

(D) [[3-(2-Amino-1,2-dioxoethyl)-1-([1,1′-biphenyl]-3-ylmethyl)-2-methyl-1H-indol-4-yl]oxy]acetic acid,

(E) [[3-(2-Amino-1,2-dioxoethyl)-1-([1,1′-biphenyl]-4-ylmethyl)-2-methyl-1H-indol-4-yl]oxy]acetic acid,

(F) [[3-(2-Amino-1,2-dioxoethyl)-1-[(2,6-dichlorophenyl)methyl]-2-methyl-1H-indol-4-yl]oxy]acetic acid

(G) [[3-(2-Amino-1,2-dioxoethyl)-1-[4-(fluorophenyl)methyl]-2-methyl-1H-indol-4-yl]oxy]acetic acid,

(H) [[3-(2-Amino-1,2-dioxoethyl)-2-methyl-1-[(1-naphthalenyl)methyl]-1H-indol-4-yl]oxy]acetic acid,

(I) [[3-(2-Amino-1,2-dioxoethyl)-2-ethyl-1-(phenylmethyl)-1H-indol-4-yl]oxy]acetic acid,

(J) [[3-(2-Amino-1,2-dioxoethyl)-1-[(3-chlorophenyl)methyl]-2-ethyl-1H-indol-4-yl]oxy]acetic acid,

(K) [[3-(2-Amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-Amino-1,2-dioxoethyl)-2-cyclopropyl-1-(phenylmethyl)-1H-indol-4-yl]oxy]acetic acid,

(N) [[3-(2-Amino-1,2-dioxoethyl)-1-([1,1′-biphenyl]-2-ylmethyl)-2-cyclopropyl-1H-indol-4-yl]oxy]acetic acid,

(O) 4-[[3-(2-Amino-1,2-dioxoethyl)-2-ethyl-1-(phenylmethyl)-1H-indol-5-yl]oxy]butanoic acid, and

(P) mixtures of (A) through (P) in any combination.

Included within the definition of pharmaceutically acceptable salts are the relatively non-toxic, inorganic and organic base addition salts of compounds of the present invention, for example, ammonium, quaternary ammonium, and amine cations, derived from nitrogenous bases of sufficient basicity to form salts with the compounds of this invention (see, for example, S. M. Berge, et al., “Pharmaceutical Salts,” J. Phar. Sci., 66: 1-19 (1977)). Moreover, the basic group(s) of the compound of the invention may be reacted with suitable organic or inorganic acids to form salts such as acetate, benzenesulfonate, benzoate, bicarbonate, bisulfate, bitartrate, borate, bromide, camsylate, carbonate, chloride, clavulanate, citrate, chloride, edetate, edisylate, estolate, esylate, fluoride, fumarate, gluceptate, gluconate, glutamate, glycolylarsanilate, hexylresorcinate, bromide, chloride, hydroxynaphthoate, iodide, isothionate, lactate, lactobionate, laurate, malate, malseate, mandelate, mesylate, methylbromide, methylnitrate, methylsulfate, mucate, napsylate, nitrate, oleate, oxalate, palmitate, pantothenate, phosphate, polygalacturonate, salicylate, stearate, subacetate, succinate, tannate, tartrate, tosylate, trifluoroacetate, trifluoromethane sulfonate, and valerate.

Certain compounds of the invention may possess one or more chiral centers, and thus, may exist in optically active forms. Likewise, when the compounds contain an alkenyl or alkenylene group, there exist the possibility of cis- and trans-isomeric forms of the compounds. The R- and S-isomers and mixtures thereof, including racemic mixtures as well as mixtures of cis- and trans-isomers, are contemplated by this invention. Additional asymmetric carbon atoms can be present in a substituent group such as an alkyl group. All such isomers as well as the mixtures thereof are intended to be included in the invention. If a particular stereoisomer is desired, it can be prepared by methods well known in the art by using stereospecific reactions with starting materials which contain the asymmetric centers and are already resolved or, alternatively by methods which lead to mixtures of the stereoisomers and subsequent resolution by known methods. For example, a racemic mixture may be reacted with a single enantiomer of some other compound. This changes the racemic form into a mixture of stereoisomers and diastereomers, because they have different melting points, different boiling points, and different solubilities and can be separated by conventional means, such as crystallization.

Prodrugs are derivatives of the compounds of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility; tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Particularly preferred esters as prodrugs are methyl, ethyl, propyl, isopropyl, n-butyl, isobutyl, tert-butyl, morpholinoethyl, and N,N-diethylglycolamido.

N,N-diethylglycolamido ester prodrugs may be prepared by reaction of the sodium salt of a compound of Formula (I) (in a medium such as dimethylformamide) with 2-chloro-N,N-diethylacetamide (available from Aldrich Chemical Co., Milwaukee, Wis. USA; Item No. 25,099-6). Morpholinylethyl ester prodrugs may be prepared by reaction of the sodium salt of a compound of formula (I) (in a medium such as dimethylformamide) with 4-(2-chloroethyl)morpholine hydrochloride (available from Aldrich Chemical Co., Milwaukee, Wis. USA, Item No. C4, 220-3).

Particularly useful prodrugs of the compounds of formula (I) and named compounds (A) thru (O) are the simple aromatic and aliphatic esters, such as the methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Most preferred in the practice of the method of the invention are the acid, sodium salt, methyl ester, and morpholino-N-ethyl ester forms of [[3-(2-amino-1,2-dioxoethyl)-2-ethyl-1-(phenylmethyl)-1H-indol-4-yl]oxy]acetic acid as represented by the following formulae:

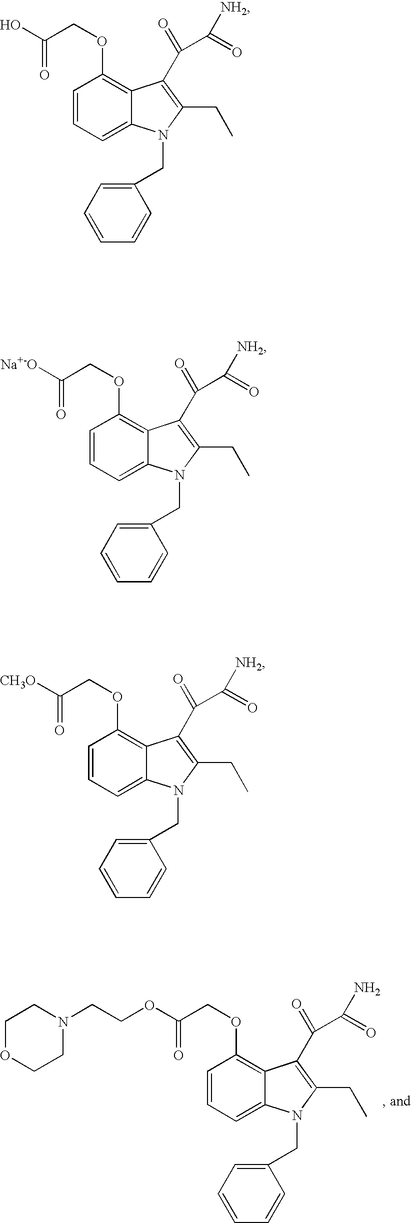

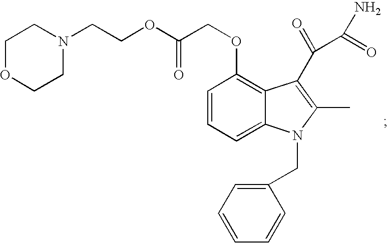

The preparation of which is further described in U.S. provisional patent application S No. 60/063,646 filed Oct. 27, 1997.

Synthesis methods for 1H-indole-3-glyoxylamide sPLA₂ inhibitors are additionally depicted in the following reaction scheme:

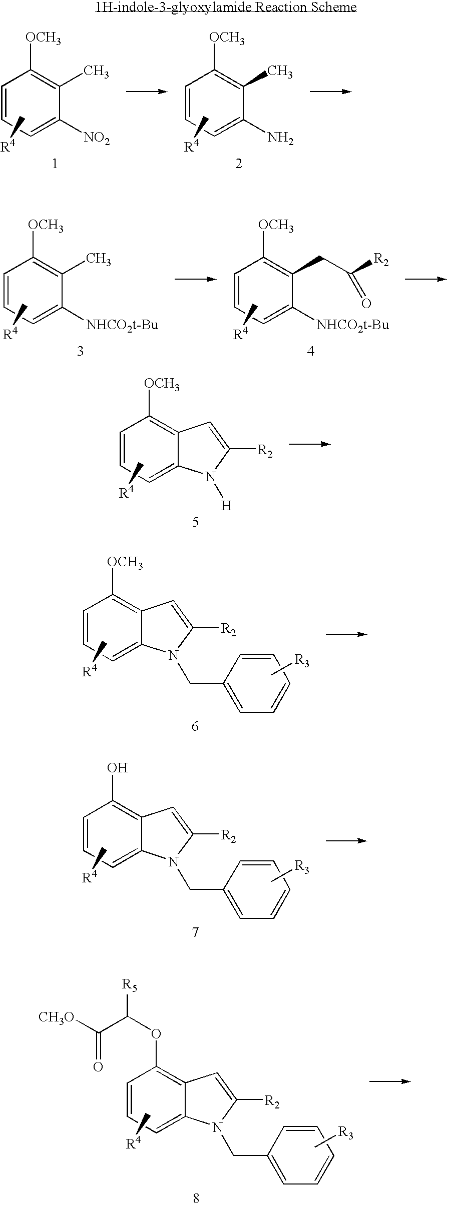

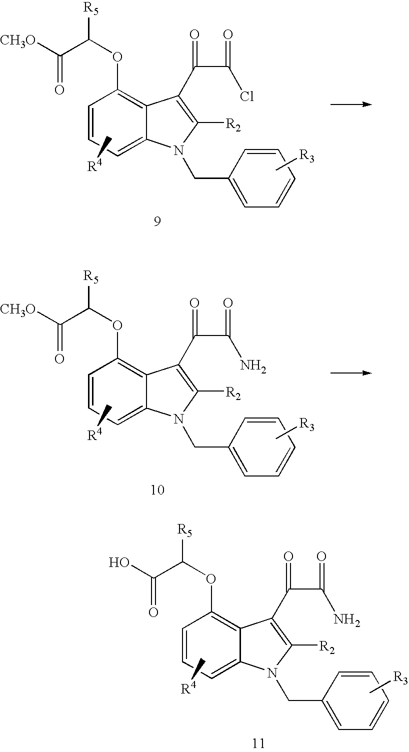

Explanation of Reaction Scheme:

To obtain the glyoxylamides substituted in the 4-position with an acidic function through an oxygen atom, the reactions outlined in scheme 1 are used (for conversions 1 through 5, see Robin D. Clark, Joseph M. Muchowski, Lawrence E. Fisher, Lee A. Flippin, David B. Repke, Michel Souchet, Synthesis, 1991, 871-878, the disclosures of which are incorporated herein by reference. The ortho-nitrotoluene, 1, is readily reduced to the 2-methylaniline, 2, using Pd/C as catalyst. The reduction can be carried out in ethanol or tetrahydrofuran (THF) or a combination of both, using a low pressure of hydrogen. The aniline, 2, on heating with di-tert-butyl dicarbonate in THF at reflux temperature is converted to the N-tert-butylcarbonyl derivative, 3, in good yield. The dilithium salt of the dianion of 3 is generated at −40 to −20° C. in THF using sec-butyl lithium and reacted with the appropriately substituted N-methoxy-N-methylalkanamide. This product, 4, may be purified by crystallization from hexane, or reacted directly with trifluoroacetic acid in methylene chloride to give the 1,3-unsubstituted indole 5. The 1,3-unsubstituted indole 5 is reacted with sodium hydride in dimethylformamide at room temperature (20-25° C.) for 0.5-1.0 hour. The resulting sodium salt of 5 is treated with an equivalent of arylmethyl halide and the mixture stirred at a temperature range of 0-100° C., usually at ambient room temperature, for a period of 4 to 36 hours to give the 1-arylmethylindole, 6. This indole, 6, is O-demethylated by stirring with boron tribromide in methylene chloride for approximately 5 hours (see Tsung-Ying Shem and Charles A Winter, Adv. Drug Res., 1977, 12, 176, the disclosure of which is incorporated herein by reference). The 4-hydroxyindole, 7, is alkylated with an alpha bromoalkanoic acid ester in dimethylformamide (DMF) using sodium hydride as a base, with reactions conditions similar to that described for the conversion of 5 to 6. The α-[(indol-4-yl)oxy]alkanoic acid ester, 8, is reacted with oxalyl chloride in methylene chloride to give 9, which is not purified but reacted directly with ammonia to give the glyoxamide 10. This product is hydrolyzed using 1N sodium hydroxide in methanol. The final glyoxylamide, 11, is isolated either as the free carboxylic acid or as its sodium salt. The sodium salt is a preferred compound of the invention when R₂, R₃, R₄ and R₅ of compound 11, is each a hydrogen atom. When the sodium salt is desired one of skill in the art is aware that the product 10, upon treatment with about 1N NaOH is isolated without further acidic wash.

### The Carbazole and Tetrahydrocarbazole Compounds

Carbazole and tetrahydrocarbazole sPLA₂ inhibitors and methods of making these compounds are set out in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference. The method of the invention includes treatment of a mammal with these compounds.

The method of the invention is for treatment of a mammal, including a human, afflicted with sepsis or susceptible to sepsis. Said method comprises administering to said human prior to and/or up to 24 hours after first organ failure resulting from sepsis, a therapeutically effective amount carbazole or tetrahydrocarbazole represented by the following:

a compound of the formula (Ie)

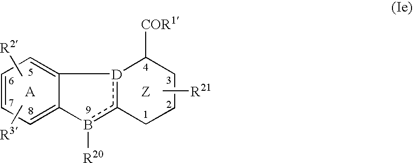

wherein;

a is phenyl or pyridyl wherein the nitrogen is at the 5-, 6-, 7- or 8-position;

one of B or D is nitrogen and the other is carbon; Z is cyclohexenyl, phenyl, pyridyl, wherein the nitrogen is at the 1-, 2-, or 3-position, or a 6-membered heterocyclic ring having one heteroatom selected from the group consisting of sulfur or oxygen at the 1-, 2- or 3-position, and nitrogen at the 1-, 2-, 3- or 4-position;

[symbol] is a double or single bond;

R²⁰ is selected from groups (a), (b) and (c) where;

(a) is —(C₅-C₂₀)alkyl, —(C₅-C₂₀)alkenyl, —(C₅-C₂₀)alkynyl, carbocyclic radicals, or heterocyclic radicals, or

(b) is a member of (a) substituted with one or more independently selected non-interfering substituents; or

(c) is the group -(L)-R⁸⁰; where, -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) and carbon, hydrogen, and oxygen only; and where R⁸⁰ is a group selected from (a) or (b);

R²¹ is a non-interfering substituent;

R¹′ is —NHNH₂, —NH₂ or —CONH₂;

R²′ is selected from the group consisting of —OH, and —O(CH₂)ₜr5′ where

R⁵′ is H, —CN, —NH₂, —CONH₂, —CONR⁹R¹⁰—NHSO₂R¹⁵; —CONHSO₂R¹⁵, where R¹⁵ is —(C₁-C₆)alkyl or —CF₃; phenyl or phenyl substituted with —CO₂H or —CO₂(C₁-C₄)alkyl; and -(Lₐ)-(acidic group), wherein -(Lₐ)- is an acid linker having an acid linker length of 1 to 7 and t is 1-5;

R³′ is selected from non-interfering substituent, carbocyclic radicals, carbocyclic radicals substituted with non-interfering substituents, heterocyclic radicals, and heterocyclic radicals substituted with non-interfering substituents; or a pharmaceutically acceptable racemate, solvate, tautomer, optical isomer, prodrug derivative or salt thereof;

provided that; when R³′ is H, R²⁰ is benzyl and m is 1 or 2; R²′ cannot be —O(CH₂)ₘh; and

Provided that when D is nitrogen, the heteroatom of Z is selected from the group consisting of sulfur or oxygen at the 1-, 2- or 3-position and nitrogen at the 1-, 2-, 3- or 4-position.

Preferred in the practice of the method of the invention are compounds represented by the formula (IIe):

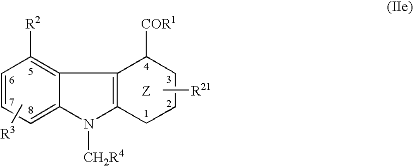

wherein;

Z is cyclohexenyl, or phenyl;

R²¹ is a non-interfering substituent;

R¹ is —NHNH2 or —NH₂;

R² is selected from the group consisting of —OH and O(CH₂)ₘr⁵ where

R⁵ is H, —CO₂H, —CONH₂, —CO₂(C₁-C₄ alkyl);

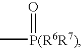

where R⁶ and R⁷ are each independently —OH or —O(C₁-C₄)alkyl; —SO₃H, —SO₃(C₁-C₄ alkyl), tetrazolyl, —CN, —NH₂, —NHSO₂R⁵; —CONHSO₂R¹⁵, where R¹⁵ is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙr⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl; or a pharmaceutically acceptable racemate, solvate, tautomer, optical isomer, prodrug derivative or salt, thereof.

Preferred specific compounds including all salts and prodrug derivatives thereof, for practicing the method of the invention are as follows:

9-benzyl-5,7-dimethoxy-1,2,3,4-tetrahydrocarbazole-4-carboxylic acid hydrazide;

9-benzyl-5,7-dimethoxy-1,2,3,4-tetrahydrocarbazole-4-carboxamide;

[9-benzyl-4-carbamoyl-7-methoxy-1,2,3,4-tetrahydrocarbazol-5-yl]oxyacetic acid sodium salt;

[9-benzyl-4-carbamoyl-7-methoxycarbazol-5-yl]oxyacetic acid;

Methyl [9-benzyl-4-carbamoyl-7-methoxycarbazol-5-yl]oxyacetic acid;

9-benzyl-7-methoxy-5-cyanomethyloxy-1,2,3,4-tetrahydrocarbazole-4-carboxamide;

9-benzyl-7-methoxy-5-(1H-tetrazol-5-yl-methyl)oxy)-1,2,3,4-tetrahydrocarbazole-4-carboxamide;

{9-[(phenyl)methyl]-5-carbamoyl-2-methyl-carbazol-4-yl}oxyacetic acid;

{9-[(3-fluorophenyl)methyl]-5-carbamoyl-2-methyl-carbazol-4-yl}oxyacetic acid;

{9-[(3-methylphenyl)methyl]-5-carbamoyl-2-methyl-carbazol-4-yl}oxyacetic acid;

{9-[(phenyl)methyl]-5-carbamoyl-2-(4-trifluoromethylphenyl)carbazol-4-yl}oxyacetic acid;

9-benzyl-5-(2-methanesulfonamido)ethyloxy-7-methoxy-1,2,3,4-tetrahydrocarbazole-4-carboxamide;

9-benzyl-4-(2-methanesulfonamido)ethyloxy-2-methoxycarbazole-5-carboxamide;

9-benzyl-4-(2-trifluoromethanesulfonamido)ethyloxy-2-methoxycarbazole-5-carboxamide;

9-benzyl-5-methanesulfonamidoylmethyloxy-7-methoxy-1,2,3,4-tetrahydrocarbazole-4-carboxamide;

9-benzyl-4-methanesulfonamidoylmethyloxy-carbazole-5-carboxamide;

[5-carbamoyl-2-pentyl-9-1(phenylmethyl)carbazol-4-yl]oxyacetic acid;

[5-carbamoyl-2-(1-methylethyl)-9-(phenylmethyl)carbazol-4-yl]oxyacetic acid;

[5-carbamoyl-9-(phenylmethyl)-2-[(tri(−1-methylethyl)silyl)oxymethyl]carbazol-4-yl]oxyacetic acid;

[5-carbamoyl-2-phenyl-9-(phenylmethyl)carbazol-4-yl]oxyaceticacid[5-carbamoyl-2-(4-chlorophenyl)-9-(phenylmethyl)carbazol-4-yl]oxyacetic acid;

[5-carbamoyl-2-(2-furyl)-9-(phenylmethyl)carbazol-4-yl]oxyacetic acid;

[5-carbamoyl-9-(phenylmethyl)-2-[(tri(−1-methylethyl)silyl)oxymethyl]carbazol-4-yl]oxyacetic acid, lithium salt;

{9-[(phenyl)methyl]-5-carbamoylcarbazol-4-yl}oxyacetic acid;

{9-[(3-fluorophenyl)methyl]-5-carbamoylcarbazol-4-yl}oxyacetic acid;

{9-[(3-phenoxyphenyl)methyl]-5-carbamoylcarbazol-4-yl}oxyacetic acid;

{9-[(2-Fluorophenyl)methyl]-5-carbamoylcarbazol-4-yl}oxyacetic acid;

{9-[(2-trifluoromethylphenyl)methyl]-5-carbamoylcarbazol-4-yl}oxyacetic acid;

{9-[(2-benzylphenyl)methyl]-5-carbamoylcarbazol-4-yl}oxyacetic acid;

{9-[(3-trifluoromethylphenyl)methyl]-5-carbamoylcarbazol-4-yl}oxyacetic acid;

{9-[(1-naphthyl)methyl]-5-carbamoylcarbazol-4-yl}oxyacetic acid;

{9-[(2-cyanophenyl)methyl]-5-carbamoylcarbazol-4-yl}oxyacetic acid;

{9-[(3-cyanophenyl)methyl]-5-carbamoylcarbazol-4-yl}oxyacetic acid;

{9-[(2-methylphenyl)methyl]-5-carbamoylcarbazol-4-yl}oxyacetic acid;

{9-[(3-methylphenyl)methyl]-5-carbamoylcarbazol-4-yl}oxyacetic acid;

{9-[(3,5-dimethylphenyl)methyl]-5-carbamoylcarbazol-4-yl}oxyacetic acid;

{9-[(3-iodophenyl)methyl]-5-carbamoylcarbazol-4-yl}oxyacetic acid;

{9-[(2-Chlorophenyl)methyl]-5-carbamoylcarbazol-4-yl}oxyacetic acid;

{9-[(2,3-difluorophenyl)methyl]-5-carbamoylcarbazol-4-yl}oxyacetic acid;

{9-[(2,6-difluorophenyl)methyl]-5-carbamoylcarbazol-4-yl}oxyacetic acid;

{9-[(2,6-dichlorophenyl)methyl]-5-carbamoylcarbazol-4-yl}oxyacetic acid;

{9-[(3-trifluoromethoxyphenyl)methyl]-5-carbamoylcarbazol-4-yl}oxyacetic acid;

{9-[(2-biphenyl)methyl]-5-carbamoylcarbazol-4-yl}oxyacetic acid;

{9-[(2-Biphenyl)methyl]-5-carbamoylcarbazol-4-yl}oxyacetic acid;

{9-[(2-Biphenyl)methyl]-5-carbamoylcarbazol-4-yl}oxyacetic acid;

[9-Benzyl-4-carbamoyl-1,2,3,4-tetrahydrocarbaole-5-yl]oxyacetic acid;

{9-[(2-Pyridyl)methyl]-5-carbamoylcarbazol-4-yl}oxyacetic acid;

{9-[(3-Pyridyl)methyl]-5-carbamoylcarbazol-4-yl}oxyacetic acid;

[9-benzyl-4-carbamoyl-8-methyl-1,2,3,4-tetrahydrocarbazol-5-yl]oxyacetic acid;

[9-benzyl-5-carbamoyl-1-methylcarbazol-4-yl]oxyacetic acid;

[9-benzyl-4-carbamoyl-8-fluoro-1,2,3,4-tetrahydrocarbazol-5-yl]oxyacetic acid;

[9-benzyl-5-carbamoyl-1-fluorocarbazol-4-yl]oxyacetic acid;

[9-benzyl-4-carbamoyl-8-chloro-1,2,3,4-tetrahydrocarbazol-5-yl]oxyacetic acid;

[9-benzyl-5-carbamoyl-1-chlorocarbazol-4-yl]oxyacetic acid;

[9-[(Cyclohexyl)methyl]-5-carbamoylcarbazol-4-yl]oxyacetic acid;

[9-[(Cyclopentyl)methyl]-5-carbamoylcarbazol-4-yl]oxyacetic acid;

5-carbamoyl-9-(phenylmethyl)-2-[[(propen-3-yl)oxy]methyl]carbazol-4-yl]oxyacetic acid;

[5-carbamoyl-9-(phenylmethyl)-2-[(propyloxy)methyl]carbazol-4-yl]oxyacetic acid;

9-benzyl-7-methoxy-5-((carboxamidomethyl)oxy)-1,2,3,4-tetrahydrocarbazole-4-carboxamide;

9-benzyl-7-methoxy-5-cyanomethyloxy-carbazole-4-carboxamide;

9-benzyl-7-methoxy-5-((1H-tetrazol-5-yl-methyl)oxy)carbazole-4-carboxamide;

9-benzyl-7-methoxy-5-((carboxamidomethyl)oxy)-carbazole-4-carboxamide; and

[9-Benzyl-4-carbamoyl-1,2,3,4-tetrahydrocarbaole-5-yl]oxyaceticacid

or a pharmaceutically acceptable racemate, solvate, tautomer, optical isomer, prodrug derivative or salt, thereof.

Other desirable carbazole compounds suitable for practicing the method of the invention are selected from those represented by the formula (XXX):

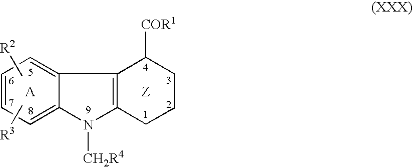

wherein:

R¹ is —NHNH₂, or —NH₂;

R² is selected from the group consisting of —OH and —O(CH₂)ₘr⁵ where

R⁵ is H, —CO₂H, —CO₂(C₁-C₄ alkyl);

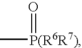

where R⁶ and R⁷ are each independently —OH or —O(C₁-C₄)alkyl; —SO₃H, —SO₃(C₁-C₄ alkyl), tetrazolyl, —CN, —NH₂, —NHSO₂R¹⁵; —CONHSO₂R¹⁵ where R¹⁵ is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙr⁸ where R⁸ is H, CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;

a is phenyl or pyridyl wherein the nitrogen is at the 5-, 6-, 7- or 8-position;

Z is cyclohexenyl, phenyl, pyridyl wherein the nitrogen is at the 1-, 2- or 3-position or a 6-membered heterocyclic ring having one heteroatom selected from the group consisting of sulfur or oxygen at the 1-, 2- or 3-position and nitrogen at the 1-, 2-, 3- or 4-position, or

wherein one carbon on the heterocyclic ring is optionally substituted with ═O; or a pharmaceutically acceptable racemate, solvate, tautomer, optical isomer, prodrug derivative or salt thereof;

provided that one of A or Z is a heterocyclic ring.

Further desirable specific compounds suitable for the method of the invention are selected from the following:

(R, S)— (9-benzyl-4-carbamoyl-1-oxo-3-thia-1,2,3,4-tetrahydrocarbazol-5-yl)oxyacetic acid; (R,S)-(˜9-benzyl-4-carbamoyl-1-oxo-3-thia-1,2,3,4-tetrahydrocarbazol-5-yl)oxyacetic acid; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-beta-carbolin-5-yl)oxy]acetic acid or a pharmaceutically acceptable racemate, solvate, tautomer, optical isomer, prodrug derivative or salt thereof.

Particularly preferred carbazole type compounds for the treatment and/or prevention of sepsis are represented by the formulae (Xe) and (xie) below:

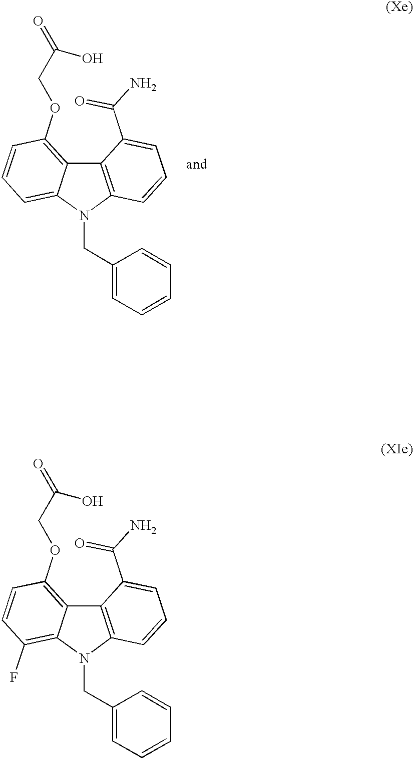

For all of the above compounds of the carbazole or tetrahydrocarbazole type it is advantageous to use them in their (i) acid form, or (ii) pharmaceutically acceptable (e.g., Na, K) form, or (iii) and prodrugs derivatives (e.g., Methyl ester, ethyl ester, n-butyl ester, morpholino ethyl ester).

Prodrugs are derivatives of sPLA₂ inhibitors used in the method of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Specific preferred prodrugs are ester prodrugs inclusive of methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Carbazole and tetrahydrocarbazole sPLA₂ inhibitor compounds useful for practicing the method of the invention may be made by the following general methods:

the compounds of formula Ie where Z is cyclohexene are prepared according to the following reaction Schemes Ig(a) and (c).

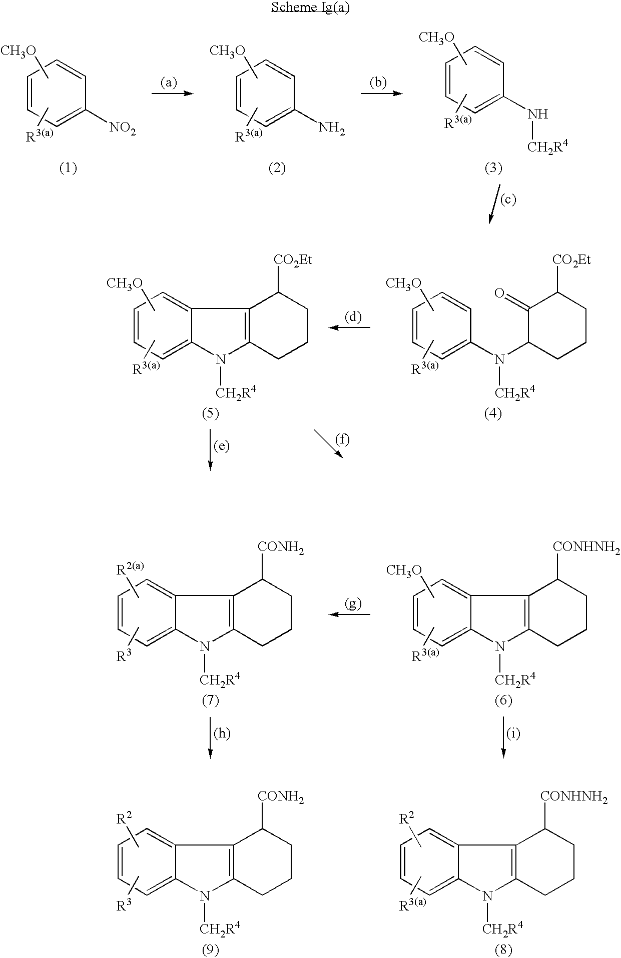

wherein;

R¹ is —NH₂, R^{3(a)} is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁₀-C₆)alkenyl; or —(CH₂)ₙr⁸ where R⁸ is H, —CONH₂, —NR⁹R¹⁰, —CN or phenyl where R⁹ and R¹⁰ are independently hydrogen, —CF₃, phenyl, -(C₁-C₄)alkyl, —(C₁-C₄)alkylphenyl or -phenyl(C₁-C₄)alkyl and n is 1 to 8;

when R¹ is —NHNH₂, R^{3(a)} is H, —O(C₁-C₄)alkyl, halo, —(C₁-C₆)alkyl, phenyl, —(C₁-C₄)alkylphenyl; phenyl substituted with —(C₁-C₆)alkyl, halo or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙr⁸ where R⁸ is H, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

R^{2(a)} is —OCH₃ or —OH.

An appropriately substituted nitrobenzene (1) can be reduced to the aniline (2) by treatment with a reducing agent, such as hydrogen in the presence of Pd/C, preferably at room temperature.

Compound (2) is N-alkylated at temperatures of from about 0 to 20° C. using an alkylating agent such as an appropriately substituted aldehyde and sodium cyanoborohydride to form (3). Alternately, an appropriately substituted benzyl halide may be used for the first alkylation step. The resulting intermediate is further N-alkylated by treatment with 2-carbethoxy-6-bromocyclohexanone, preferably at temperatures of about 80° C. to yield (4) or by treatment with potassium hexamethyldisilazide and the bromoketoester.

The product (4) is cyclized to the tetrahydrocarbazole (5) by refluxing with zncl₂ in benzene for from about 1 to 2 days, preferably at 80° C. (see Julia, M.; Lenzi, J. Preparation d'acides tetrahydro-1,2,3,4-carbazole-1 ou-4. Bull.Soc.Chim.France, 1962, 2262-2263). Compound (5) is converted to the hydrazide (6) by treatment with hydrazine at temperatures of about 100° C., or to the amide (7) by reacting with methylchloroaluminum amide in benzene (see Levin, J. I.; Turos, E.; Weinreb, S. M. An alternative procedure for the aluminum-mediated conversion of esters to amides. Syn.Comm., 1982, 12, 989-993). Alternatively, (7) may be produced by treatment of (6) with Raney nickel active catalyst.

It will be readily appreciated that when R^{3(a)} is:

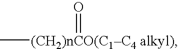

Conversion to the amide will also be achieved in this procedure.

Compounds (6) and (7) may be dealkylated, preferably at 0° C. to room temperature, with a dealkylating agent, such as boron tribromide or sodium thioethoxide, to give compound (7) where R^{2(a)} is —OH, which may then be further converted to compound (9), by realkylating with a base, such as sodium hydride, and an alkylating agent, such as Br(CH₂)ₘr⁵, where R⁵ is the carboxylate or phosphonic diester or nitrile as defined above. Conversion of R² to the carboxylic acid may be accomplished by treatment with an aqueous base. When R² is nitrile, conversion to the tetrazole may be achieved by reacting with tri-butyl tin azide or conversion to the carboxamide may be achieved by reacting with basic hydrogen peroxide. When R² is the phosphonic diester, conversion to the acid may be achieved by reacting with a dealkylating agent such as trimethylsilyl bromide. The monoester may be accomplished by reacting the diester with an aqueous base.

When R² and R³ are both methoxy, selective demethylation can be achieved by treating with sodium ethanethiolate in dimethylformamide at 100° C.

An alternative synthesis of intermediate (5) is shown in Scheme I(b), as follows.

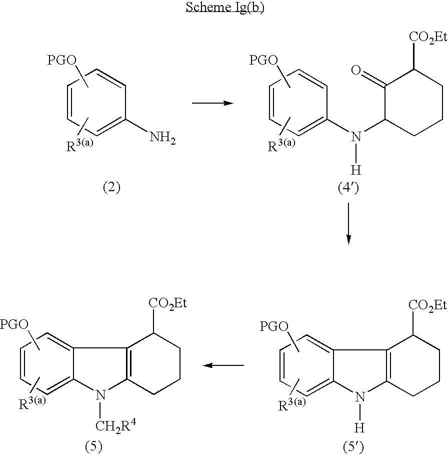

where PG is a protecting group;

R^{3a} is as defined in Scheme 1, above. The aniline (2) is N-alkylated with 2-carbethoxy-6-bromocyclohexanonein dimethyl formamide in the presence of sodium bicarbonate for 8-24 hours at 50° C. Preferred protecting groups include methyl, carbonate, and silyl groups, such as t-butyldimethylsilyl. The reaction product (4′) is cyclized to (5′) using the zncl₂ in benzene conditions described in Scheme I(a), above. N-alkylation of (5′) to yield (5) is accomplished by treatment with sodium hydride and the appropriate alkyl halide in dimethylformamide at room temperature for 4-8 hours.

It is an aspect of this invention that the compound of formula (I) (Ie), or (II) is highly bioavailable upon oral administration, and when administered prior to the onset of a rise in sPLA₂ levels or within 24 hours after failure, is effective against the effects of sepsis or septic shock thereby preventing organ failure and/or death.

Specifically, it has been observed in a clinical trial that a reduction in mortality is achieved when a subgroup population of clinical trial participants was administered a compound of formula I (i.e. compound (Vb)) within 24 hours, preferably within 18 hours, more preferably within 12 after first organ failure and continuing for up to 7 days thereafter. The mortality reduction was observed to be higher, the sooner the administration of a compound of formula I was initiated after first organ failure.

The compound of formula I, Ie or II is believed to reduce mortality due to sepsis when administered to a patient susceptible to sepsis or expected to undergo procedures that predispose such patient to sepsis, before sPLA₂ levels rise (i.e. preventative), or within 24 hours but preferably within 18 hours after first organ failure, when sPLA₂ levels are high or on the rise (treatment). The effectiveness appears to decrease when sPLA₂ levels have peaked (typically about after about 24 after first organ failure) or are on the decline. This observation may possibly indicate that the body's defense mechanisms have been overwhelmed in which case the patients typically die or that the patient's own defense mechanism have suppressed the rising sPLA₂ levels. The later patients typically survive and are believed to be less in need (less sick) of sPLA₂ inhibitor compounds after about 24 hours. The mechanism(s) or explanations for occurrences or observations are not aspects of this invention.

Synthesis of the Compounds of the Invention:

The synthesis of ((3-(2-amino-1,2-dioxoethyl)-2-methyl-1-(phenylmethyl)-1H-indol-4-yl)oxy)acetic acid, N-morpholino ethyl ester (compound of formula I, supra.) uses as starting material ((3-(2-amino-1,2-dioxoethyl)-2-methyl-1-(phenylmethyl)-1H-indol-4-yl)oxy)acetic acid, or a salt thereof. This starting material may be prepared by the reaction schemes or method of Example 1 of U.S. Pat. No. 5,654,326 (the disclosure of which is incorporated herein by reference). Similar methods are shown in European Patent Application No. 95302166.4, Publication No. 0 675 110 (publ., 4 Oct. 1995). Other conventional methods may also be used for preparing the starting material. Procedures useful for the synthesis of the compound of this invention are specified in Example 1 set out below:

### EXAMPLE 1

Preparation of ((3-(2-Amino-1,2-dioxoethyl)-2-methyl-1-(phenylmethyl)-1H-indol-4-yl)oxy)acetic acid, N-morpholino ethyl ester, a compound represented by the formula:

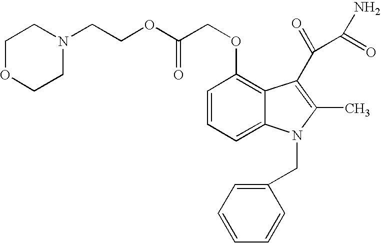

Part A. Preparation of N-tert-butoxycarbonyl-3-methoxy-2-methylaniline.

A solution of 44.4 g (344 mmol) of 3-methoxy-2-methylaniline and 75 g (344 mmol) of di-tert-butyl dicarbonate in 400 mL of THF was heated to maintain reflux for 4 hours. After concentrating at reduced pressure, the residue was taken up in ethyl acetate, washed with 1N citric acid, water and dried (MgSO₄). After removing the solvent at reduced pressure, the residue was crystallized from hexane to give 64.5 g (84% yield) of N-tert-butoxycarbonyl-3-methoxy-2-methylaniline, mp, 56-57° C.

Analysis for C₁₃H₁₉NO₃:

Calculated: C, 65.80; H, 8.07; N, 5.90

Found: C, 63.32; H, 7.83; N, 5.56.

Part B. Preparation of 4-Methoxy-2-methyl-1H-indole.

A solution of 280 mL (0.36 mol) of 1.3M sec-butyl lithium in cyclohexane was added slowly to N-tert-butoxycarbonyl-3-methoxy-2-methylaniline (43 g, 0.18 mol) in 300 mL of THF keeping the temperature below −40° C. with a dry ice-ethanol bath. The bath was removed and the temperature allowed to rise to −20° C. and then the bath replaced. After the temperature had cooled to −60° C., 18.5 g (0.18 mol) of N-methoxy-N-methylglyoxylamide in an equal volume of THF was added dropwise. The reaction mixture was stirred 1 hour, the cooling bath removed and stirred an additional 1 hour. It was then poured into a mixture of 600 mL of ether and 600 mL of 1N HCl. The organic layer was separated, washed with water, dried over MgSO₄, and concentrated at reduced pressure to give 39.5 g of a mixture of 1-(2-(tertbutoxycarbonylamino)-6-methoxyphenyl)-2-propanone and starting anilide. This mixture was dissolved in 100 mL of methylene chloride and 40 mL of trifluoroacetic acid and stirred for a total of 26 hours. The mixture was washed with water, dried (MgSO₄) and concentrated at reduced pressure. The residue was chromatographed on silica gel eluting with 20% EtOAc/hexane to give on crystallization from CH₂-C₁₂/hexane 13.9 g of 4-methoxy-2-methyl-1H-indole, mp, 80-86° C.

Analysis for C₁₀H₁₁NO:

Calculated: C, 74.51; H, 6.88; N, 8.69

Found: C, 74.41; H, 7.08; N, 8.47.

Part C. Preparation of 4-Methoxy-2-methyl-1-(phenylmethyl)-1H-indole.

4-Methoxy-2-methyl-1H-indole (1 g, 6.2 mmol) was added to 248 mg (6.2 mmol) of 60% sodium hydride/mineral oil (washed with hexane before adding DMF) in 15 mL of DMF and after stirring for 0.5 hour, 0.74 mL (6.2 mmol) of benzyl bromide was added. The mixture was stirred at room temperature for 18 hours, diluted with water and extracted with ethyl acetate. The ethyl acetate solution was washed with brine, dried (MgSO₄) and after concentrating at reduced pressure, the residue was chromatographed on silica gel eluting with 20% EtOAc/hexane to give 1.3 g (84% yield) of 4-methoxy-2-methyl-1-(phenylmethyl)-1H-indole, melting at 96-116° C.

Analyses for C₁₇H₁₇NO:

Calculated: C, 81.24; H, 6.82; N, 5.57

Found: C, 81.33; H, 6.74; N, 5.29.

Part D. Preparation of 4-Hydroxy-2-methyl-1-(phenylmethyl)-1H-indole.

A solution of 1.25 g (5 mmol) of 4-methoxy-2-methyl-1-(phenylmethyl)-1H-indole and 20 mL of 1M BBr₃/CH₂Cl₂ in 50 mL of methylene chloride was stirred at room temperature for 5 hours and concentrated at reduced pressure. The residue was dissolved in ethyl acetate, washed with brine and dried (MgSO₄). After concentrating at reduced pressure, the residue was chromatographed on silica gel eluting with 20% EtOAc/hexane to give 577 mg (49% yield) of 4-hydroxy-2-methyl-1-(phenylmethyl)-1H-indole, 125-127° C.

Analyses for C₁₆H₁₅NO:

Calculated: C, 80.98; H, 6.37; N, 5.90

Found: C, 80.76; H, 6.26; N, 5.80.

Part E. Preparation of ((2-Methyl-1-(phenylmethyl)-1H-indol-4-yl)oxy)acetic acid methyl ester.

4-Hydroxy-2-methyl-1-(phenylmethyl)-1H-indole (530 mg, 2.2 mmol) was added to 88 mg (2.2 mmol) of 60% NaH/mineral oil in 20 mL of DMF and the mixture stirred for 0.67 hours. Then, 0.21 mL (2.2 mmol) of methyl bromoacetate was added and stirring maintained for 17 hours. The mixture was diluted with water and extracted with ethyl acetate. The ethyl acetate solution was washed with brine, dried (MgSO₄), and concentrated at reduced pressure. The residue was chromatographed on silica gel eluting with 20% EtOAc/hexane to give 597 mg (88% yield) of ((2-methyl-1-(phenylmethyl)-1H-indol-4-yl)oxy)acetic acid methyl ester, 140-143° C.

Analyses for C₁₉H₁₉NO₃:

Calculated: C, 73.77; H, 6.19; N, 4.53

Found: C, 74.01; H, 6.23; N, 4.32.

Part F. Preparation of ((3-(2-Amino-1,2-dioxoethyl)-2-methyl-1-(phenylmethyl)-1H-indol-4-yl)oxy)acetic acid methyl ester.

Oxalyl chloride (0.16 mL, 1.9 mmol) was added to 582 mg (1.9 mmol) of ((2-methyl-1-(phenylmethyl)-1H-indol-4-yl)oxy)acetic acid methyl ester in 10 mL of methylene chloride and the mixture stirred for 1.5 hours. The mixture was concentrated at reduced pressure and residue taken up in 10 mL of methylene chloride. Anhydrous ammonia was bubbled in for 0.25 hours, the mixture stirred for 1.5 hours and evaporated at reduced pressure. The residue was stirred with 20 mL of ethyl acetate and the mixture filtered. The filtrate was concentrated to give 672 mg of a mixture of ((3-(2-amino-1,2-dioxoethyl)-2-methyl-1-(phenylmethyl)-1H-indol -4-yl)oxy)acetic acid, methyl ester and ammonium chloride, mp. 202-215° C.

Part G. Preparation of ((3-(2-Amino-1,2-dioxoethyl)-2-methyl-1-(phenylmethyl)-1H-indol-4-yl)oxy)acetic acid.

A mixture of 660 mg (1.7 mmol) of ((3-(2-amino-1,2-dioxoethyl)-2-methyl-1-(phenylmethyl)-1H-indol-4-yl)oxy)acetic acid methyl ester and 10 mL of 1N NaOH in 30 mL of methanol was heated to maintain reflux for 1 hour, cooled to room temperature and stirred for 0.5 hour. The mixture was concentrated at reduced pressure and the residue taken up in EtOAc/water. The aqueous layer was separated, made acidic to pH 2-3 with 1N HCl and extracted with EtOAc. On concentrating the EtOAc solution, 431 mg (69% yield) of ((3-(2-amino-1,2-dioxoethyl)-2-methyl-1-(phenylmethyl)-1H-indol-4-yl)oxy)acetic acid crystallized, melting at 218-220° C.

Analyses for C₂₀H₁₈N₂O₅:

Calculated: C, 65.57; H, 4.95; N, 7.65

Found: C, 63.31; H, 4.79; N, 6.91.

Part G2. Preparation of ((3-(2-Amino-1,2-dioxoethyl)-2-methyl-1-(phenylmethyl)-1H-indol-4-yl)oxy)acetic acid Sodium Salt.

A mixture of 660 mg (1.7 mmol) of ((3-(2-amino-1,2-dioxoethyl)-2-methyl-1-(phenylmethyl)-1H-indol-4-yl)oxy)acetic acid methyl ester and 10 mL of 1N NaOH in 30 mL of methanol was heated to maintain reflux for 1 hour, cooled to room temperature and stirred for 0.5 hour. The mixture was concentrated at reduced pressure and the residue taken up in EtOAc/water. The aqueous layer was separated, and concentrated to dryness preferably under vacuum. The product is optionally washed with an aprotic solvent e.g. hexane and further dried to afford target compound.

Part H. Preparation of [[3-(2-Amino-1,2-dioxoethyl)-1-phenyl-3-ylmethyl)-2-methyl-1H-indol-4-yl)oxy)acetic acid, N-morpholino ethyl ester.

The compound of the present invention may be formed by the reaction of 4-(2-chloroethyl)morpholine hydrochloride (available from Aldrich Chemical Co., Milwaukee, Wis. USA, Item No. C4, 220-3) and suitable base preferably CsCO₃; and ((3-(2-amino-1,2-dioxoethyl)-2-methyl-1-(phenylmethyl)-1H-indol-4-yl)oxy)acetic acid, sodium salt in a suitable solvent, preferably dimethylformamide. The slurry should be heated to 60° C. or other appropriate temperature until a solution is formed. Heating should continued until the reaction is complete. The reaction mixture should be worked up to isolate the product using conventional organic laboratory techniques.

### EXAMPLE 2

Preparation of 2-Ethyl-4-methoxy-1H-indole.

A solution of 140 ml (0.18 mol) of 1.3M sec-butyl lithium in cyclohexane is added slowly to N-tert-butoxycarbonyl-3-methoxy-2-methylaniline (21.3 g, 0.09 mol) in 250 ml of THF keeping the temperature below −40° C. with a dry ice-ethanol bath. The bath is removed and the temperature allowed to rise to 0° C. and then the bath replaced. After the temperature has cooled to −60° C., 18.5 g (0.18 mmol) of N-methoxy-N-methylpropanamide in an equal volume of THF is added dropwise. The reaction mixture is stirred 5 minutes, the cooling bath removed and stirred an additional 18 hours. It is then poured into a mixture of 300 ml of ether and 400 ml of 0.5N HCl. The organic layer is separated, washing with water, brine, dried over MgSO₄, and concentrated at reduced pressure to give 25.5 g of a crude of 1-[2-(tert-butoxycarbonylamino)-6-methoxyphenyl]-2-butanone. This material is dissolved in 250 ml of methylene chloride and 50 ml of trifluoroacetic acid and stirred for a total of 17 hours. The mixture is concentrated at reduced pressure and ethyl acetate and water added to the remaining oil. The ethyl acetate is separated, washed with brine, dried (MgSO₄) and concentrated. The residue is chromatographed three times on silica eluting with 20% EtOAc/hexane to give 13.9 g of 2-ethyl-4-methoxy-1H-indole.

Analysis for C₁₁H₁₃NO:

Calculated: C, 75.40; H, 7.48; N, 7.99;

Found: C, 74.41; H, 7.64; N, 7.97.

Part B. Preparation of 2-Ethyl-4-methoxy-[(phenylmethyl)-1H-indole.

2-Ethyl-4-methoxy-1H-indole (4.2 g, 24 mmol) is dissolved in 30 ml of DMF and 960 mg (24 mmol) of 60% NAH/mineral oil is added. After 1.5 hours, 2.9 ml (24 mmol) of benzyl bromide is added. After 4 hours, the mixture is diluted with water extracting twice with ethyl acetate. The combined ethyl acetate is washed with brine, dried (MgSO₄) and concentrated at reduced pressure. The residue is chromatographed on silica gel and eluted with 20% EtOAc/hexane to give 3.1 g (49% yield) of 2-ethyl-4-methoxy]-(phenylmethyl)-1H-indole.

Part C. Preparation of 2-Ethyl-4-hydroxy-[(phenylmethyl)-1H-indole.

A solution of 3.1 g (11.7 mmol) of 2-ethyl-4-methoxy-1-(phenylmethyl)-1H-indole and 48.6 ml of 1M BBr₃/CH₂Cl₂ in 50 ml of methylene chloride is stirred at room temperature for 5 hours and concentrated at reduced pressure. The residue is dissolved in ethyl acetate, washed with brine and dried (Mgso₄). After concentrating at reduced pressure, the residue is chromatographed on silica gel eluting with 20% EtOAc/hexane to give 1.58 g (54% yield) of 2-ethyl-4-hydroxyl-(phenylmethyl)-1H-indole, mp, 86-90° C.

Analysis for C₁₇H₁₇N₀:

Calculated: C, 81.24; H, 6.82; N, 5.57;

Found: C, 81.08; H, 6.92; N, 5.41.

Part D. Preparation of [[2-Ethyl-1-(phenylmethyl)-1H-indol-4-yl]oxy]acetic acid tert-butyl ester.

2-Ethyl-4-hydroxy-1-(phenylmethyl)-1H-indole (5.82 g, 20 mmol) is added to 7.82 g (24 mmol) cesium carbonate in 25 ml DMF and the mixture is stirred at 35° C. for 30 minutes. After cooling to 20° C., a solution of tert-butyl bromoacetate (4.65 g, 23.8 mmol) in 5 ml DMF is added and stirring maintained until the reaction is judged complete by TLC analysis (several hours). The mixture is diluted with water and extracted with ethyl acetate. The ethyl acetate solution is washed with brine, dried (MgSO₄) and concentrated at reduced pressure to give 6.8 g of solid.

Mass spectrum: 365

Analyses for C₂₃H₂₇NO₃:

Calculated: C, 75.59; H, 7.75; N, 3.83;

Found: C, 75.87; H, 7.48; N, 3.94.

Part E. Preparation of [[2-Ethyl-1-(phenylmethyl)-3-ureido-1H-indol-4-yl]oxy]acetic acid tert-butyl ester.

A solution of 2.3 g (6.3 mmol) [[2-ethyl-1-(phenylmethyl)-1H-indol-4-yl]oxy]acetic acid tert-butyl ester and 4.8 g (12.6 mmol) bis(2,2,2-trichloroethyl)-azodicarboxylate in diethyl ether is stirred for 24 hours at room temperature. The resulting solid is filtered and vacuum dried. This adduct (1 g, 1.3 mmol) is dissolved in 10 ml of THF and treated with zinc (1 g) and glacial acetic acid (0.5 ml). After stirring for 30 minutes at room temperature an excess of trimethylsilylisocyanate in 1 ml of THF is added and stirring is continued at room temperature for 18 hours. The mixture is diluted with water and extracted with ethyl acetate. The organic layer is washed with brine, dried over MgSO₄ and concentrated to dryness to give 0.385 g (69% yield) of the subtitled material.

Mass spectrum: 423.

Analyses for C₂₄H₂₉N₃O₄:

Calculated: C, 68.07; H, 6.90; N, 9.92;

Found: C, 67.92; H, 6.84; N, 9.70.

Part F. Preparation of [[3-(2-Amino-1,2-dioxoethyl)-2-ethyl-1-(phenylmethyl)-1H-indol-4-yl]oxy]acetic acid.

A mixture of 788 mg (2 mmol) of [3-(2-amino-1,2-dioxoethyl)-2-ethyl-1-(phenylmethyl)-1H-indol-4-yl]oxy]-acetic acid methyl ester, 10 ml of ln NaOH and 30 ml of methanol is heated to maintain reflux for 0.5 hour, stirred at room temperature for 0.5 hour and concentrated at reduced pressure. The residue is taken up in ethyl acetate and water, the aqueous layer separated and made acidic to pH 2-3 with 1N HCl. The precipitate is filtered and washed with ethyl acetate to give 559 mg (74% yield) of ([3-(2-amino-1,2-dioxoethyl)-2-ethyl-1-(phenylmethyl)-1H-indol-4-yl]oxy]acetic acid, mp, 230-234° C.

Analyses for C₂₁H₂₀N₂O₅:

Calculated: C, 65.96; H, 5.80; N, 7.33;

Found: C, 66.95; H, 5.55; N, 6.99.

### EXAMPLE 3

Preparation of [[3-(2-Amino-1,2-dioxoethyl)-1-([1,1′-biphenyl]-2-ylmethyl)-2-methyl-1H-indol-4-yl]oxy]acetic acid, a compound represented by the formula:

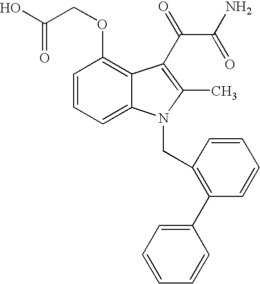

Part A. Preparation of 1-([1,1′-Biphenyl]-2-ylmethyl)-4-methoxy-2-methyl-1H-indole.

Using the procedure described in Example 1, Part C, 1 g (6.2 mmol) of 4-methoxy-2-methyl-1H-indole was reacted with 248 mg (6.2 mmol) of 60% NaH/mineral oil and then 1.1 mL (6.2 mmol) of 2-(bromomethyl)biphenyl to give after chromatography on silica (eluting with 17% EtOAc/hexane) 1.63 g (80% yield) of 1-([1,1′-biphenyl]-2-ylmethyl)-4-methoxy-2-methyl-1H-indole as an oil.

Analyses for C₂₃H₂₁NO:

Calculated: C, 84.37; H, 6.46; N, 4.28

Found: C, 84.11; H, 5.66; N, 3.83.

Part B. Preparation of 1-([1,1′-Biphenyl]-2-ylmethyl)-4-hydroxy-2-methyl-1H-indole.

By the method used in Example 1, Part D, 1.6 g (4.9 mmol) of 1-([1,1′-biphenyl]-2-ylmethyl)-4-methoxy-2-methyl-1H-indole was O-demethylated by treating it with 20 mL of 1M BBr₃/CH₂Cl₂. The crude product was chromatographed on silica gel and eluted with 20% EtOAc/hexane to give 841 mg (55% yield) of 1-([1,1′-biphenyl]-2-ylmethyl)-4-hydroxy-2-methyl-1H-indole.

Analyses for C₂₂H₁₉NO:

Calculated: C, 84.32; H, 6.11; N, 4.47

Found: C, 84.59; H, 6.33; N, 4.75.

Part C. Preparation of [[1-([1,1′-Biphenyl]-2-ylmethyl)-2-methyl-1H-indol-4-yl]oxy]acetic acid methyl ester.

1-([1,1′-Biphenyl]-2-ylmethyl)-4-hydroxy-2-methyl-1H-indole (767 mg, 2.45 mmol) was alkylated by treating with 0.23 mL (2.45 mmol) of methyl bromoacetate and 98 mg (2.45 mmol) of 60% NaH/mineral oil in DMF as described in Example 1, Part E. The product was purified by chromatography over silica gel eluting with 20% EtOAc/hexane, to give 730 mg (77% yield) of [[1-([1,1′-biphenyl]-2-ylmethyl)-2-methyl-1H-indol-4-yl]oxy]acetic acid methyl ester, 99-101° C.

Analyses for C₂₅H₂₃NO₃:

Calculated: C, 77.90; H, 6.01; N, 3.63

Found: C, 78.11; H, 6.17; N, 3.74.

Part D. Preparation of [[3-(2-Amino-1,2-dioxoethyl)-[([1,1′-biphenyl]-2-ylmethyl)-2-methyl-1H-indol-4-yl]oxy]acetic acid methyl ester.

Using the procedure in Example 1, Part F, 715 mg (1.9 mmol) of [[1-([1,1′-biphenyl]-2-ylmethyl)-2-methyl-1H-indol-4-yl]oxy]acetic acid methyl ester was reacted first with 0.16 mL (1.9 mmol) of oxalyl chloride and then excess ammonia to give a white solid. This was stirred with ethyl acetate and the insoluble material separated and dried to give 660 mg of a mixture of [-[3-(2-amino-1,2-dioxoethyl)-1-([1,1′-biphenyl]-2-ylmethyl)-2-methyl-1H-indol-4-yl]oxy]acetic acid methyl ester and ammonium chloride. This mixture melted at 144-148° C.

Part E. Preparation of [[3-(2-Amino-1,2-dioxoethyl)-1-([1,11-biphenyl]-2-ylmethyl)-2-methyl-1H-indol-4-yl]oxy]acetic acid.

A mixture of 648 mg (1.4 mmol) of [[3-(2-amino-1,2-dioxoethyl)-1-([1,11-biphenyl]-2-ylmethyl)-2-methyl-1H-indol-4-yl]oxy]acetic acid methyl ester in 10 mL of 1N NaOH and 20 mL of MeOH was heated to maintain reflux for 1 hour, cooled to room temperature and stirred 0.5 hour. The mixture was concentrated, the residue stirred with a mixture of EtOAc/water and the solid material that did not dissolve was filtered and dried to give 227 mg (35% yield) of [[3-(2-amino-1,2-dioxoethyl)-1-([1,1′-biphenyl]-2-ylmethyl)-2-methyl-1H-indol-4-yl]oxy]acetic acid, sodium salt, mp, >265° C.

Analyses for C₂₆H₂₁N₂O₅Na:

Calculated: C, 67.24; H, 4.56; N, 6.03

Found: C, 69.38; H, 4.88; N, 5.42.

Part F. The aqueous layer was separated from the filtrate from above and made acidic to pH 2-3 with 1N HCl. The precipate was extracted with EtOAc and upon concentrating the EtOAc, 128 mg (20% yield) of ([3-(2-amino-1,2-dioxoethyl)-1-([1,1′-biphenyl]-2-ylmethyl)-2-methyl-1H-indol-4-yl]oxy]acetic acid precipitated, mp, 228-231° C.

Analyses for C₂₆H₂₂N₂O₅:

Calculated: C, 70.58; H, 5.01; N, 6.33

Found: C, 73.12; H, 5.37; N, 5.81.

### EXAMPLE 4

Preparation of [[3-(2-Amino-1,2-dioxoethyl)-1-([1,1′-biphenyl]-3-ylmethyl)-2-methyl-1H-indol-4-yl]oxy]acetic acid, a compound represented by the formula:

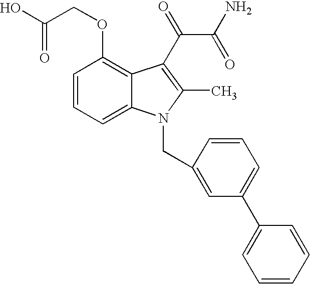

Part A. Preparation of 1-([1,1′-Biphenyl]-3-ylmethyl)-4-methoxy-2-methyl-1H-indole.

Using the method in Example 1, Part C, 805 mg (5 mmol) of 4-methoxy-2-methyl-1H-indole was reacted with 200 mg (5 mmol) of 60% NaH/mineral and then 1.0 g (5 mmol) of 3-(chloromethyl)biphenyl in DMF to give after chromatography on silica gel (eluted with 20% EtOAc/hexane) 1.25 g (76% yield) of 1-([1,1′-biphenyl]-3-ylmethyl)-4-methoxy-2-methyl-1H-indole, mp, 127-131° C.

Analyses for C₂₃H₂₁NO:

Calculated: C, 84.37; H, 6.46; N, 4.27

Found: C, 83.30; H, 6.55; N, 4.07.

Part B. Preparation of 1-([1,1′-Biphenyl]-3-ylmethyl)-4-hydroxy-2-methyl-1H-indole.

By the method used in Example 1, Part D, 1.25 g (3.8 mmol) of 1-([1,1′-biphenyl]-3-ylmethyl)-4-methoxy-2-methyl-1H-indole was O-demethylated by treating it with 15.2 mL of 1M BBr₃/CH₂Cl₂ to give 1.03 g (87% yield) of crude 1-([1,1′-biphenyl]-3-ylmethyl)-4-hydroxy-2-methyl-1H-indole.

Part C. Preparation of [[1-([1,1′-Biphenyl]-3-ylmethyl)-2-methyl-1H-indol-4-yl]oxy]aceticacid methyl ester.

1-([1,1′-Biphenyl]-3-ylmethyl)-4-hydroxy-2-methyl-1H-indole-(1.03 g, 3.3 mmol) was alkylated by treating with 0.31 mL (3.3 mmol) of methyl bromoacetate and 132 mg (3.3 mmol) of 60% NaH/mineral oil in DMF as described in Example 1, Part E. The product was purified by chromatography over silica gel eluting with 20% EtOAc/hexane, to give 1.0 g (79% yield) of [[1-([1,1′-biphenyl]-3-ylmethyl)-2-methyl-1H-indol-4-yl]oxy]acetic acid methyl ester, 99-102° C.

Analyses for C₂₅H₂₃NO₃:

Calculated: C, 77.90; H, 6.01; N, 3.63

Found: C, 77.61; H, 6.09; N, 3.62.

Part D. Preparation of [[3-(2-Amino-1,2-dioxoethyl)-1-([1,1′-biphenyl]-3-ylmethyl)-2-methyl-1H-indol-4-yl]oxy]acetic acid methyl ester.

Oxalyl chloride (0.23 mL, 2.6 mmol) was added to 1.0 g (2.6 mmol) of [[1-([1,1-biphenyl]-3-ylmethyl)-2-methyl-1H-indol-4-yl]oxy]acetic acid methyl ester in 15 mL of methylene chloride and the mixture stirred for 1.3 hours. The mixture was concentrated at reduced pressure, the residue redissolved in 15 mL of methylene chloride and ammonia gas bubbled in for 0.25 hours, stirred for 0.25 hours and concentrated. The residue was stirred with EtOAc/water and the undissolved material filtered to give 300 mg of [[3-(2-amino-1,2-dioxoethyl)-1-([1,1′-biphenyl]-3-ylmethyl)-2-methyl-1H-indol-4-yl]oxy]acetic acid methylester. The EtOAc layer from the filtrate was separated, washed with brine, dried (MgSO₄) and concentrated. The residue was chromatographed on silica gel eluting with EtOAc to give an additional 671 mg of [[3-(2-amino-1,2-dioxoethyl)1-([1,1′-biphenyl]-3-ylmethyl)-2-methyl-1H-indol-4-yl]oxy]acetic acid methyl ester, mp, 175-179° C. The total combined yield of product was 82%.

Analyses for C₂₇H₂₄N₂O₅:

Calculated: C, 71.04; H, 5.30; N, 6.14

Found: C, 71.30; H, 5.41; N, 6.35.

Part E. Preparation of [[3-(2-Amino-1,2-dioxoethyl)-1-([1,1-biphenyl]-3-ylmethyl)-2-methyl-1H-indol-4-yl]oxy]acetic acid.

Using the procedure described in Example 2, Part E, 956 mg (2.1 mmol) of [[3-(2-amino-1,2-dioxoethyl)-1-([1,1′biphenyl]-3-ylmethyl)-2-methyl-1H-indol-4-yl]oxy]acetic acid methyl ester was hydrolyzed in 10 mL of 1N NaOH and 20 mL of MeOH to give 403 mg (41% yield) of [[3-(2-amino-1,2-dioxoethyl)-1-([1,1′-biphenyl]-3-ylmethyl)-2-methyl-1H-indol-4-yl]oxy]acetic acid, sodium salt, mp, >265° C.

Analyses for C₂₆H₂₁N₂O₅Na:

Calculated: C, 67.24; H, 4.56; N, 6.03

Found: C, 67.20; H, 4.58; N, 6.03.

There was also obtained 346 mg (37% yield) of [[3-(2-amino-1,2-dioxoethyl)-1-([1,1′-biphenyl]-3-ylmethyl)-2-methyl-1H-indol-4-yl]oxy]acetic acid, mp, 236-238° C.

Analyses for C₂₆H₂₂N₂O₅:

Calculated: C, 70.58; H, 5.01; N, 6.33

Found: C, 70.58; H, 5.25; N, 6.11.

### EXAMPLE 5

Preparation of 9-benzyl-6-methoxy-1,2,3,4-tetrahydrocarbazole-4-carboxamide.

Part A. Preparation of 9-benzyl-6-methoxy-1,2,3,4-tetrahydrocarbazole-4-carboxylic acid hydrazide.

Compound (5) (R²=6-MeO, R^{3(a)}=H, R⁴=phenyl) was prepared using the method given in Julia, M. and Lenzi, J. Bull. Soc. Chim. France, 1962, 2262-2263. Equimolar quantities of N-benzyl-para-anisidine (3) and ethyl 3-bromo-2-cyclohexanonecarboxylate (Sheehan, J. and Mumaw, C. E. J. Am. Chem. Soc., 1950, 72, 2127-2129) were dissolved in dimethylformamide and stirred in the presence of excess sodium bicarbonate for five days to give ethyl 3-N-benzyl-4-methoxyanilino-2-cyclohexanonecarboxylate (4) which was treated with zinc chloride in refluxing benzene to give (5) (R²=6-MeO R³═H).

A solution of 0.5 gm compound (5) and 2-3 ml of hydrazine hydrate in 30 ml of ethanol was refluxed for 66 hours, cooled, and filtered to give sub-titled compound, 405 mg, 80%, mp 185-187° C.

Elemental Analyses for C₂₁H₂₃N₃02:

Calculated: C 72.18; H 6.63; N 12.02

Found: C 71.90; H 6.68; N 11.87.

Part B. Preparation of 9-benzyl-6-methoxy-1,2,3,4-tetrahydrocarbazole-4-carboxamide.

A mixture of 0.3 gm of the compound of part A above, 2-3 gm of Raney nickel catalyst, and 100 ml of ethanol was refluxed for 2.5 hours, cooled, and the solution was decanted and evaporated in vacuo. The residue was chromatographed on silica gel eluting with a gradient methylene chloride/1-3% methanol to give titled compound, 220 mg, 80%, mp 154-156° C./diethyl ether.

Elemental Analyses for C₂₁H₂₂N₂O₂:

Calculated: C 75.42; H 6.63; N 8.38

Found: C 75.58; H 6.62; N 8.24.

### EXAMPLE 6

Preparation of 4-[(9-benzyl-4-carbamoyl-1,2,3,4-tetrahydrocarbazol-6-yl)oxy]butyric acid

A solution of 280 mg of compound (7) (R²=6-MeO, R³═H, R⁴=phenyl) in 25 ml of dichloromethane was treated with 3 ml of 1M boron tribromide in dichloromethane for 2.75 hours, washed with water, washed with brine, dried over sodium sulfate, and evaporated in vacuo to give 290 mg of crude compound (7) (R²=6-OH, R³=H) which was dissolved in 10 ml of tetrahydrofuran and 50 ml of dimethylsulfoxide and treated with 40 mg of sodium hydride (60% in mineral oil) for 10 minutes and then with 0.15 ml of ethyl 4-bromobutyrate for 1.75 hours. The solution was diluted with ethyl acetate and water and the organic phase washed with water, washed with brine, dried over sodium sulfate, and evaporated in vacuo. The residue was chromatographed on silica gel eluting with a gradient methylene chloride/0-2% methanol to give 340 mg of compound (9) (R²=-O(CH₂)₄CO₂Et, R³=H) which was dissolved in 25 ml of ethanol containing 2-3 ml of 2N sodium hydroxide and stirred 4.25 hours, acidified with hydrochloric acid and extracted with ethyl acetate. The organic phase was washed with brine, dried over sodium sulfate, and concentrated in vacuo to give title compound, 300 mg, 90%, mp 199-200° C.

Elemental Analyses for C₂₄H₂₆N₂O₄:

Calculated: C 70.92; H 6.45; N 6.89

Found: C 70.63; H 6.49; N 6.87

### EXAMPLE 7

Preparation of 3-[(9-benzyl-4-carbamoyl-1,2,3,4-tetrahydrocarbazol-6-yl)oxy]propylphosphonic acid

Part A. Preparation of dimethyl-3-[(9-benzyl-4-carbamoyl-1,2,3,4-tetrahydrocarbazol-6-yl)oxy]propylphosphonic acid.

Part A. A solution of 840 mg of compound (7) (R^{2(a)}=OH R³=H) (prepared as in example 2) in 20 ml of tetrahydrofuran and 70 ml of dimethylsulfoxide was treated with 120 mg of sodium hydride (60% in mineral oil) for 10 minutes and then with 700 mg of dimethyl 3-bromopropylphosphonate for 5 hours. The solution was diluted with water and ethyl acetate. The organic phase was washed with water, washed with brine, dried over sodium sulfate, and evaporated in vacuo to give sub-titled compound, 940 mg, 75%, amorphous solid.

Elemental Analyses for C₂₅H₃₁N₂O₅P:

Calculated: C 63.82; H 6.64; N 5.95

Found: C 63.94; H 6.58; N 6.15

Part B. A solution of 450 mg of the compound of Part A and 1.5 ml of trimethylsilyl bromide in 25 ml of dichloromethane was stirred for 16 hours and then evaporated in vacuo. The residue was dissolved in 25 ml of methanol, stirred for 2.5 hours, evaporated in vacuo, and crystallized from a mixture of ethyl acetate and methanol to give title compound, 325 mg, 78%, mp 200-201° C.

Elemental Analyses for C₂₃H₂₇N₂O₅P.2H₂O:

Calculated: C 57.73; H 6.53; N 5.86

Found: C 57.51; H 5.94; N 6.00

### EXAMPLE 8

Preparation of 2-[(9-benzyl-4-carbamoyl-1,2,3,4-tetrahydro-carbazol-6-yl)oxy]methylbenzoic acid

Part A. Preparation of methyl 2-[(9-benzyl-4-carbamoyl-1,2,3,4-tetrahydrocarbazol-6-yl)oxy]methylbenzoate.

A solution of 700 mg of compound (7) (R^{2(a)}=OH, R³=H, R⁴=phenyl) (prepared as in example 2) in 20 ml of tetrahydrofuran and 70 ml of dimethylsulfoxide was treated with 100 mg of sodium hydride (60% in mineral oil) for 10 minutes and then with 575 mg of methyl 2-bromomethylbenzoate for 2.5 hours. The solution was diluted with water and ethyl acetate. The organic phase was washed with water, washed with brine, and evaporated in vacuo. The residue was chromatographed on silica gel eluting with a gradient methylene chloride/0-2% methanol to give sub-titled compound, 840 mg, 90%, mp 119-120° C./Et₂O.

Elemental Analyses for C₂₉H₂₈N₂O₄:

Calculated: C 74.34; H 6.02; N 5.98

Found: C 74.22; H 6.03; N 5.70

Part B. Preparation of 2-[(9-benzyl-4-carbamoyl-1,2,3,4-tetrahydrocarbazol-6-yl)oxy]methylbenzoic acid.

A solution of 670 mg of the compound of Part A and 5 ml of 2N sodium hydroxide in 100 ml of ethanol and 15 ml of tetrahydrofuran was stirred for 16.5 hours, acidified with hydrochloric acid, and extracted with ethyl acetate. The organic phase was washed with brine, dried over sodium sulfate, and evaporated in vacuo. The residue was chromatographed on silica gel eluting with a gradient methylene chloride/2-4% methanol to give title compound, 230 mg, 34%, amorphous solid.

Elemental Analyses for C₂₈H₂₆N₂O₄—H₂O:

Calculated: C 71.17; H 5.97; N 5.93

Found: C 71.31; H 5.68; N 5.65

### EXAMPLE 9

Preparation of 9-benzyl-5,7-dimethoxy-1,2,3,4-tetrahydrocarbazole-4-carboxylic acid hydrazide

Part A. Preparation of 9-benzyl-3,5-dimethoxyaniline.

A solution of 25 gm. (0.163 mol) of 3,5-dimethoxyaniline and 18.3 ml. (0.18 mol) of benzaldehyde in 300 ml. of methanol was cooled in ice-water and treated with 10.3 gm. (0.18 mol) of sodium cyanoborohydride in portions. The solution was stirred and cooled for 3 hours, treated with 1-2 gm. of sodium borohydride for 30 minutes, diluted with water and extracted with ethyl acetate. The organic phase was washed with brine, dried over sodium sulfate, and evaporated in vacuo. The residue was chromatographed on silica gel eluting with a gradient hexane/15-70% ether to give 9-benzyl-3,5-dimethoxyaniline, 28.0 gm., 71%, as an oil.

Elemental Analyses for C₁₅H₁₇NO₂:

Calculated: C 74.05; H 7.04; N 5.76

Found: C 74.30; H 7.12; N 5.70

Part B. Preparation of 9-benzyl-5,7-dimethoxy-1,2,3,4-tetrahydrocarbazole-4-carboxylic acid hydrazide.

A solution of 9.72 gm of the compound of part A and 4.98 gm of 2-carbethoxy-6-bromocyclohexanone (J. Sheehan and C. E. Mumaw, J.Am.Chem.Soc., 72, 2127-2129, (1950).) in 125 ml of benzene was refluxed for 72 hours, cooled, filtered, and evaporated in vacuo. The residue (12 gm) and 10 gm of zinc chloride were refluxed in 250 ml of benzene for 6 hours, cooled and evaporated in vacuo. The residue was taken up in ethyl acetate, washed with 1N hydrochloric acid, washed with water, dried over sodium sulfate, and evaporated in vacuo. The residue was chromatographed on silica gel eluting with a gradient toluene/0-5% ethyl acetate to give compound (5) (R²=5-MeO R³=7-MeO R⁴=phenyl), 1.88 gm which was dissolved in 100 ml of ethanol containing 10 ml of hydrazine hydrate and refluxed for 5 days, cooled, the solution decanted, diluted with ethyl acetate, washed with brine, dried over sodium sulfate, and evaporated in vacuo to give title compound, 1.1 gm, 60%, mp 189-190° C./CH₂Cl₂-EtOH.

Elemental Analyses for C₂₂H₂₅N₃O₃:

Calculated: C 69.64; H 6.64; N 11.07

Found: C 69.59; H 6.74; N 10.84

### EXAMPLE 9

Preparation of 9-benzyl-5,7-dimethoxy-1,2,3,4-tetrahydrocarbazole-4-carboxamide

A mixture of 980 mg of the compound of example 5, 2 gm of Raney nickel catalyst, 1-2 ml of hydrazine hydrate, and 125 ml of ethanol was refluxed 1 hour, the solution decanted, diluted with ethyl acetate, washed with water, washed with brine, dried over sodium sulfate, and evaporated in vacuo. The residue was chromatographed on silica gel eluting with a gradient methylene chloride/1-3% methanol to give title compound, 820 mg, 84%, mp 190192° C./EtOH.

Elemental Analyses for C₂₂H₂₄N₂O₃:

Calculated: C 72.51; H 6.64; N 7.69

Found: C 71.88; H 6.89; N 7.81

### EXAMPLE 10

Preparation of [9-benzyl-4-carbamoyl-7-methoxy-1,2,3,4-tetrahydrocarbazol-5-yl]oxyacetic acid

Part A. Preparation of 9-benzyl-5-hydroxy-7-methoxy-1,2,3,4-tetrahydrocarbazole-4-carboxamide.

A solution of 1.75 gm. (4.8 mmol) of the compound of example 6 in 50 ml. of dimethylformamide was mixed with a solution of sodium thioethoxide (13.5 mmol) in 75 ml. of dimethylformamide and then heated at 100° C. for 21 hours. The mixture was cooled, diluted with water, acidified with hydrochloric acid, and extracted with ethyl acetate. The organic phase was washed with brine, dried over sodium sulfate, and evaporated in vacuo. The residue was chromatographed on silica gel eluting with a gradient methylene chloride/0-4% methanol to give the sub-titled product, 825 mg., 50%, mp 225-7° C./ethanol.

Elemental Analyses for C₂₁H₂₂N₂O₃:

Calculated: C 71.98; H 6.33; N 7.99

Found: C 71.71; H 6.37; N 7.72

Part B. Preparation of [9-benzyl-4-carbamoyl-7-methoxy-1,2,3,4-tetrahydrocarbazol-5-yl]oxyacetic acid ethyl ester.

A solution of 700 mg. (2.0 mmol) of the product from Part A in 70 ml. of dimethylformamide and 15 ml. of tetrahydrofuran was treated with 100 mg. of sodium hydride (60% in mineral oil; 2.5 mmol) for 10 minutes and then with 0.3 ml. (2.7 mmol) of ethyl bromoacetate for 3 hours. The mixture was diluted with ethyl acetate, washed with water, washed with brine, dried over sodium sulfate, and evaporated in vacuo. The residue was chromatographed on silica gel eluting with a gradient methylene chloride/1-2% methanol to give sub-titled product, 670 mg., 77%, mp 167169° C./ether.

Elemental Analyses for C₂₅H₂₈N₂O₅:

Calculated: C 68.79; H 6.47; N 6.42

Found: C 69.57; H 6.39; N 5.77

Part C. Preparation of [9-benzyl-4-carbamoyl-7-methoxy-1,2,3,4-tetrahydrocarbazol-5-yl]oxyacetic acid.

A suspension of 650 mg. of the product from Part B in 20 ml. of tetrahydrofuran and 70 ml. of ethanol was treated with 5 ml. of 2N sodium hydroxide and the resulting solution was stirred for 15.5 hours. The solution was diluted with ethyl acetate and water and acidified with hydrochloric acid. The organic phase was washed with brine, dried over sodium sulfate, concentrated in vacuo, and filtered to give title product, 540 mg., 87%, mp 251254° C.

Elemental Analyses for C₂₃H₂₄N₂O₅:

Calculated: C 67.63; H 5.92; N 6.86

Found: C 67.73; H 5.74; N 6.82

### EXAMPLE 11

Preparation of 3-[(9-benzyl-4-carbamoyl-7-n-octyl-1,2,3,4-tetrahydrocarbazol-6-yl)oxy]propylphosphonic acid

Part A. Preparation of ethyl 9-benzyl-6-methoxy-7-n-octyl-1,2,3,4-tetrahydrocarbazole-4-carboxylate.

A mixture of 1.3 gm of 9-benzyl-4-methoxy-5-n-octylaniline (3) (prepared by the procedures of Example 9, Part A, utilizing n-heptyltriphenylphosphonium bromide), 1.0 gm of 2-carbethoxy-6-bromocyclohexanone, 675 mg of sodium bicarbonate, and 40 ml of dimethylformamide was stirred for 5 days, diluted with ethyl acetate, washed with water, washed with brine, dried over sodium sulfate, and evaporated in vacuo. The residue and 10 gm of zinc chloride in 250 ml of benzene was refluxed for 6 hours, cooled, diluted with ethyl acetate, washed with 1N hydrochloric acid, washed with brine dried over sodium sulfate, and evaporated in vacuo. The residue was chromatographed on silica gel eluting with a gradient hexane/10-15% diethyl ether to give compound (5) (R²=6-MeO R^{3a}=7-n-octyl R⁴=phenyl), 930 mg, which was dissolved in 30 ml of benzene and treated with 15 ml of an 0.67M solution of methyl chloroaluminum amide at 50° C. for 16 hours, cooled, decomposed with ice and 1N hydrochloric acid, and extracted with ethyl acetate. The organic phase was washed with brine, dried over sodium sulfate, and evaporated in vacuo to give sub-titled compound, 795 mg, 91%, mp 131-133° C./Et₂O.

Elemental Analyses for C₂₉H₃₈N₂O₂:

Calculated: C 77.97; H 8.58; N 6.27

Found: C 77.75; H 8.62; N 5.99

Part B. Preparation of 9-benzyl-6-hydroxy-7-n-octyl-1,2,3,4-tetrahydrocarbazole-4-carboxamide.

A solution of 770 mg of the compound of Part A, in 75 ml of dichloromethane was treated with 10 ml of 1M boron tribromide in dichloromethane for 1.75 hours and then decomposed with ice and 1N hydrochloric acid. The organic phase was washed with brine, dried over sodium sulfate, and evaporated in vacuo. The residue was chromatographed on silica gel eluting with a gradient methylene chloride/1-3% methanol to give sub-titled compound, 360 mg, 47%, mp 223-225° C.

Elemental Analyses for C₂₈H₃₆N₂O₂:

Calculated: C 77.74; H 8.39; N 6.48

Found: C 77.97; H 8.45; N 6.40

Part C. Preparation of 3-[(9-benzyl-4-carbamoyl-7-n-octyl-1,2,3,4-tetrahydrocarbazol-6-yl)oxy]propylphosphonic acid.

A solution of 360 mg of the compound of Part B in 10 ml of tetrahydrofuran and 70 ml of dimethylformamide was treated with 40 mg of sodium hydride (60% in mineral oil) for 15 minutes and then with 230 mg of dimethyl 3-bromopropylphosphonate for 4 hours, diluted with water, and extracted with ethyl acetate. The organic phase was washed with brine, dried over sodium sulfate, and evaporated in vacuo. The residue was chromatographed on silica gel eluting with a gradient methylene chloride/1-5% methanol to give compound (9) (R²=6-(MeO)₂P═O(CH₂)₃, R³=7-n-octyl, R⁴=phenyl), 290 mg, which was dissolved in 30 ml of dichloromethane and 1 ml of trimethylsilyl bromide, stirred for 20 hours, evaporated in vacuo, dissolved in 30 ml of methanol, stirred 2.25 hours, evaporated in vacuo, and crystallized from a mixture of benzene-methanol-diethyl ether, to give title compound, 185 mg, 67%, mp 160-162° C.

Elemental Analyses for C₃₁H₄₃N₂O₅P.2H₂O:

Calculated: C 63.03; H 8.02; N 4.74

Found: C 63.18; H 7.53; N 4.93

### EXAMPLE 12

Preparation of 4-[(9-benzyl-4-carbamoyl-7-ethyl-1,2,3,4-tetrahydrocabazol-6-yl)oxy]butyric acid

Part A. Preparation of benzyl 9-benzyl-6-methoxy-7-ethyl-1,2,3,4-tetrahydrocarbazole-4-carboxylate.

A suspension of 22 gm (0.13 mol) of 5-nitrosalicaldehyde, 10 ml (0.16 mol) of methyl iodide, 28 gm (0.2 mol) of potassium carbonate, 75 ml of dimethylsulfoxide, and 125 ml of 2-butanone was refluxed for 20 hours, cooled, diluted with ethyl acetate, washed with water, washed with brine, dried over sodium sulfate, and evaporated in vacuo. The residue was chromatographed on silica gel eluting with a gradient hexane/10-50% ether to give (1) (R²=4-MeO R^{3(a)}=3-CHO), 15 gm, 67%.

A suspension of 19.6 gm (0.055 mol) of methyltriphenylphosphonium bromide in 300 ml of tetrahydrofuran was cooled at −5° C. and treated slowly with 35 ml of n-butyl lithium (1.6M in hexane, 0.055 mol). The cooling bath was removed and the mixture allowed to warm to room temperature over 45 minutes. A solution of 9.1 gm (0.050 mol) of the aldehyde prepared above was slowly added to this yellow solution and stirred 2 hours, diluted with water, and extracted with ethyl acetate. The organic phase was washed with brine, dried over sodium sulfate, and evaporated in vacuo. The residue was chromatographed on silica gel eluting with a gradient hexane/15-50% ether to give (1) (R²=4-MeO R^{3(a)}=3-CH₂═CH), 7.7 gm, 86%.

This product and 3 gm of 10% Pd/C in 200 ml of ethanol was stirred under 1 atmosphere of hydrogen for 6 hours, filtered, and evaporated in vacuo to give (2) (R²=4-MeO R^{3(a)}=3-CH₃CH₂). This crude product and 5 ml of benzaldehyde in 150 ml of methanol was stirred at 0-5° C., while adding in portions 2.5 gm of sodium cyanoborohydride. After an additional 60 minutes at this temperature the mixture was diluted with water and extracted with ethyl acetate. The organic phase was washed with brine, dried over sodium sulfate, and evaporated in vacuo. The residue was chromatographed on silica gel eluting with a gradient hexane/5-15% ether to give 9-benzyl-4-methoxy-5-ethylaniline, 6.0 gm, 60%.

A mixture of 3.3 gm of 9-benzyl-4-methoxy-5-ethylaniline, 3.7 gm of 2-carbethoxy-6-bromocyclohexanone, 1.3 gm of sodium bicarbonate, and 100 ml of dimethylformamide was stirred for 5 days, diluted with ethyl acetate, washed with water, washed with brine, dried over sodium sulfate, and evaporated in vacuo. The residue and 10 gm of zinc chloride in 250 ml of benzene was refluxed for 1.75 hours, cooled, diluted with ethyl acetate, washed with 1N hydrochloric acid, washed with brine, dried over sodium sulfate, and evaporated in vacuo. The residue was chromatographed on silica gel eluting with a gradient hexane/10-20% diethyl ether to give sub-titled compound, 2.6 gm, 50%, mp 85-86/EtOH.

Elemental Analyses for C₂₅H₂₉NO₃:

Calculated: C 76.70; H 7.47; N 3.58

Found: C 77.00; H 7.56; N 3.69

Part B. Preparation of 9-benzyl-6-methoxy-7-ethyl-1,2,3,4-tetrahydrocarbazole-4 carboxamide.

A solution of 2.6 gm of the compound of Part A in 75 ml of benzene and 30 ml of 0.67M methyl chloroaluminum amide in benzene/toluene was heated at 50° C. for 24 hours, cooled, decomposed with 1N hydrochloric acid, and extracted with ethyl acetate. The organic phase was washed with brine, dried over sodium sulfate, and evaporated in vacuo to give sub-titled compound, 2.2 gm, 91%, mp 168-169° C./CH₂Cl₂-EtOH.

Elemental Analyses for C₂₃H₂₆N₂O₂:

Calculated: C 76.21; H 7.23; N 7.73

Found: C 76.55; H 7.74; N 6.84

Part C. Preparation of 4-[(9-benzyl-4-carbamoyl-7-ethyl-1,2,3,4-tetrahydrocarbazol-6-yl)oxy]butyric acid.

A solution of 2.1 gm of the compound of Part B in 150 ml of dichloromethane and 15 ml of 1M boron tribromide in dichloromethane was stirred for 2 hours, decomposed with ice and water, and the organic phase washed with brine, dried over sodium sulfate, and evaporated in vacuo to give compound (7) (R^{2(a)}=6-HO, R³=7-ethyl, R⁴=phenyl), 1.6 gm, 80%, mp 255° C. dec./methylene chloride-ethanol. A solution of 750 mg of this material in 20 ml of tetrahydrofuran and 70 ml of dimethylformamide was treated with 100 mg of sodium hydride (60% in mineral oil) for 10 minutes and then with 0.33 ml of ethyl 4-bromobutyrate for 4.5 hours, diluted with ethyl acetate, washed with water, washed with brine, dried over sodium sulfate, and evaporated in vacuo. The residue was chromatographed on silica gel eluting with a gradient methylene chloride/1-3% methanol to give compound (9) (R²=6-EtO₂CCH2CH₂CH₂O, R³=7-ethyl), 625 mg, 64%, mp 134-136/ethylene chloride-ethanol, which was dissolved in 10 ml of tetrahydrofuran and 40 ml of ethanol containing 3 ml of 2N sodium hydroxide and stirred for 22 hours. The solution was acidified with hydrochloric acid and extracted with ethyl acetate. The organic phase was washed with brine, dried over sodium sulfate, and evaporated in vacuo to give title compound, 410 mg, 73%, mp 208-210° C./CH₂Cl₂-EtOH.

Elemental Analyses for C₂₆H₃₀N₂O₄.0.4H₂O:

Calculated: C 64.05; H 7.15; N 5.54

Found: C 71.76; H 6.90; N 6.56

### EXAMPLE 13

Preparation of 3-[(9-benzyl-4-carbamoyl-7-ethyl-1,2,3,4-tetrahydrocarbazol-6-yl)oxy]propylphosphonic acid

Part A. Preparation of dimethyl 3-[(9-benzyl-4-carbamoyl-7-ethyl-1,2,3,4-tetrahydrocarbazol-6-yl)oxy]propylphosphonate.

A solution of 750 mg of the compound of example 8 in 20 ml of tetrahydrofuran and 75 ml of dimethylformamide was treated with 100 mg of sodium hydride (60% in mineral oil) for 10 minutes and then with 510 mg of dimethyl 3-bromopropylphosphonate for 5.25 hours, diluted with ethyl acetate, washed with water, washed with brine, dried over sodium sulfate, and evaporated in vacuo. The residue was chromatographed on silica gel eluting with a gradient methylene chloride/1-5% methanol to give sub-titled, 655 mg, 61%, amorphous solid.

Elemental Analyses for C₂₇H₃₅N₂O₅P:

Calculated: C 64.12; H 7.15; N 5.54

Found: C 64.27; H 7.00; N 5.92

Part B. Preparation of 3-[(9-benzyl-4-carbamoyl-7-ethyl-1,2,3,4-tetrahydrocarbazol-6-yl)oxy]propylphosphonic acid.

A solution of the compound of Part A and 1 ml of trimethylsilyl bromide in 30 ml of dichloromethane was stirred 18 hours, evaporated in vacuo, dissolved in 25 ml of methanol, stirred 3.75 hours, and concentrated in vacuo to give title compound, 475 mg, 80%, mp 235-238° C. dec.

Elemental Analyses for C₂₅H₃₁N₂O₅P:

Calculated: C 63.82; H 6.64; N 5.95

Found: C 63.56; H 6.62; N 6.07

### The Practice of the Method of the Invention

The practice of this invention involves initiating administration of a sPLA₂ inhibitor compound of formula I or II, or combination thereof, or a combination therapy of a sPLA₂ inhibitor compound of formula I or II and an effective therapeutic drug, compound or treatment method to a patient susceptible to sepsis or in need thereof, that maximally enhances the treatment and/or prevention ability of the active ingredient(s). This invention is based on the discovery that selected sPLA₂ inhibitors are more effective in preventing and treating sepsis when administered within 24 hours of the patient displaying abnormally high sPLA₂ levels. Particularly preferred is administration prior to the onset of sepsis or within 24 hours after the first organ failure and continuing for about 1 to 7 days thereafter or as determined by a treating physician. More particularly preferred is initiation of administration of sPLA₂ inhibitor compound, racemate, pharmaceutically acceptable salt, solvate, tautomer or prodrug derivative thereof, within 18 hours after first organ failure or observation of elevated sPLA₂ levels. Even more particularly preferred is initiation of administration of sPLA₂ inhibitor compound, racemate, pharmaceutically acceptable salt, solvate, tautomer or prodrug derivative thereof, within 12 hours after first organ failure or observation of elevated sPLA₂ levels. Most particularly preferred is initiation of administration of sPLA₂ inhibitor compound, racemate, pharmaceutically acceptable salt, solvate, tautomer or prodrug derivative thereof, within 6 hours after first organ failure or observation of elevated sPLA₂ levels.

The method of the invention can be practiced using pharmaceutical formulations containing sPLA₂ inhibitors of formula I or II racemate, pharmaceutically acceptable salt, solvate, tautomer or prodrug derivative thereof (preferably, sPLA₂ inhibitors identified as preferred herein) or formulations containing such sPLA₂ inhibitors as taught in the preceding section in combination with other approved effective therapies including drugs for the treatment and/or prevention of sepsis. Other effective therapies useful as combination therapy or co-agent for the method of the invention include activated protein C, preferably recombinant human activated protein C, and N-[o-(p-pivaloyloxybenzene)sulfonylaminobenzoyl]glycine also known as N-[2-[4-(2,2-dimethylpropionyloxy)phenylsulfonylamino]benzoyl]aminoacetic acid. Although it is believed that the underlying causes of sepsis will not be prevented by the method of this invention, the symptoms will be reduced in severity or extent by administration of sPLA₂ inhibitors (and their formulations), sufficient to prevent the effects of sepsis or death in a significant number of patients when given according to the method of this invention.

The dosage administered will vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active compound can be about 0.1 to 500 milligrams per kilogram of body weight. Ordinarily 0.5 to 50, and preferably 1 to 25 milligrams per kilogram per day given in divided doses 1 to 6 times a day or in sustained release form is effective to obtain desired results. Depending on the patient's state, a lower dose range is given prior to the onset of sepsis or sepsis inducing conditions (i.e., as determined by increase in sPLA₂ levels), while a higher dose may be given when aggressive intervention is indicated to combat rising sPLA₂ levels.

In general, the sPLA₂ inhibitor will be administered to an animal so that a therapeutically effective amount is received. A therapeutically effective amount may conventionally be determined for an individual patient by administering the active compound in increasing doses and observing the effect on the patient, for example, reduction in fever, suppression of increasing sPLA₂ activity levels, or a reduction in other symptoms associated with sepsis.

Generally, the compound must be administered in a manner and a dose to achieve in the animal a blood level concentration of sPLA₂ inhibitor of from 10 to 5000 nanograms/ml and preferably a concentration of 100 to 1000 nanograms/ml.

The treatment regimen may stretch over many days to months or to years as determined by a competent caregiver (treating physician). Oral dosing and/or intravenous infusion are preferred for patient convenience and tolerance. With oral dosing, one to four oral doses per day, each from about 0.01 to 25 mg/kg of body weight with preferred doses being from about 0.1 mg/kg to about 5 mg/kg.

Proportion and Weight of Active Compounds Used in the Method of the Invention

The 1H-indole-3-glyoxylamide compound may be used at a concentration of 0.1 to 99.9 weight percent of the formulation.

Preferably the pharmaceutical formulation is in unit dosage form. The unit dosage form can be a capsule or tablet itself, or the appropriate number of any of these. The quantity of active compound in a unit dose of composition may be varied or adjusted from about 0.1 to about 1000 milligrams or more according to the particular treatment involved.

Compositions (dosage forms) suitable for internal administration contain from about 1 milligram to about 500 milligrams of active compound per unit. In these pharmaceutical compositions the active compound will ordinarily be present in an amount of about 0.5-95% by weight based on the total weight of the composition.

Examples of useful pharmaceutical compositions and their proportions of ingredients are illustrated as follows:

Capsules: Capsules may be prepared by filling standard two-piece hard gelatin capsules each with 50 mg of powdered active compound, 175 mg of lactose, 24 mg of talc, and 6 mg of magnesium stearate.

Soft Gelatin Capsules: A mixture of active compound in soybean oil is prepared and injected by means of a positive displacement pump into gelatin to form soft gelatin capsules containing 50 mg of the active compound. The capsules are washed in petroleum ether and dried.

Tablets: Tablets may be prepared by conventional procedures so that the dosage unit is 50 mg of active compound, 6 mg of magnesium stearate, 70 mg of microcrystalline cellulose, 11 mg of cornstarch, and 225 mg of lactose. Appropriate coatings may be applied to increase palatability or delay absorption.

Suspensions: An aqueous suspension is prepared for oral administration so that each 5 ml contain 25 mg of finely divided active compound, 200 mg of sodium carboxymethyl cellulose, 5 mg of sodium benzoate, 1.0 g of sorbitol solution, U.S.P., and 0.025 mg of vanillin.

Injectables: A parenteral composition suitable for administration by injection is prepared by stirring 1.5% by weight of active compound in 10% by volume propylene glycol and water. The solution is sterilized by commonly used techniques.

Nasal Spray: An aqueous solution in prepared such that each 1 ml contains 10 mg of active compound, 1.8 mg methylparaben, 0.2 mg propylparaben and 10 mg methylcellulose. The solution in dispensed into 1 ml vials.

The active compound may be used at a concentration of 0.01 to 99.9 weight percent of the formulation.

Aerosol formulations are capable of dispersing into particle sizes of from about 0.5 to about 10 microns and have sufficient sPLA₂ inhibitor to achieve concentrations of the inhibitor on the airway surfaces of from about 10⁻¹⁰ to 10⁻² moles per liter.

Parenteral Administration (particularly, intravenous administration) is often preferred in instances where rapid alleviation of patient distress is required such an when analysis shown an increase in sPLA₂ levels or up to 24 hours after the first organ failure. With parenteral administration doses of 0.01 to 200 mg/kg/day administered continuously or intermittently throughout the day may be used. For parenteral administration, the compound may be administered in a physiologic saline vehicle (e.g., 0.9% normal saline, 0.45% normal saline, etc.) a dextrose vehicle (e.g., 5% dextrose in water), or a combination of saline and dextrose vehicle (0.9% normal saline in 5% dextrose).

Parenteral administration is also preferably effected by use of a freeze dried lyophilized composition(s) of a compound useful for the practice of the present invention i.e. a compound of formula I.

Identity and Proportion of Ingredients in the Lyophilized Compositions Useful for the Practice of the of the Invention:

In one embodiment, the practice of the present invention involves use of a pharmaceutical composition which comprises a sPLA₂ inhibitor i.e., a Compound of formula (Vb) as Active Ingredient and an effective amount of a Solubilizer acting as a chelating agent, for example, preferably at least one compound selected from citric acid, edetic acid (e.g., EDTA, disodium), polyphosphoric acid and their salts, more preferably sodium citrate. Examples of polyphosphoric acid and their salts are potassium polyphosphate as described in the Japanese standards of food additives, 6-th ed., and sodium polyphosphate as described in the Japanese standards of food additives, 6-th ed., or the Japanese standards of cosmetic ingredients, 2-nd ed. Sodium citrate is available as trisodium citrate anhydrous, trisodium citrate dihydrate, and trisodium citrate pentahydrate, but is most conveniently and preferably used in the form of trisodium citrate dyhydrate (mol. wt. 294.10).

The amount of the Solubilizer varies with the kind of the Solubilizer and the concentration of Compound (Vb) for example, and may be from about 1% to about 400% (w/w), preferably 1 to 200% (w/w), most preferably 1 to 100% (w/w) of the amount of the equivalent acid of Compound (Vb). For pharmaceutical compositions using sodium citrate the weight of Solubilizer is from 10% to 60% (w/w) and most preferable 25% to 50% (w/w) of the amount of the equivalent acid of for example, Compound (Vb).

Preferably, the pharmaceutical composition described in the preceding paragraph also has an effective amount of a Stabilizer. The Stabilizer is at least one pharmaceutically acceptable compound selected from solid sugars and sugar-alcohols more preferably at least one compound selected from mannitol, xylitol, sorbitol, glucose, fructose, lactose and maltose. Mannitol is the most preferred Stabilizer ingredient.

The amount of the Stabilizer varies with the kind of Stabilizer and the concentration of sPLA₂ inhibitor i.e., Compound (vb), and may be 40% to 500% (w/w), preferably from 50% to 300% (w/w), more preferably from 50 to 200% (w/w), most preferably from 100% to 200% (w/w) of the amount of the equivalent acid of such as Compound (Vb).

Without departing from the object and scope of the Present invention, other pharmaceutically acceptable additive agents may optionally be added to the preparations useful for the practice of the present invention. Where a solution according to the invention is prepared for injection, and isotonizing agent, a soothing agent or other additives may be added thereto.

Preferably, the pharmaceutical compositions described above are salt-free except for the Active Ingredient, the Solubilizer and the Stabilizer.

Lyophilized Compositions Useful for the Practice of the Invention:

Preferably, the pharmaceutical compositions described in the preceding section are lyophilized. Most preferably the lyophilized composition is prepared with an annealing step by employing the collapse temperature characteristics of Compound useful for the practice of the invention i.e., compound of formula (Vb).

For example, the lyophilized composition contains Solubilizer from about 1 to about 200% (w/w) of the amount of the equivalent acid of Compound (Vb). The proportions of the Solubilizer are the same as those set out in the preceding section for the pharmaceutical composition. When the Solubilizer is disodium EDTA (or its acid or other salts) it is preferably used from about 1% to about 15% (w/w) of the amount of the equivalent acid of for example, the Compound (Vb)

The identity and proportions of Stabilizer are the same as those set out in the preceding section for the pharmaceutical composition. Mannitol is most preferred as the Stabilizer ingredient of the lyophilized compositions of the invention.

Table 1 lists Specific Preferred Lypholyzed Compositions of the Invention (all amounts in milligrams):

TABLE 1

| A.I. | Na Citrate | Mannitol | EDTA |
|---|---|---|---|
| 100 | 50 | 200 | — |
| 100 | 100 | 200 | — |
| 100 | 75 | 200 | — |
| 100 | — | 200 | 1 |
| 100 | — | 200 | 8 |
| 100 | — | 200 | 15 |

A.I. = Active Ingredient,
Na Citrate = trisodium citrate dihydrate
EDTA = ethylenediaminetetraacetic acid, disodium salt

Preferably, the solid lyophilized compositions of the invention are substantially salt-free, except for Compound (Vb) for example, and the Stabilizer and Solubilizer contained therein.

The lyophilized pharmaceutical formulation can be dissolved in a pharmaceutically acceptable carrier, such as sterile water, sterile water optionally containing saline and/or sterile water containing sugars. For example, for intravenous injection the compositions of the invention may be dissolved in at a concentration of 2 mg/ml in a 4% dextrose/0.5% Na citrate aqueous solution.

Method of Making the Lyophilized Compositions Useful for the Practice of the Invention:

The lyophilized compositions useful for the practice of the present invention refer to a preparation prepared by freeze drying a solution containing a sPLA₂ inhibitor compound, i.e., Compound (Vb), optionally being subjected to a heat treating process, and being dried in a high vacuum for sublimating water. Such lyophilized preparations include lyophilized preparations for injection as mentioned above. The lyophilized preparation may be produced by conventional methods including tray lyophilization, spray lyophilization and vial lyophilization methods. Vial lyophilization is advantageous for preparing multi-dosage units of the invention as described, infra.

In order to obtain a solution of Compound (Vb) by the process of the present invention, Compound (Vb), a Solubilizer and a solvent are mixed and stirred until the mixture becomes clear. The solvent is preferably an aqueous solvent such as water, purified water, water for injection, isotonic sodium chloride solution or glucose injection as described in the Japanese Pharmacopoeia, more preferably a salt-free aqueous solvent such as water, purified water, water for injection or glucose solutions for injection.

Alternatively, a suitable solvent for forming a solution from the composition of the invention is any injectable solution as further exemplified by those described in The United States Pharmacopeia (1995, ISBN 0195-7996), for example, “Sterile Water for Injection”, “Dextrose and Sodium Chloride Injection”, “Dextrose Injection”, “Mannitol Injection” or “Mannitol in Sodium Chloride Injection.”

In order to obtain a lyophilized preparation of Compound (Vb) for example, by the process of the present invention, first, a processing solution prior to lyophilization is prepared. The processing solution before lyophilization is a solution prepared by mixing and stirring Compound (Vb), a Solubilizer and a solvent, preferably Compound (Vb), a Solubilizer, a Stabilizer and a solvent, until the mixture becomes clear. For the sequence of addition of the ingredients to the solvent it is highly preferred to first dissolve the Solubilizer and Stabiliser, and thereafter dissolve co (Vb). The solvent is preferably an aqueous solvent such as previously set out above and an described in the Japanese Pharmacopoeia more preferably a salt-free aqueous solvent such as water, purified water, water for injection or glucose injection. The processing solution before lyophilization of Compound (Vb) for example, may contain Compound (Vb) for example, at a concentration of from about 0.5% to 2% (w/w). If desired, the processing solution before lyophilization may be subjected to a filtration process.

The filtration process includes, for example in the case of injection preparations, a sterilizing filtration and/or an ultra filtration of the processing solution before lyophilization to eliminate microorganisms or other contaminating matter from the processing solution before lyophilization.

If desired, the processing solution before lyophilization may be subjected to a distributing process. The distributing process includes, for example in the case of vial lyophilizations, a process distributing a suitable volume of the processing solution before lyophilization into vials taking the concentration of a sPLA₂ inhibitor compound i.e., Compound (Vb) into consideration in order that vial products carry a desired amount of sPLA₂ inhibitor compounds.

A lyophilization process is performed as follows:

Preferably, the lyophilized composition is prepared by a sequential heating and cooling process. A process for preparing a lyophilized composition comprises the sequential steps of:

(a) dissolving lyophilized composition ingredients comprising a sPLA₂ inhibitor e.g. Sodium [[3-(2-amino-1,2-dioxoethyl)-2-ethyl-1-phenylmethyl)-1H-indol-4-yl]oxy]acetate, (compound (Vb)), Soulubilizer, and Stabilizer in an aqueous solvent;

(b) cooling the processing solution of step (a) to a temperature below −33° C.;

(c) heating the product of step (b) to a temperature above −33° C.;

(d) cooling the product of step (c) to a temperature below −33° C.;

(e) heating the product of step (d) to a temperature above −13° C., under subatmospheric pressure for a time sufficient to remove water from the aqueous solvent and yield a solid lyophilized product.

Preferably, step (a) is conducted by dissolving in an aqueous solvent: a sPLA₂ inhibitor i.e., Sodium [[3-(2-amino-1,2-dioxoethyl)-2-ethyl-1-phenylmethyl)-1H-indol-4-yl]oxy]acetate; Solubilizer selected from citric acid, edetic acid, polyphosphoric acid and their salts, the amount of which is 1 to 100% (w/w) of the amount of the equivalent acid of Compound (Vb); and Stabilizer selected from mannitol, xylitol, sorbitol, glucose, fructose, lactose and maltose, the amount of which is 50 to 200% (w/w) of the equivalent acid of Compound (Vb). Moreover, each of steps (b), (c), (d) and (e) is preferably conducted for a period of at least one-half hour, and step (e) is performed at a subatmospheric pressure less than 133 Pa (1000 milliTorr).

Preferred parameters in the lyophilization process are those wherein Compound (Vb) for example, is frozen by cooling to −35° C. to −45° C. This cooling step is performed preferably over 2 to 4 hours. This process is herein after referred to as the “primary freezing process”. If desired, the frozen solution obtained in the primary freezing process is then warmed to −5° C. to −25° C. preferably from −10° C. to 20° C. This warming step is performed over 3 hours, preferably from 5 to 10 hours. This process is herein after referred to as the “heat treating process”.

The composition obtained in the heat treating process is re-frozen, preferably from −35° C. to −45° C. This cooling step is performed preferably over 2 to 4 hours. This process is herein after referred to as the “re-freezing process”.

The composition obtained through the primary freezing process, the heat treating process and the re-freezing process, is dried under a high vacuum by sublimating water according to methods known to those skilled in the art. Thus, a lyophilized preparation of the present invention is obtained. If desired, two step drying in which the temperature and the degree of vacuum are different may be performed for completely removing water. This process is herein after referred to as the “drying process”. If the two step drying is performed, these processes are referred to as the “primary drying” process and the “secondary drying” process.

The lyophilization process removes most of the water originally present, but the final product lyophilized composition may contain some free water. Typically, the water content can range from 0.5% to 5.0% weight percent. More typically, the water content ranges from 0.8% to 2.0%.

Inhalation therapy also may be useful either alone or as an adjunct to other routes of administration. With inhalation therapy, doses necessary to produce a decrease in the clinical symptoms of sepsis are readily determined and used.

Testing Methods for Sepsis

The diagnosis of sepsis is often inferential or suggestive. For example a patient exhibiting the symptoms of sepsis such as tachypenia, tachycardia, altered mental state, leukocytosis, or leukopenia and thrombocytopenia (Harrison's Principle of Internal Medicine supra). Because sepsis is believed to trigger the release of secretory phospholipase A₂, detection of increased or increasing levels of sPLA₂ in the plasma or systemic fluids is a useful indicia of the onset of sepsis or injurious conditions leading potentially to sepsis. Thus, another useful diagnostic tool may be the analysis of sPLA₂ levels over time to a patient determined to be susceptible to sepsis.

IL-6 levels are also somewhat useful separately or in combination with other methods as indicia for onset or susceptibility to sepsis.

Other diagnostic criteria for sepsis are those found in standard medical references (e.g., Harrison's Principles of Internal Medicine, thirteenth ed., 1994, pages 511-515, by McGraw-Hill, Inc., ISBN 0-07-032370-4), and in Sorensen, et al. Platelet Activating Factor and Phospholipase A₂ in Patients with Septic Shock and Trauma, Intensive Care Medicine (1994) 20, 555-561. These criteria, or criteria designated by competent medical opinion may be used to determine when to begin using the method of the invention, the frequency and degree of treatment, and the time for cessation of treatment.

| | |
|---|---|
| Investigators: | This multicenter study included 72 |
| | principal investigators. |
| Study Centers: | There were 72 study centers. |
| Dates of Study: | September 1998 through August 1999 |
| Clinical Phase: | Phase 2 |
| Objectives: | The primary objectives of this study |
| | were the following: to determine the |
| | optimal dose for Phase 3 testing based |
| | on a demonstrated reduction in 28-day |
| | all-cause mortality in patients with |
| | severe sepsis and to demonstrate that |
| | LY315920 has an acceptable safety |
| | profile. |
| Methodology: | Multicenter, double-blind, placebo- |
| | controlled trial of parallel design. |
| Number of | LY315920: Male 212, Female 178, Total |
| Subjects: | 390; |
| | Placebo: Male 116, Female 80, Total |
| | 196. |
| Diagnosis and | Males and females with severe sepsis, as |
| Inclusion | defined by meeting three or more of four |
| Criteria: | criteria for the systemic inflammatory |
| | response syndrome (SIRS) within the 36- |
| | hour time period immediately preceding |
| | entry into the study; suspected or |
| | proven presence of infection; at least |
| | one sepsis-induced organ failure within |
| | the 24-hour time period immediately |
| | preceding entry into the study. |
| Dosage and | Test Product-LY315920 sodium, supplied |
| Administration: | as a lyophilized powder. Each vial |
| | contained 100 mg LY315920. Following |
| | reconstitution, all doses of LY315920 |
| | were diluted in 5% dextrose for |
| | injection to a final concentration of |
| | 0.2 mg/mL or 0.8 mg/mL for intravenous |
| | infusions targeted to provide LY315920 |
| | plasma concentrations of approximately |
| | 200 and 800 ng/mL. The maximum volume |
| | of 5% dextrose to be administered within |
| | any 24-hour period could not exceed 600 mL. |
| | The study drug was administered |
| | intravenously via an infusion pump and |
| | was not to have been mixed with any |
| | other intravenous medication prior to |
| | administration. |
| | Placebo-A commercially available |
| | injectable multivitamin (Cernevit?, |
| | Baxter HealthCare Corporation). When |
| | mixed with 5% dextrose, the placebo |
| | solution appeared almost identical to a |
| | solution of LY315920. The amount of |
| | multivitamin administered was ≦ the |
| | daily recommended dose for the |
| | multivitamin preparation. |
| | CT11923: LY315920; CT14552: LY315920; |
| | CT14708: LY315920; CT9701149: placebo; |
| | CT9701150: placebo; |
| | CT9800291: placebo. |
| Duration of | LY315920 sodium: 7 days (168 hours) |
| Treatment: | |
| Criteria for: | Efficacy-The primary efficacy endpoint |
| Evaluation: | was the 28-day all-cause mortality. |
| | Patients were classified as either |
| | “alive at Day 28” or “dead at Day 28,” |
| | irrespective of the cause of death. The |
| | 28-day time point occurred at exactly |
| | 672 hours post start of study drug |
| | administration. |
| | Pharmacokinetics-The primary |
| | pharmacokinetic and pharmacodynamic |
| | measures were plasma LY315920 |
| | concentration, serum IL-6 concentration, |
| | serum sPLA₂ enzymatic activity, and |
| | serum sPLA₂ enzyme concentration. Each |
| | target plasma concentration was studied |
| | as a continuous 168 hour infusion. |
| | Safety-The following safety parameters |
| | were assessed in this study: adverse |
| | events (serious and treatment-emergent); |
| | vital signs; central laboratory tests; |
| | local laboratory tests; and incidence |
| | and severity of sepsis-associated organ |
| | dysfunction (SOFA scores). |
| Statistical | The data for qualitative variables were |
| Methods: | presented as incidence rates (number [N] |
| | and percent). For qualitative |
| | variables, treatment groups were |
| | compared using chi-square and Cochran- |
| | Mantel-Haenszel tests. Relative risk |
| | and confidence intervals were calculated |
| | using the logit adjusted relative risk |
| | method (SAS Institute Inc. 1989). All |
| | 28-day mortality analyses are based on |
| | chi-square and Cochran-Mantel-Haenszel |
| | tests. |
| | The data for continuous variables were |
| | summarized using measures of central |
| | tendency and dispersion. Statistical |
| | tests for continuous variables were |
| | performed using analysis of variance |
| | (ANOVA) based on ranked and unranked |
| | data (Snedecor and Cochran 1989). |
| | With the exception of the primary |
| | efficacy analysis, 2-sided 5% |
| | significance levels and 95% confidence |
| | intervals were used for all efficacy and |
| | safety analyses. The primary efficacy |
| | analysis 2-sided critical alpha level |
| | was 0.025, to account for two LY315920 |
| | dosing regimen comparisons to placebo. |

### Inclusion Criteria

Study participants were included in the study if they met all of the criteria listed below. It is anticipated that almost all patients meeting these criteria would have been in an acute care area of the hospital such as an intensive care unit, step-down unit, emergency ward, or recovery room. The events satisfying these inclusion criteria must have been attributable to the onset of sepsis and not attributable to an underlying disease process or to the effects of concomitant therapy.

[1] Meeting three or more of the following four criteria for SIRS within the 36-hour time period immediately preceding entry into the study. The criteria did not need to be present simultaneously.

A) Core temperature of >38° C. (100.4° F.) or <36° C. (96.8° F.); core temperature is defined as rectal, central catheter, or tympanic. If oral or axillary temperature is used, add 0.5° C. (1° F.) to the actual reading to determine qualification.

B) Heart rate≧90 beats per minute in the absence of known medical conditions or treatments that would prevent tachycardia (for example, use of beta adrenergic blockers or heart block). If patients had a known medical condition or were receiving treatment that would prevent tachycardia, they must have met two of the remaining three criteria for systemic inflammation.

C) Respiratory rate≧20 breaths per minute or PaCO₂≦32 mm Hg or mechanical ventilation for an acute process (that was not related to a neuromuscular disease or the need for general anesthesia).

D) White blood cell count≧12,000 or ≦4000 cells/mm³ (not secondary to oncolytic agents) or >10% immature neutrophils (bands).

[2] Suspected or proven presence of infection. Patients with suspected infection must have had evidence of an acute infection such as white blood cells in a normally sterile body fluid, perforated viscus, chest X-ray consistent with pneumonia and associated with purulent sputum production, or a clinical syndrome associated with a high probability of infection (for example, ascending cholangitis).

[3] Have had at least one of the following sepsis-induced organ failures within the 24-hour time period immediately preceding entry into the study:

A) Cardiovascular: a) An arterial systolic blood pressure of <90 mm Hg for at least one hour despite adequate fluid resuscitation or the administration of an intravenous fluid bolus (>500 mL of normal saline or equivalent over one hour); or b) A requirement for vasopressor administration for at least one hour to maintain a systolic blood pressure≧90 mm Hg. Vasopressors include dopamine (≧5 μg/kg/min) and phenylephrine, epinephrine, or norepinephrine at any dose. Dobutamine is not considered a vasopressor. and/or

B) Respiratory: a) Evidence of acute pulmonary dysfunction defined as a PaO₂/FiO₂ ratio of ≦300 and (if measured) a pulmonary capillary wedge pressure not suggestive of central volume overload; or b) In the setting of pneumonia, the patient must have had a PaO₂/FiO₂ ratio≦200 and (if measured) a pulmonary capillary wedge pressure not suggestive of central volume overload. and/or

C) Hematology: a) Platelet count<100,000/mm³; or b) 50% decrease in the platelet count from the highest value recorded over the three days immediately preceding screening. and/or

D) Renal: a) Urine output<0.5 mL/kg/hr for two consecutive hours despite adequate fluid resuscitation; or b) A serum creatinine concentration>3.0 mg/dL. (In the presence of preexisting renal impairment, defined as a serum creatinine concentration>3.0 mg/dL prior to the onset of the sepsis episode, the patient must have met one of the other four organ failures.) and/or

E) Lactic acidosis characterized by an elevation in arterial plasma lactic acid concentration>1.5 times the upper limit of normal and associated with an arterial pH<7.3 or a base deficit>5.0 mEq/L.

### Drug Concentration Measurements

The primary pharmacokinetic and pharmacodynamic measures were: 1) plasma LY315920 concentration; 2) serum IL-6 concentration; 3) serum sPLA₂ enzymatic activity; and 4) serum sPLA₂ enzyme concentration. One plasma sample (LY315920) and one serum sample (sPLA₂ activity, sPLA₂ concentration, IL-6 concentration) was collected from each patient during at specified time intervals. An additional plasma specimen (citrate anticoagulant) was obtained with the pharmacokinetic and pharmacodynamic samples. The latter specimen was stored frozen for future evaluation. All specimens were to have been collected at the same time. The exact clock time of sample collection was recorded. Care was taken that the plasma sample for determination of LY315920 concentration was obtained free of possible contamination from infusion solutions.

Results

The results showed a statistically significant reduction in mortality when patients with sepsis were treated with a sPLA₂ compound of formula I, i.e., compound of formula (Vb) (LY315920) within 24 hours after first organ failure but preferably within 18 hours after first organ failure and by implication most preferably within 12 hours after first organ failure. By extrapolation the results indicate that initiation of administration of a sPLA₂ inhibitor compound to a patient susceptible to sepsis is most preferred. The results of baseline analyses of IL-6 levels and sPLA₂ activity levels are shown in tables 1 through 3. The mortality data from the study are shown in diagrams 1 through 3.

TABLE 1
Baseline IL-6 concentration
by time from first organ failure to first dose of study drug
and gender

| | N of | | | | |
|---|---|---|---|---|---|
| | patients | Mean | Maximum | Median | Minimum |
| All patients | | | | | |
| <12 hours | 69 | 8125 | 101150 | 697 | 14.35 |
| 12-24 hours | 159 | 6175 | 296550 | 458 | 6.30 |
| 24-48 hours | 217 | 6264 | 479000 | 358 | 5.14 |
| >48 hours | 119 | 1051 | 29074 | 190 | 12.40 |
| Females | | | | | |
| <12 hours | 33 | 9693 | 64730 | 1056 | 14.35 |
| 12-24 hours | 81 | 5216 | 170250 | 482 | 11.26 |
| 24-48 hours | 89 | 5673 | 223800 | 400 | 5.14 |
| >48 hours | 45 | 650 | 11576 | 169 | 12.40 |
| Males | | | | | |
| <12 hours | 36 | 6687 | 101150 | 630 | 27.50 |
| 12-24 hours | 78 | 7170 | 296550 | 327 | 6.30 |
| 24-48 hours | 128 | 6675 | 479000 | 333 | 5.98 |
| >48 hours | 74 | 1295 | 29074 | 195 | 13.92 |

TABLE 2
Baseline IL-6 concentration
by number of organ failures at baseline and gender

| | N of | | | | |
|---|---|---|---|---|---|
| | patients | Mean | Maximum | Median | Minimum |
| All patients | | | | | |
| 0-1 failures | 126 | 1644 | 50350 | 191 | 5.14 |
| 2 failures | 196 | 2144 | 170250 | 233 | 11.26 |
| 3 failures | 151 | 6894 | 296550 | 606 | 16.50 |
| 4-5 failures | 91 | 14929 | 479000 | 767 | 18.65 |
| Females | | | | | |
| 0-1 failures | 47 | 2161 | 50350 | 245 | 5.14 |
| 2 failures | 83 | 3180 | 170250 | 304 | 11.26 |
| 3 failures | 68 | 5287 | 51700 | 729 | 26.65 |
| 4-5 failures | 50 | 11031 | 223800 | 636 | 18.65 |
| Males | | | | | |
| 0-1 failures | 79 | 1336 | 48965 | 183 | 5.98 |
| 2 failures | 113 | 1383 | 46300 | 203 | 17.70 |
| 3 failures | 83 | 8211 | 296550 | 568 | 16.50 |
| 4-5 failures | 41 | 19682 | 479000 | 1179 | 41.70 |

TABLE 3
sPLA₂ activity and duration of first sepsis induced organ
failure

| | N of | | | | |
|---|---|---|---|---|---|
| | Patients | Mean | Maximum | Median | Minimum |
| All patients | | | | | |
| <12 hours | 62 | 289 | 2400 | 145 | 0 |
| 12-24 hours | 124 | 471 | 2400 | 305 | 0 |
| 24-48 hours | 176 | 418 | 2400 | 246 | 0 |
| >48 hours | 90 | 314 | 143 | 162 | 0 |
| Females | | | | | |
| <12 hours | 30 | 360 | 2400 | 153 | 0 |
| 12-24 hours | 61 | 473 | 2400 | 210 | 0 |
| 24-48 hours | 71 | 428 | 2400 | 260 | 0 |
| >48 hours | 36 | 251 | 1142 | 145 | 0 |
| Males | | | | | |
| <12 hours | 32 | 223 | 1216 | 125 | 0 |
| 12-24 hours | 63 | 469 | 1949 | 358 | 0 |
| 24-48 hours | 105 | 412 | 2400 | 237 | 0 |
| >48 hours | 54 | 356 | 1434 | 219 | 0 |

While the present invention has been illustrated above by certain specific embodiments, it is not intended that these specific examples should limit the scope of the invention as described in the appended claims.

We claim:

1. A method of preventing sepsis in a mammal including a human, said method comprising initiating administration to a patient susceptible to sepsis a pharmaceutically effective amount of a sPLA₂ inhibitor compound prior to occurrence of injury using conditions.

2. A method of treating sepsis wherein treatment of a patient with a pharmaceutically effective amount of a sPLA₂ inhibitor compound of formula I or II is initiated within a time interval from first organ failure or onset of rise in sPLA₂ activity levels.

3. A method of treating sepsis wherein treatment of a patient with a pharmaceutically effective amount of a sPLA₂ inhibitor compound of formula I or II or a pharmaceutically acceptable salt, solvate or prodrug derivative thereof, is initiated within a time interval from first organ failure or onset of elevated sPLA₂ levels.

4. A method according to claim 2 wherein the time interval is from 0 to 24 hours after first organ failure.

5. A method according to claim 2 wherein the time interval is from 0 to 24 hours after first organ failure or the onset of elevated sPLA₂ levels.

6. A method according to claim 2 wherein the time interval is from 0 to 18 hours after first organ failure or the onset of elevated sPLA₂ levels.

7. A method according to claim 2 wherein the time interval is from 0 to 12 hours after first organ failure or the onset of elevated sPLA₂ levels.

8. A method according to claim 2 wherein the time interval is from 0 to 8 hours after first organ failure or the onset of elevated sPLA₂ levels.

9. A method according to claim 2 wherein the time interval is from 0 to 6 hours after first organ failure or the onset of elevated sPLA₂ levels.

10. A method according to claim 1 or 2 or 3 or 4 or 5 or 6 or 7 wherein the sPLA₂ inhibitor compound of formula I is; 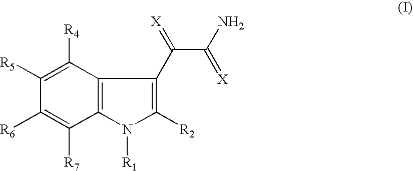
where;
X is oxygen,
R¹ is selected from the group consisting of —C₇-C₂₀ alkyl, 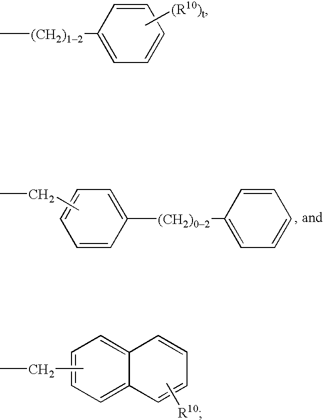
where
R¹⁰ is selected from the group consisting of halo, C₁-C₁₀ alkyl, C₁-C₁₀ alkoxy, —S—(C₁-C₁₀ alkyl) and halo(ClC₁₀)alkyl, and t is an integer from 0 to 5 both inclusive;
R₂ is selected from the group consisting of hydrogen, halo, cyclopropyl, methyl, ethyl, and propyl;
R₄ and R₅ are independently selected from the group consisting of hydrogen, a non-interfering substituent and the group, -(Lₐ)-(acidic group); where,
at least one of R₄ and R₅ is the group, -(Lₐ)-(acidic group) and wherein the (acidic group) is selected from the group consisting of —CO₂H, —SO₃H, or —P(O)(OH)₂; where,
-(Lₐ)- is an acid linker with the proviso that;
the acid linker group, -(Lₐ)-, for R₄ is selected from the group consisting of 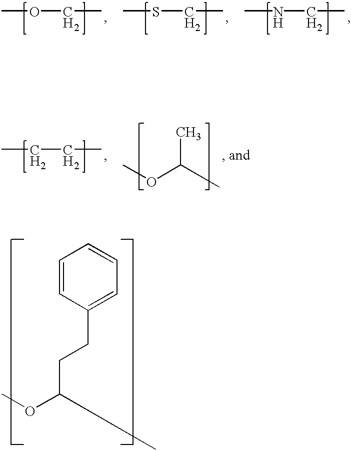
where R¹⁰³ is a non-interfering substituent, and where,
the acid linker, -(Lₐ)-, for R⁵ is selected from the group consisting of 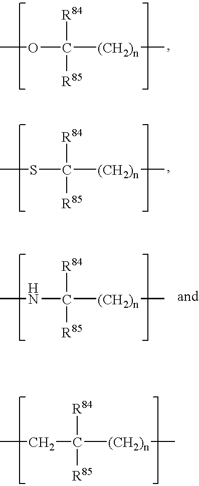
where R⁸⁴ and R⁸⁵ are each independently selected from hydrogen, C₁-C₁₀ alkyl, aryl, C₁-C₁₀ alkaryl, C₁-C₁₀ arylkyl, carboxy, carbalkoxy, and halo and,
R₆ and R₇ are each independently selected from hydrogen and non-interfering substituents, where non-interfering substituents are selected from the group consisting of C₁-C₆ alkyl, C₂-C₆ alkenyl, C₂-C₆ alkynyl, C₇-C₁₂ arylenalkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between 1 and 8.

11. A method according to claim 1 or 2 or 3 or 4 or 5 or 6 or 7 wherein the sPLA₂ inhibitor compound is a compound of formula I or a pharmaceutically acceptable salt, solvate or prodrug derivative thereof: 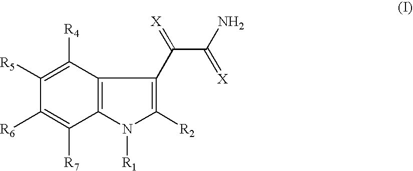
where;
X is oxygen,
R¹ is selected from the group consisting of —C₇-C₂₀ alkyl, 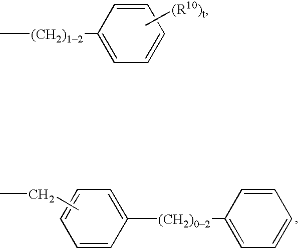
where
R¹⁰ is selected from the group consisting of halo, C₁-C₁₀alkyl, C₁-C₁₀ alkoxy, —S—(C₁-C₁₀ alkyl) and halo(C₁-C₁₀)alkyl, and t is an integer from 0 to 5 both inclusive;
R₂ is selected from the group consisting of hydrogen, halo, cyclopropyl, methyl, ethyl, and propyl;
R₄ and R₅ are independently selected from the group consisting of hydrogen, a non-interfering substituent and the group, -(Lₐ)-(acidic group); where,
at least one of R₄ and R₅ is the group, -(Lₐ)-(acidic group) and wherein the (acidic group) is selected from the group consisting of —CO₂H, —SO₃H, or —P(O)(OH)₂; where,
-(Lₐ)- is an acid linker with the proviso that;
the acid linker group, -(Lₐ)-, for R₄ is selected from the group consisting of 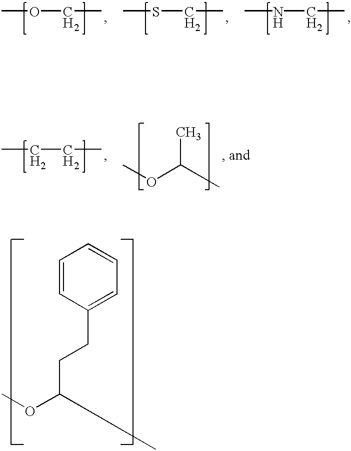
where R¹⁰³ is a non-interfering substituent, and where,
the acid linker, -(Lₐ)-, for R⁵ is selected from the group consisting of 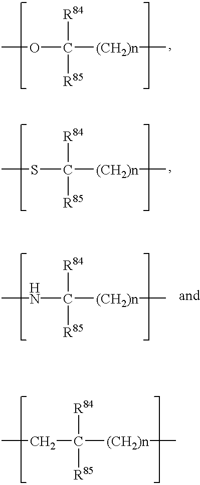
where R⁸⁴ and R⁸⁵ are each independently selected from hydrogen, C₁-C₁₀ alkyl, aryl, C₁-C₁₀ alkaryl, C₁-C₁₀ arylkyl, carboxy, carbalkoxy, and halo and,
R₆ and R₇ are each independently selected from hydrogen and non-interfering substituents, where non-interfering substituents are selected from the group consisting of C₁-C₆ alkyl, C₂-C₆ alkenyl, C₂-C₆ alkynyl, C₇-C₁₂ arylenalkyl, C₇-C₁₂ alkaryl, C₃-C₈ cycloalkyl, C₃-C₉ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between 1 and 8; and R is hydrogen, C₁-C₆ alkyl.

12. A method according to claim 1 or 2 or 3 or 4 or 5 or 6 or 7 wherein the sPLA₂ inhibitor compound of formula II is: 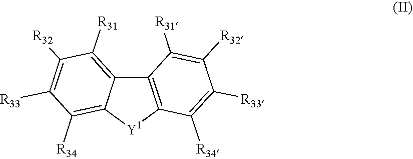
where Y¹ is selected from the group consisting of O, NH, NR¹ and S;
R¹ is selected from the group consisting of —C₇-C₂₀ alkyl, 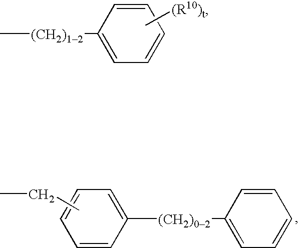
where
R¹⁰ is selected from the group consisting of halo, C₁Clo alkyl, C₁-C₁₀ alkoxy, —S—(C₁-C₁₀ alkyl) and halo(C₁-C₁₀)alkyl, and t is an integer from 0 to 5 both inclusive;
where R₃₁, R₃₂, R₃₃, R₃₁1, R₃₂1, R₃₃1, R₃₄ and R₃₄1 are independently selected from the group consisting of hydrogen, CONR¹⁰¹R¹⁰², alkyl, alkylaryl, aryl, alkylheteroaryl, haloalkyl, alkylCONR¹⁰¹R¹⁰², a non-interfering substituent and the group, -(Lₐ)-(acidic group);
where -(Lₐ)- is an acid linker selected from the group consisting of 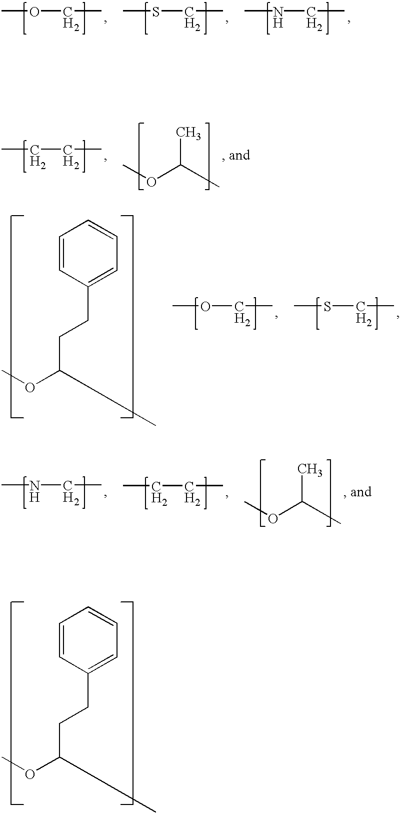
where R⁸⁴ and R⁸⁵ are each independently selected from the group consisting of hydrogen, C₁-C₁₀ alkyl, aryl, C₁-C₁₀ alkaryl, C₁-C₁₀ aralkyl, carboxy, carbalkoxy, and halo; and n is 1 or 2 and,
where the (acidic group) is selected from the group consisting of —CO₂H, —SO₃H, and —P(O)(OH)₂ and,
where R¹⁰¹ and R¹⁰² are independently selected from the group consisting of hydrogen, alkyl, aryl, heteroaryl and haloalkyl and,
where non-interfering substituents are selected from the group consisting of C₁-C₆ alkyl, C₂-C₆ alkenyl, C₂-C₆ alkynyl, C₇-C₁₂ arylalkyl, C₇-C₁₂ alkylaryl, C₃-C₈ cycloalkyl, C₃-C₈ cycloalkyl, phenyl, tolulyl, xylyl, biphenyl, C₁-C₆ alkoxy, C₂-C₆ alkyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, C(O)O(C₁-C₆ alkyl), —(CH₂)ₙ—O—(C₁-C₆ alkyl), benzyloxy, phenoxy, phenylthio, —(CONHSO₂(R)), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between about 1 and 8 and,
R is selected from the group consisting of hydrogen and alkyl and,
where at least one of R₃₁, R₃₂, R₃₃ or R₃₄ is the group -(Lₐ)-(acidic group).

13. The method of claim 1 or 2 or 3 or 4 or 5 or 6 or 7 wherein the compound of formula I or II is in a pharmaceutical formulation comprising the compound of formula I or II in combination with a carrier or diluent.

14. The method of claim 1 or 2 or 3 or 4 or 5 or 6 or 7 or 8 or 9 wherein the compound of formula I or II or a pharmaceutically acceptable salt, solvate or prodrug thereof is in a pharmaceutical formulation comprising the compound of formula I or II or a pharmaceutically acceptable salt, solvate or prodrug thereof in combination with a carrier or diluent.

15. The method of claim 11 wherein the pharmaceutical formulation comprises the freeze dried lyophilized formulation of a compound of formula I or II or a pharmaceutically acceptable salt, solvate or prodrug derivative thereof.

16. The method of claim 11 wherein the pharmaceutical formulation comprises the freeze dried lyophilized formulation of a compound of formula (Vb) shown below:

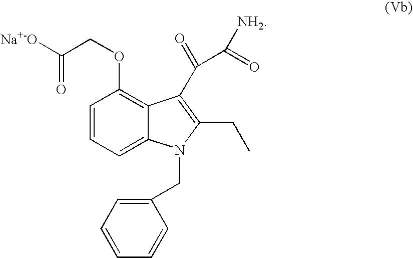

17. The method of claim 1 or 2 or 3 or 4 or 5 or 6 or 7 wherein the compound of formula I or II is in a pharmaceutical formulation comprising the compound of formula I or II in combination with other effective drug for the treatment of sepsis, a carrier and/or diluent.

18. The method of claim 1 or 2 or 3 or 4 or 5 or 6 or 7 wherein the compound of formula I or II or a pharmaceutically acceptable salt, solvate or prodrug thereof is in a pharmaceutical formulation comprising the compound of formula I or II or a pharmaceutically acceptable salt, solvate or prodrug thereof in combination with other effective drug for the treatment of sepsis, a carrier and/or diluent.

19. A method according to claim 1 or 2 or 3 or 4 or 5 or 6 or 7 wherein the compound of formula I is selected from the group consisting of:
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-(phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([11′-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid
(G) [[3-(2-Amino-1,2-dioxoethyl)-1-[4(fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-[(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl]oxy]butanoic acid,
mixtures of (A) through (P) in any combination or a pharmaceutically acceptable racemate, solvate, tautomer, optical isomer, prodrug derivative or salt, thereof.

20. A method according to claim 1 or 2 or 3 or 4 or 5 or 6 or 7 wherein the compound of formula II is selected from the group consisting of:
9-benzyl-5,7-dimethoxy-1,2,3,4-tetrahydrocarbazole-4-carboxylic acid hydrazide;
9-benzyl-5,7-dimethoxy-1,2,3,4-tetrahydrocarbazole-4-carboxamide;
[9-benzyl-4-carbamoyl-7-methoxy-1,2,3,4-tetrahydrocarbazol-5-yl]oxyacetic acid sodium salt;
[9-benzyl-4-carbamoyl-7-methoxycarbazol-5-yl]oxyacetic acid;
Methyl [9-benzyl-4-carbamoyl-7-methoxycarbazol-5-yl]oxyacetic acid;
9-benzyl-7-methoxy-5-cyanomethyloxy-1,2,3,4-tetrahydrocarbazole-4-carboxamide;
9-benzyl-7-methoxy-5-(1H-tetrazol-5-yl-methyl)oxy)-1,2,3,4-tetrahydrocarbazole-4-carboxamide;
{9-[(phenyl)methyl]-5-carbamoyl-2-methyl-carbazol-4-yl}oxyacetic acid;
{9-[(3-fluorophenyl)methyl]-5-carbamoyl-2-methyl-carbazol-4-yl}oxyacetic acid;
{9-[(3-methylphenyl)methyl]-5-carbamoyl-2-methyl-carbazol-4-yl}oxyacetic acid;
{9-[(phenyl)methyl]-5-carbamoyl-2-(4-trifluoromethylphenyl)carbazol-4-yl}oxyacetic acid;
9-benzyl-5-(2-methanesulfonamido)ethyloxy-7-methoxy-1,2,3,4-tetrahydrocarbazole-4-carboxamide;
9-benzyl-4-(2-methanesulfonamido)ethyloxy-2-methoxycarbazole-5-carboxamide;
9-benzyl-4-(2-trifluoromethanesulfonamido)ethyloxy-2-methoxycarbazole-5-carboxamide;
9-benzyl-5-methanesulfonamidoylmethyloxy-7-methoxy-1,2,3,4-tetrahydrocarbazole-4-carboxamide;
9-benzyl-4-methanesulfonamidoylmethyloxy-carbazole-5-carboxamide;
[5-carbamoyl-2-pentyl-9-(phenylmethyl)carbazol-4-yl]oxyacetic acid;
[5-carbamoyl-2-(1-methylethyl)-9-(phenylmethyl)carbazol-4-yl]oxyacetic acid;
[5-carbamoyl-9-(phenylmethyl)-2-[(tri(−1-methylethyl)silyl)oxymethyl]carbazol-4-yl]oxyacetic acid;
[5-carbamoyl-2-phenyl-9-(phenylmethyl)carbazol-4-yl]oxyacetic acid;
[5-carbamoyl-2-(4-chlorophenyl)-9-(phenylmethyl)carbazol-4-yl]oxyacetic acid;
[5-carbamoyl-2-(2-furyl)-9-(phenylmethyl)carbazol-4-yl]oxyacetic acid;
[5-carbamoyl-9-(phenylmethyl)-2-[(tri(−1-methylethyl)silyl)oxymethyl]carbazol-4-yl]oxyacetic acid, lithium salt;
{9-[(phenyl)methyl]-5-carbamoylcarbazol-4-yl}oxyacetic acid;
{9-[(3-fluorophenyl)methyl]-5-carbamoylcarbazol-4-yl}oxyacetic acid;
{9-[(3-phenoxyphenyl)methyl]-5-carbamoylcarbazol-4-yl}oxyacetic acid;
{9-[(2-Fluorophenyl)methyl]-5-carbamoylcarbazol-4-yl}oxyacetic acid;
{9-[(2-trifluoromethylphenyl)methyl]-5-carbamoylcarbazol-4-yl}oxyacetic acid;
{9-[(2-benzylphenyl)methyl]-5-carbamoylcarbazol-4-yl}oxyacetic acid;
{9-[(3-trifluoromethylphenyl)methyl]-5-carbamoylcarbazol-4-yl}oxyacetic acid;
{9-[(1-naphthyl)methyl]-5-carbamoylcarbazol-4-yl}oxyacetic acid;
{9-[(2-cyanophenyl)methyl]-5-carbamoylcarbazol-4-yl}oxyacetic acid;
{9-[(3-cyanophenyl)methyl]-5-carbamoylcarbazol-4-yl}oxyacetic acid;
{9-[(2-methylphenyl)methyl]-5-carbamoylcarbazol-4-yl}oxyacetic acid;
{9-[(3-methylphenyl)methyl]-5-carbamoylcarbazol-4-yl}oxyacetic acid;
{9-[(3,5-dimethylphenyl)methyl]-5-carbamoylcarbazol-4-yl}oxyacetic acid;
{9-[(3-iodophenyl)methyl]-5-carbamoylcarbazol-4-yl}oxyacetic acid;
{9-[(2-Chlorophenyl)methyl]-5-carbamoylcarbazol-4-yl}oxyacetic acid;
{9-[(2,3-difluorophenyl)methyl]-5-carbamoylcarbazol-4-yl}oxyacetic acid;
{9-[(2,6-difluorophenyl)methyl]-5-carbamoylcarbazol-4-yl}oxyacetic acid;
{9-[(2,6-dichlorophenyl)methyl]-5-carbamoylcarbazol-4-yl}oxyacetic acid;
{9-[(3-trifluoromethoxyphenyl)methyl]-5-carbamoylcarbazol-4-yl}oxyacetic acid;
{9-[(2-biphenyl)methyl]-5-carbamoylcarbazol-4-yl}oxyacetic acid;
{9-[(2-Biphenyl)methyl]-5-carbamoylcarbazol-4-yl}oxyacetic acid;
the {9-[(2-Biphenyl)methyl]-5-carbamoylcarbazol-4-yl}oxyacetic acid;
[9-Benzyl-4-carbamoyl-1,2,3,4-tetrahydrocarbaole-5-yl]oxyacetic acid;
{9-[(2-Pyridyl)methyl]-5-carbamoylcarbazol-4-yl}oxyacetic acid;
{9-[(3-Pyridyl)methyl]-5-carbamoylcarbazol-4-yl}oxyacetic acid;
[9-benzyl-4-carbamoyl-8-methyl-1,2,3,4-tetrahydrocarbazol-5-yl]oxyacetic acid;
[9-benzyl-5-carbamoyl-1-methylcarbazol-4-yl]oxyacetic acid;
[9-benzyl-4-carbamoyl-8-fluoro-1,2,3,4-tetrahydrocarbazol-5-yl]oxyacetic acid;
[9-benzyl-5-carbamoyl-1-fluorocarbazol-4-yl]oxyacetic acid;
[9-benzyl-4-carbamoyl-8-chloro-1,2,3,4-tetrahydrocarbazol-5-yl]-oxyacetic acid;
[9-benzyl-5-carbamoyl-1-chlorocarbazol-4-yl]oxyacetic acid;
[9-[(Cyclohexyl)methyl]-5-carbamoylcarbazol-4-yl]oxyacetic acid;
[9-[(Cyclopentyl)methyl]-5-carbamoylcarbazol-4-yl]oxyacetic acid;
5-carbamoyl-9-(phenylmethyl)-2-[[(propen-3-yl)oxy]methyl]carbazol-4-yl]oxyacetic acid;
[5-carbamoyl-9-(phenylmethyl)-2-[(propyloxy)methyl]carbazol-4-yl]oxyacetic acid;
9-benzyl-7-methoxy-5-((carboxamidomethyl)oxy)-1,2,3,4-tetrahydrocarbazole-4-carboxamide;
9-benzyl-7-methoxy-5-cyanomethyloxy-carbazole-4-carboxamide;
9-benzyl-7-methoxy-5-((1H-tetrazol-5-yl-methyl)oxy)carbazole-4-carboxamide;
9-benzyl-7-methoxy-5-((carboxamidomethyl)oxy)-carbazole-4-carboxamide; and
[9-Benzyl-4-carbamoyl-1,2,3,4-tetrahydrocarbaole-5-yl]oxyacetic-acid
or a pharmaceutically acceptable racemate, solvate, tautomer, optical isomer-, prodrug derivative, or salt thereof.

21. A method of claim 1 or 2 or 3 or 4 or 5 or 6 or 7 wherein the compound of formula I or II is selected from the group consisting of: 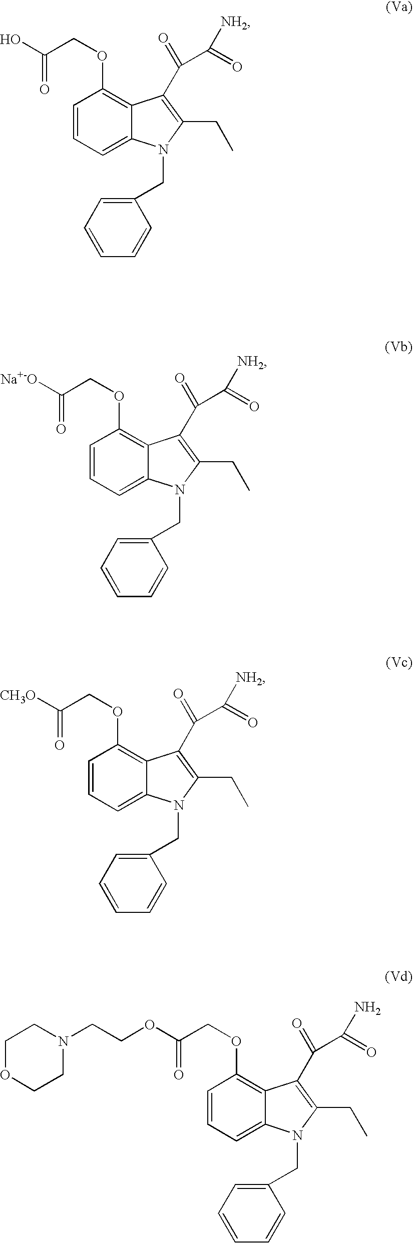 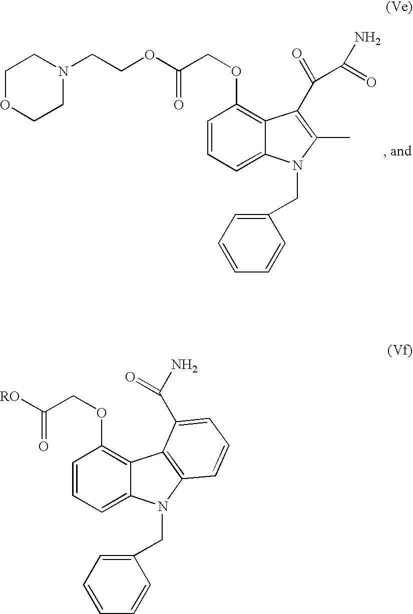
wherein R is methyl, ethyl, sodium ion, or N-morpholinoethyl group.

22. A method according to claim 1, 2, 3, 4, 5, 6, or 7, wherein the compound is

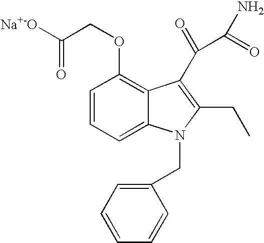

23. A method according to claim 1 comprising administration of a combination of sPLA₂ inhibitor compound and other effective therapy for sepsis.

24. A method according to claim 12 wherein the other effective therapy for sepsis is Activated Protein C or N-[o-(p-pivaloyloxybenzene)sulfonylaminobenzoyl]glycine.

25. A method preventing or treating sepsis comprising the steps of:
c. selecting patient susceptible to sepsis;
d. monitoring sPLA₂ activity levels in patient;
e. administering effective amount of a compound of formula I or II if sPLA₂ activity levels are high or on the rise.

26. A method treating sepsis comprising the steps of:
a. selecting a patient, afflicted with sepsis within 18 hours after first organ failure;
b. initiating administration of effective amount of a compound of formula I or II;
c. continuing administration of effective amount of a compound of formula I or II for about 1 to 7 days thereafter or until a medically determined stopping point or sPLA₂ activity levels normalize.

27. A method treating sepsis comprising the steps of:
a. selecting a patient afflicted with sepsis within 12 hours after first organ failure;
b. initiating administration of effective amount of a compound of formula I or II or a pharmaceutically acceptable salt, solvate or prodrug thereof.

28. A method treating sepsis comprising the steps of:
a. selecting a patient afflicted with sepsis within 6 hours after first organ failure;
b. initiating administration of effective amount of a compound of formula I or II.

29. Use of a sPLA₂ inhibitor compound of formula I or II or a pharmaceutically acceptable salt, solvate or prodrug derivative thereof, for the manufacture of a medicament for the treatment of sepsis wherein administration of the pharmaceutically effective amount of a sPLA₂ inhibitor compound of formula I or II or a pharmaceutically acceptable salt, solvate or prodrug derivative thereof, is initiated within a time interval from first organ failure or onset of elevated sPLA₂ levels.

30. Use of a compound of formula (I) or (II) in the manufacture of a medicament for the treatment or prevention of sepsis in a patient afflicted with sepsis or susceptible to sepsis comprising initiating administration of a pharmaceutical formulation comprising a compound of formula I or II within 24 hours after first organ failure or prior to a rise in sPLA₂ levels.

31. Use of a compound of formula (I) or (II) in the manufacture of a medicament for the treatment or prevention of sepsis in a patient afflicted with sepsis or susceptible to sepsis comprising initiating administration of a pharmaceutical formulation comprising a compound of formula I or II in combination with other effective therapy or co-agent for sepsis within 24 hours after first organ failure or prior to a rise in sPLA₂ levels.

32. Use of a compound of formula (I) or (II) in the manufacture of a medicament for the treatment or prevention of sepsis according to claim 30 wherein the time interval is from 0 to 18 hours after first organ failure or the onset of elevated sPLA₂ levels.

33. Use of a compound of formula (I) or (II) in the manufacture of a medicament for the treatment or prevention of sepsis according to claim 30 wherein the time interval is from 0 to 12 hours after first organ failure or the onset of elevated sPLA₂ levels.

34. Use of a compound of formula (I) or (II) in the manufacture of a medicament for the treatment or prevention of sepsis according to claim 30 wherein the time interval is from 0 to 8 hours after first organ failure or the onset of elevated sPLA₂ levels.

35. Use of a compound of formula (I) or (II) in the manufacture of a medicament for the treatment or prevention of sepsis according to claim 30 wherein the time interval is from 0 to 6 hours after first organ failure or the onset of elevated sPLA₂ levels.

36. Use of a compond of formula I or II according to the method of claim 1, 2, 3, 4, 5, 6, or 7, for the manufacture of a medicament for the treatment of sepsis wherein the compound of formula I is selected from the group consisting of:
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([111-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid
(G) [13-(2-Amino-1,2-dioxoethyl)-1-[4-(fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl)-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-1-(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl]oxy]butanoic acid,
mixtures of (A) through (P) in any combination or a pharmaceutically acceptable racemate, solvate, tautomer, optical isomer, prodrug derivative or salt, thereof.

37. Use of a compound of formula I or II according to the method of claim 1, 2, 3, 4, 5, 6, or 7, for the manufacture of a medicament for the treatment of sepsis wherein the compound of formula II is selected from the group consisting of:
9-benzyl-5,7-dimethoxy-1,2,3,4-tetrahydrocarbazole-4-carboxylic acid hydrazide;
9-benzyl-5,7-dimethoxy-1,2,3,4-tetrahydrocarbazole-4-carboxamide;
[9-benzyl-4-carbamoyl-7-methoxy-1,2,3,4-tetrahydrocarbazol-5-yl]oxyacetic acid sodium salt;
[9-benzyl-4-carbamoyl-7-methoxycarbazol-5-yl]oxyacetic acid;
Methyl 19-benzyl-4-carbamoyl-7-methoxycarbazol-5-yl]oxyacetic acid;
9-benzyl-7-methoxy-5-cyanomethyloxy-1,2,3,4-tetrahydrocarbazole-4-carboxamide;
9-benzyl-7-methoxy-5-(1H-tetrazol-5-yl-methyl)oxy)-1,2,3,4-tetrahydrocarbazole-4-carboxamide;
{9-[(phenyl)methyl]-5-carbamoyl-2-methyl-carbazol-4-yl}oxyacetic acid;
{9-[(3-fluorophenyl)methyl]-5-carbamoyl-2-methyl-carbazol-4-yl}oxyacetic acid;
{9-[(3-methylphenyl)methyl]-5-carbamoyl-2-methyl-carbazol-4-yl}oxyacetic acid;
{9-[(phenyl)methyl]-5-carbamoyl-2-(4-trifluoromethylphenyl)-carbazol-4-yl}oxyacetic acid;
9-benzyl-5-(2-methanesulfonamido)ethyloxy-7-methoxy-1,2,3,4-tetrahydrocarbazole-4-carboxamide;
9-benzyl-4-(2-methanesulfonamido)ethyloxy-2-methoxycarbazole-5-carboxamide;
9-benzyl-4-(2-trifluoromethanesulfonamido)ethyloxy-2-methoxycarbazole-5-carboxamide;
9-benzyl-5-methanesulfonamidoylmethyloxy-7-methoxy-1,2,3,4-tetrahydrocarbazole-4-carboxamide;
9-benzyl-4-methanesulfonamidoylmethyloxy-carbazole-5-carboxamide;
[5-carbamoyl-2-pentyl-9-(phenylmethyl)carbazol-4-yl]oxyacetic acid;
[5-carbamoyl-2-(1-methylethyl)-9-(phenylmethyl)carbazol-4-yl]oxyacetic acid;
[5-carbamoyl-9-(phenylmethyl)-2-[(tri(−1-methylethyl)silyl)oxymethyl]carbazol-4-yl]oxyacetic acid;
[5-carbamoyl-2-phenyl-9-(phenylmethyl)carbazol-4-yl]oxyacetic acid[5-carbamoyl-2-(4-chlorophenyl)-9-(phenylmethyl)carbazol-4-yl]oxyacetic acid;
[5-carbamoyl-2-(2-furyl)-9-(phenylmethyl)carbazol-4-yl]oxyacetic acid;
[5-carbamoyl-9-(phenylmethyl)-2-[(tri(-1-methylethyl)silyl)oxymethyl]carbazol-4-yl]oxyacetic acid, lithium salt;
{9-[(phenyl)methyl]-5-carbamoylcarbazol-4-yl}oxyacetic acid;
{9-[(3-fluorophenyl)methyl]-5-carbamoylcarbazol-4-yl}oxyacetic acid;
{9-[(3-phenoxyphenyl)methyl]-5-carbamoylcarbazol-4-yl}oxyacetic acid;
{9-[(2-Fluorophenyl)methyl]-5-carbamoylcarbazol-4-yl}oxyacetic acid;
{9-[(2-trifluoromethylphenyl)methyl]-5-carbamoylcarbazol-4-yl}oxyacetic acid;
{9-[(2-benzylphenyl)methyl]-5-carbamoylcarbazol-4-yl}oxyacetic acid;
{9-[(3-trifluoromethylphenyl)methyl]-5-carbamoylcarbazol-4-yl}oxyacetic acid;
{9-[(1-naphthyl)methyl]-5-carbamoylcarbazol-4-yl}oxyacetic acid;
{9-[(2-cyanophenyl)methyl]-5-carbamoylcarbazol-4-yl}oxyacetic acid;
{9-[(3-cyanophenyl)methyl]-5-carbamoylcarbazol-4-yl}oxyacetic acid;
{9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid;
{9-[(3,5-dimethylphenyl)methyl]-5-carbamoylcarbazol-4-yl}oxyacetic acid;
{9-[(3-iodophenyl)methyl]-5-carbamoylcarbazol-4-yl}oxyacetic acid;
{9-[(2-Chlorophenyl)methyl]-5-carbamoylcarbazol-4-yl}oxyacetic acid;
{9-[(2,3-difluorophenyl)methyl]-5-carbamoylcarbazol-4-yl}oxyacetic acid;
{9-[(2,6-difluorophenyl)methyl]-5-carbamoylcarbazol-4-yl}oxyacetic acid;
{9-[(2,6-dichlorophenyl)methyl]-5-carbamoylcarbazol-4-yl}oxyacetic acid;
{9-[(3-trifluoromethoxyphenyl)methyl]-5-carbamoylcarbazol-4-yl}oxyacetic acid;
{9-[(2-biphenyl)methyl]-5-carbamoylcarbazol-4-yl}oxyacetic acid;
{9-[(2-Biphenyl)methyl]-5-carbamoylcarbazol-4-yl}oxyacetic acid;
the {9-[(2-Biphenyl)methyl]-5-carbamoylcarbazol-4-yl}oxyacetic acid;
[9-Benzyl-4-carbamoyl-1,2,3,4-tetrahydrocarbaole-5-yl]oxyacetic acid;
{9-[(2-Pyridyl)methyl]-5-carbamoylcarbazol-4-yl}oxyacetic acid;
{9-[(3-Pyridyl)methyl]-5-carbamoylcarbazol-4-yl}oxyacetic acid;
[9-benzyl-4-carbamoyl-8-methyl-1,2,3,4-tetrahydrocarbazol-5-yl]oxyacetic acid;
[9-benzyl-5-carbamoyl-1-methylcarbazol-4-yl]oxyacetic acid;
[9-benzyl-4-carbamoyl-8-fluoro-1,2,3,4-tetrahydrocarbazol-5-yl]oxyacetic acid;
[9-benzyl-5-carbamoyl-1-fluorocarbazol-4-yl]oxyacetic acid;
[9-benzyl-4-carbamoyl-8-chloro-1,2,3,4-tetrahydrocarbazol-5-yl]oxyacetic acid;
[9-benzyl-5-carbamoyl-1-chlorocarbazol-4-yl]oxyacetic acid;
[9-[(Cyclohexyl)methyl]-5-carbamoylcarbazol-4-yl]oxyacetic acid;
[9-[(Cyclopentyl)methyl]-5-carbamoylcarbazol-4-yl]oxyacetic acid;
5-carbamoyl-9-(phenylmethyl)-2-[[(propen-3-yl)oxy]methyl]carbazol-4-yl]oxyacetic acid;
[5-carbamoyl-9-(phenylmethyl)-2-[(propyloxy)methyl]carbazol-4-yl]oxyacetic acid;
9-benzyl-7-methoxy-5-((carboxamidomethyl)oxy)-1,2,3,4-tetrahydrocarbazole-4-carboxamide;
9-benzyl-7-methoxy-5-cyanomethyloxy-carbazole-4-carboxamide;
9-benzyl-7-methoxy-5-((1H-tetrazol-5-yl-methyl)oxy)carbazole-4-carboxamide;
9-benzyl-7-methoxy-5-((carboxamidomethyl)oxy)-carbazole-4-carboxamide; and
[9-Benzyl-4-carbamoyl-1,2,3,4-tetrahydrocarbaole-5-yl]oxyacetic acid
or a pharmaceutically acceptable racemate, solvate, tautomer, optical isomer, prodrug derivative, or salt thereof.

38. Use of a compoud of formula I or II according to the method of claim 1, 2, 3, 4, 5, 6, or 7, for the manufacture of a medicament for the treatment of sepsis wherein the compound of formula I or II is selected from the group consisting of: 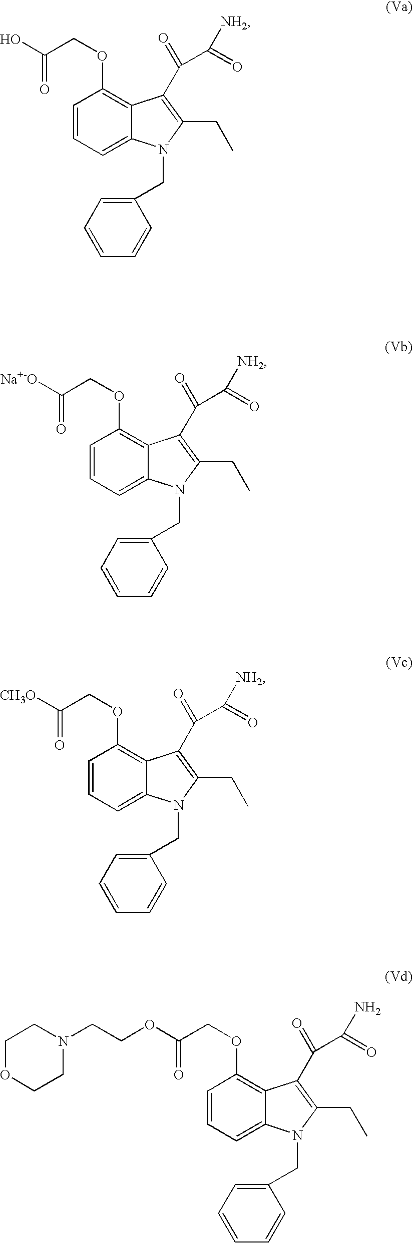 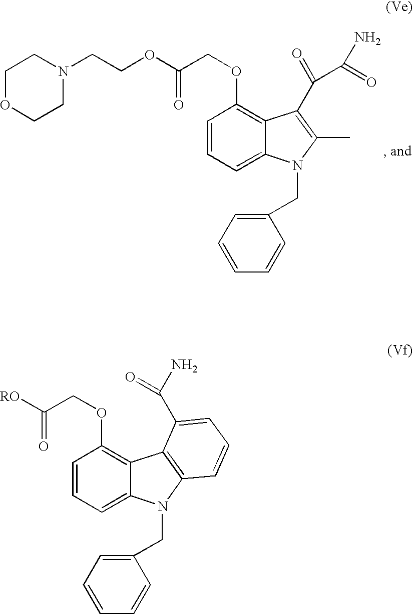
wherein R is methyl, ethyl, sodium ion, or N-morpholinoethyl group.

39. Use of a compound of formula (I) or (II) according to any of claims 1 to 7 for the manufacture of a medicament for the treatment of sepsis wherein the compound is [chemical structure]
